(19) United States
(12) Patent Application Publication
(10) Pub. No.: US 2024/0173300 A1
(43) Pub. Date: May 30, 2024

(54) ALPHA-2-ADRENERGIC RECEPTOR AGONISTS FOR REDUCING REDNESS AND INCREASING WHITENESS IN EYES AND OTHER OPHTHALMIC PURPOSES

(71) Applicant: Allergan, Inc., North Chicago, IL (US)

(72) Inventor: Mohammed Dibas, Corona, CA (US)

(21) Appl. No.: 18/335,032

(22) Filed: Jun. 14, 2023

Related U.S. Application Data
(63) Continuation of application No. 17/180,545, filed on Feb. 19, 2021, now abandoned.
(60) Provisional application No. 62/979,218, filed on Feb. 20, 2020.

(51) Int. Cl. A61K 31/4184; A61K 9/00; A61K 9/08; A61P 27/02

(52) U.S. Cl. CPC A61K 31/4184; A61K 9/0048; A61K 9/08; A61P 27/02

(57) ABSTRACT

The disclosure provides, inter alia, methods of reducing eye redness and/or increasing eye whiteness using alpha-2-adrenergic receptor agonists.

### CROSS-REFERENCE TO RELATED APPLICATIONS

This application is a continuation of U.S. patent application Ser. No. 17/180,545, filed Feb. 19, 2021, which claims the benefit of U.S. Provisional Application 62/979,218 filed Feb. 20, 2020, which are herein incorporated by reference in their entireties and serve as the basis of a priority and/or benefit claim for the present application.

### FIELD

The present disclosure relates to compound, pharmaceutical compositions, and methods for ocular vasoconstriction, reducing ocular hyperemia, reducing eye redness, increasing eye whiteness in individuals and/or other ophthalmic purposes. More particularly, disclosure relates generally to alpha-2-adrenergic receptor agonists, pharmaceutical compositions of alpha-2-adrenergic receptor agonists, and their use for ocular vasoconstriction, reducing ocular hyperemia, reducing eye redness, increasing eye whiteness and/or other ophthalmic purposes in individuals.

### BACKGROUND

Red eye is a common condition arising from surface vessel dilation (hyperemia) in the eye, particularly of the conjunctiva, tenon, and episclera/sclera layers. The ocular inflammation underlying red eye can be caused by several conditions including dry eyes, blepharitis, infection, inflammation (such as uveitis), injury (surgical or non-surgical, such as corneal abrasion), contact lens wear, allergy, corneal ulcer, and sub-conjunctival hemorrhage. Treatment of red eye is thus sometimes based on treatment of these causes—for example artificial tears for dry eyes, and antihistamine drops for allergic eyes.

Although some of the causes of red eye are relatively benign, ocular redness is generally a condition associated with reduced quality of life and negative social connotations such as drinking and drug abuse, in addition to general fatigue and cosmetic concerns. The most common treatment or relief of red eye itself is by the use of vasoconstrictors, which shrink the small blood vessels in the conjunctiva. Over-the-counter vasoconstrictors have been approved for relief of eye redness due to minor eye irritations. Vasoconstriction provides temporary relief from tissue congestion.

Over-the-counter products for treating eye redness may be associated with unwanted pharmacological and clinical phenomena, such as tachyphylaxis, tolerance, rebound vasodilation, toxicity, and abuse. There is a need in the art for compounds and pharmaceutical compositions that overcome the problems associated with currently available commercial products. The disclosure is directed to these, as well as other, important ends.

### SUMMARY

Disclosed herein are compounds (e.g. a compound of Formula I), pharmaceutical compositions, and methods for ocular vasoconstriction, reducing ocular hyperemia, reducing eye redness, and/or increasing eye whiteness with alpha-2-adrenergic receptor agonists.

In particular, described herein are compounds, pharmaceutical compositions, and methods for ocular vasoconstriction, reducing ocular hyperemia, reducing eye redness, and/or increasing eye whiteness by administering to an individual a therapeutically effective amount of a compound of Formula I:

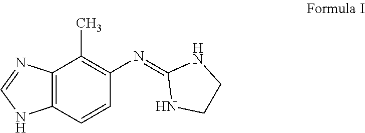 [SMILES: Cc1c(N=C2NCCN2)ccc2[nH]cnc12]

or a pharmaceutically acceptable salt thereof.

In addition, described herein are methods for ocular vasoconstriction, reducing ocular hyperemia, reducing eye redness and/or increasing eye whiteness by administering to an individual a therapeutically effective amount of a composition comprising a compound of Formula I:

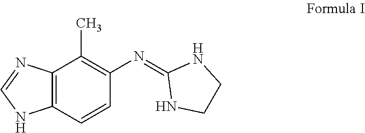 [SMILES: Cc1c(N=C2NCCN2)ccc2[nH]cnc12]

or a pharmaceutically acceptable salt thereof, and a pharmaceutically acceptable excipient.

Some non-limiting example embodiments are given below.

Example embodiment 1: A method for reducing redness of an eye or increasing whiteness of an eye in an individual in need thereof, the method comprising administering to the individual a therapeutically effective amount of a compound of Formula I:

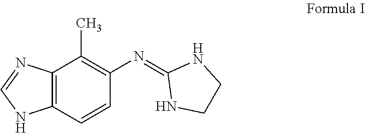 [SMILES: Cc1c(N=C2NCCN2)ccc2[nH]cnc12]

or a pharmaceutically acceptable salt thereof.

Example embodiment 2: The method of example embodiment 1, wherein the method is for reducing redness of the eye.

Example embodiment 3: The method of example embodiment 2, wherein the method is for reducing redness of the eye caused by minor eye irritations.

Example embodiment 4: The method of example embodiment 2, wherein the method is for reducing redness of the eye due to dilation of vessels of the conjunctiva, the episclera, the sclera, or a combination of two or more thereof.

Example embodiment 5: The method of any one of example embodiments 2 to 4, wherein the method is for reducing chronic redness of the eye.

Example embodiment 6: The method of example embodiment 1, wherein the method is for increasing whiteness of the eye.

Example embodiment 7: The method of example embodiment 6, wherein the individual does not have an eye pathology.

Example embodiment 8: The method of any one of example embodiments 1 to 7, wherein the method is for reducing redness of the eye caused by laser-assisted in situ keratomileusis eye surgery.

Example embodiment 9: The method of any one of example embodiments 1 to 8, wherein the method reduces redness of the eye or increases whiteness of the eye within 5 seconds of administration.

Example embodiment 10: The method of any one of example embodiments 1 to 8, wherein the method reduces redness of the eye or increases whiteness of the eye within 15 seconds of administration.

Example embodiment 11: The method of any one of example embodiments 1 to 8, wherein the method reduces redness of the eye or increases whiteness of the eye within 30 seconds of administration.

Example embodiment 12: The method of any one of example embodiments 1 to 8, wherein the method reduces redness of the eye or increases whiteness of the eye within 1 minute of administration.

Example embodiment 13: The method of any one of example embodiments 1 to 12, wherein the method reduces redness of the eye or increases whiteness of the eye for at least 8 hours after administration.

Example embodiment 14: The method of any one of example embodiments 1 to 12, wherein the method reduces redness of the eye or increases whiteness of the eye for at least 9 hours after administration.

Example embodiment 15: The method of any one of example embodiments 1 to 12, wherein the method reduces redness of the eye or increases whiteness of the eye for at least 10 hours after administration.

Example embodiment 16: The method of any one of example embodiments 1 to 12, wherein the method reduces redness of the eye or increases whiteness of the eye for at least 12 hours after administration.

Example embodiment 17: The method of any one of example embodiments 1 to 12, wherein the method reduces redness of the eye or increases whiteness of the eye for at least 24 hours after administration.

Example embodiment 18: The method of any one of example embodiments 1 to 17, wherein the compound of Formula I or the pharmaceutically acceptable salt thereof is administered to one eye of the individual or to both eyes of the individual.

Example embodiment 19: The method of any one of example embodiments 1 to 18, comprising topically administering to the eye of the individual the therapeutically effective amount of the compound of Formula I or the pharmaceutically acceptable salt thereof.

Example embodiment 20: The method of any one of example embodiments 1 to 19, wherein the compound of Formula I or the pharmaceutically acceptable salt thereof is administered to the individual as a pharmaceutical composition comprising the therapeutically effective amount of the compound of Formula I or the pharmaceutically acceptable salt thereof and a pharmaceutically acceptable excipient.

Example embodiment 21: The method of example embodiment 20, wherein the pharmaceutical composition comprises the compound of Formula I or the pharmaceutically acceptable salt thereof in an amount of about 0.001% (w/v) to about 0.02% (w/v).

Example embodiment 22: The method of example embodiment 20, wherein the pharmaceutical composition comprises the compound of Formula I or the pharmaceutically acceptable salt thereof in an amount of about 0.005% (w/v) to about 0.5% (w/v).

Example embodiment 23: The method of example embodiment 20, wherein the pharmaceutical composition comprises the compound of Formula I or the pharmaceutically acceptable salt thereof in an amount of about 0.01% (w/v).

Example embodiment 24: The method of example embodiment 20, wherein the pharmaceutical composition comprises the compound of Formula I or the pharmaceutically acceptable salt thereof in an amount of about 0.02% (w/v).

Example embodiment 25: The method of example embodiment 20, wherein the pharmaceutical composition comprises the compound of Formula I or the pharmaceutically acceptable salt thereof in an amount of about 0.03% (w/v).

Example embodiment 26: The method of example embodiment 20, wherein the pharmaceutical composition comprises the compound of Formula I or the pharmaceutically acceptable salt thereof in an amount of 0.1% (w/v).

Example embodiment 27: The method of example embodiment 20, wherein the pharmaceutical composition comprises the compound of Formula I or the pharmaceutically acceptable salt thereof in an amount of 0.3% (w/v).

Example embodiment 28: The method of any one of example embodiments 20 to 27, wherein the pharmaceutical composition is a solution.

Example embodiment 29: The method of any one of example embodiments 20 to 27, wherein the pharmaceutical composition is a dispersion.

Example embodiment 30: The method of any one of example embodiments 20 to 29, comprising topically administering to the eye of the individual one drop of the pharmaceutical composition.

Example embodiment 31: The method of any one of example embodiments 20 to 29, comprising topically administering to the eye of the individual two drops of the pharmaceutical composition.

Example embodiment 32: The method of example embodiment 20, wherein the pharmaceutical composition is an ocular implant, intracameral implant, intravitreal implant, sub-conjunctival implant, sub-Tenon's implant, punctum plug, canicular eluting implant, or ocular ring.

Example embodiment 33: The method of example embodiment 20, wherein the pharmaceutical composition comprises microspheres which comprise the compound of Formula (I) or the pharmaceutically acceptable salt thereof.

Example embodiment 34: The method of example embodiment 20, wherein the therapeutically effective amount of the compound of Formula (I) or the pharmaceutically acceptable salt thereof is an amount that is less than a therapeutically amount of brimonidine that is needed to reduce redness of the eye or to increase whiteness of the eye.

Example embodiment 35: A compound of Formula I:

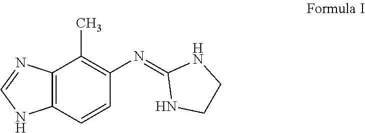 [SMILES: Cc1c(N=C2NCCN2)ccc2[nH]cnc12]

or a pharmaceutically acceptable salt thereof, for use in a method for reducing redness of an eye or increasing whiteness of an eye in an individual in need thereof, the method comprising administering to the individual a therapeutically effective amount of a compound of Formula I or a pharmaceutically acceptable salt thereof.

Example embodiment 36: The compound of pharmaceutically acceptable salt of example embodiment 35, wherein the method is for reducing redness of the eye.

Example embodiment 37: The compound of pharmaceutically acceptable salt of example embodiment 36, wherein the method is for reducing redness of the eye caused by minor eye irritations.

Example embodiment 38: The compound of pharmaceutically acceptable salt of example embodiment 36, wherein the method is for reducing redness of the eye due to dilation of vessels of the conjunctiva, the episclera, the sclera, or a combination of two or more thereof.

Example embodiment 39: The compound of pharmaceutically acceptable salt of any one of example embodiments 36 to 38, wherein the method is for reducing chronic redness of the eye.

Example embodiment 40: The compound of pharmaceutically acceptable salt of example embodiment 35, wherein the method is for increasing whiteness of the eye.

Example embodiment 41: The compound of pharmaceutically acceptable salt of example embodiment 40, wherein the individual does not have an eye pathology.

Example embodiment 42: The compound of pharmaceutically acceptable salt of any one of example embodiments 35 to 41, wherein the method is for reducing redness of the eye caused by laser-assisted in situ keratomileusis eye surgery.

Example embodiment 43: The compound of pharmaceutically acceptable salt of any one of example embodiments 35 to 42, wherein the method reduces redness of the eye or increases whiteness of the eye within 5 seconds of administration.

Example embodiment 44: The compound of pharmaceutically acceptable salt of any one of example embodiments 35 to 42, wherein the method reduces redness of the eye or increases whiteness of the eye within 15 seconds of administration.

Example embodiment 45: The compound of pharmaceutically acceptable salt of any one of example embodiments 35 to 42, wherein the method reduces redness of the eye or increases whiteness of the eye within 30 seconds of administration.

Example embodiment 46: The compound of pharmaceutically acceptable salt of any one of example embodiments 35 to 42, wherein the method reduces redness of the eye or increases whiteness of the eye within 1 minute of administration.

Example embodiment 47: The compound of pharmaceutically acceptable salt of any one of example embodiments 35 to 46, wherein the method reduces redness of the eye or increases whiteness of the eye for at least 8 hours after administration.

Example embodiment 48: The compound of pharmaceutically acceptable salt of any one of example embodiments 35 to 46, wherein the method reduces redness of the eye or increases whiteness of the eye for at least 9 hours after administration.

Example embodiment 49: The compound of pharmaceutically acceptable salt of any one of example embodiments 35 to 46, wherein the method reduces redness of the eye or increases whiteness of the eye for at least 10 hours after administration.

Example embodiment 50: The compound of pharmaceutically acceptable salt of any one of example embodiments 35 to 46, wherein the method reduces redness of the eye or increases whiteness of the eye for at least 12 hours after administration.

Example embodiment 51: The compound of pharmaceutically acceptable salt of any one of example embodiments 35 to 46, wherein the method reduces redness of the eye or increases whiteness of the eye for at least 24 hours after administration.

Example embodiment 52: The compound of pharmaceutically acceptable salt of any one of example embodiments 35 to 51, wherein the compound of Formula I or the pharmaceutically acceptable salt thereof is administered to one eye of the individual or to both eyes of the individual.

Example embodiment 53: The compound of pharmaceutically acceptable salt of any one of example embodiments 35 to 52, comprising topically administering to the eye of the individual the therapeutically effective amount of the compound of Formula I or the pharmaceutically acceptable salt thereof.

Example embodiment 54: The compound of pharmaceutically acceptable salt of any one of example embodiments 35 to 53, wherein the compound of Formula I or the pharmaceutically acceptable salt thereof is administered to the individual as a pharmaceutical composition comprising the therapeutically effective amount of the compound of Formula I or the pharmaceutically acceptable salt thereof and a pharmaceutically acceptable excipient.

Example embodiment 55: The compound of pharmaceutically acceptable salt of example embodiment 54, wherein the pharmaceutical composition comprises the compound of Formula I or the pharmaceutically acceptable salt thereof in an amount of about 0.001% (w/v) to about 0.02% (w/v).

Example embodiment 56: The compound of pharmaceutically acceptable salt of example embodiment 54, wherein the pharmaceutical composition comprises the compound of Formula I or the pharmaceutically acceptable salt thereof in an amount of about 0.005% (w/v) to about 0.5% (w/v).

Example embodiment 57: The compound of pharmaceutically acceptable salt of example embodiment 54, wherein the pharmaceutical composition comprises the compound of Formula I or the pharmaceutically acceptable salt thereof in an amount of about 0.01% (w/v).

Example embodiment 58: The compound of pharmaceutically acceptable salt of example embodiment 54, wherein the pharmaceutical composition comprises the compound of Formula I or the pharmaceutically acceptable salt thereof in an amount of about 0.02% (w/v).

Example embodiment 59: The compound of pharmaceutically acceptable salt of example embodiment 54, wherein the pharmaceutical composition comprises the compound of Formula I or the pharmaceutically acceptable salt thereof in an amount of about 0.03% (w/v).

Example embodiment 60: The compound of pharmaceutically acceptable salt of example embodiment 54, wherein the pharmaceutical composition comprises the compound of Formula I or the pharmaceutically acceptable salt thereof in an amount of 0.1% (w/v).

Example embodiment 61: The compound of pharmaceutically acceptable salt of example embodiment 54, wherein the pharmaceutical composition comprises the compound of Formula I or the pharmaceutically acceptable salt thereof in an amount of 0.3% (w/v).

Example embodiment 62: The compound of pharmaceutically acceptable salt of any one of example embodiments 54 to 61, wherein the pharmaceutical composition is a solution.

Example embodiment 63: The compound of pharmaceutically acceptable salt of any one of example embodiments 54 to 61, wherein the pharmaceutical composition is a dispersion.

Example embodiment 64: The compound of pharmaceutically acceptable salt of any one of example embodiments 54 to 63, comprising topically administering to the eye of the individual one drop of the pharmaceutical composition.

Example embodiment 65: The compound of pharmaceutically acceptable salt of any one of example embodiments 54 to 63, comprising topically administering to the eye of the individual two drops of the pharmaceutical composition.

Example embodiment 66: The compound of pharmaceutically acceptable salt of example embodiment 54, wherein the pharmaceutical composition is an ocular implant, intracameral implant, intravitreal implant, sub-conjunctival implant, sub-Tenon's implant, punctum plug, canicular eluting implant, or ocular ring.

Example embodiment 67: The compound of pharmaceutically acceptable salt of example embodiment 54, wherein the pharmaceutical composition comprises microspheres which comprise the compound of Formula (I) or the pharmaceutically acceptable salt thereof.

Example embodiment 68: The compound of pharmaceutically acceptable salt of example embodiment 54, wherein the therapeutically effective amount of the compound of Formula (I) or the pharmaceutically acceptable salt thereof is an amount that is less than a therapeutically amount of brimonidine that is needed to reduce redness of the eye or to increase whiteness of the eye.

Example embodiment 69: Use of a compound of Formula I:

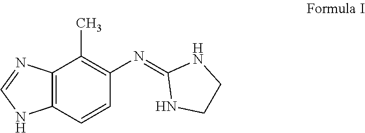 [SMILES: Cc1c(N=C2NCCN2)ccc2[nH]cnc12]

or a pharmaceutically acceptable salt thereof, in a method for reducing redness of an eye or increasing whiteness of an eye in an individual in need thereof, the method comprising administering to the individual a therapeutically effective amount of a compound of Formula I or a pharmaceutically acceptable salt thereof.

Example embodiment 70: The use of example embodiment 69, wherein the method is for reducing redness of the eye.

Example embodiment 71: The use of example embodiment 70, wherein the method is for reducing redness of the eye caused by minor eye irritations.

Example embodiment 72: The use of example embodiment 70, wherein the method is for reducing redness of the eye due to dilation of vessels of the conjunctiva, the episclera, the sclera, or a combination of two or more thereof.

Example embodiment 73: The use of any one of example embodiments 70 to 72, wherein the method is for reducing chronic redness of the eye.

Example embodiment 74: The use of example embodiment 69, wherein the method is for increasing whiteness of the eye.

Example embodiment 75: The use of example embodiment 74, wherein the individual does not have an eye pathology.

Example embodiment 76: The use of any one of example embodiments 69 to 74, wherein the method is for reducing redness of the eye caused by laser-assisted in situ keratomileusis eye surgery.

Example embodiment 77: The use of any one of example embodiments 69 to 76, wherein the method reduces redness of the eye or increases whiteness of the eye within 5 seconds of administration.

Example embodiment 78: The use of any one of example embodiments 69 to 76, wherein the method reduces redness of the eye or increases whiteness of the eye within 15 seconds of administration.

Example embodiment 79: The use of any one of example embodiments 69 to 76, wherein the method reduces redness of the eye or increases whiteness of the eye within 30 seconds of administration.

Example embodiment 80: The use of any one of example embodiments 69 to 76, wherein the method reduces redness of the eye or increases whiteness of the eye within 1 minute of administration.

Example embodiment 81: The use of any one of example embodiments 69 to 80, wherein the method reduces redness of the eye or increases whiteness of the eye for at least 8 hours after administration.

Example embodiment 82: The use of any one of example embodiments 69 to 80, wherein the method reduces redness of the eye or increases whiteness of the eye for at least 9 hours after administration.

Example embodiment 83: The use of any one of example embodiments 69 to 80, wherein the method reduces redness of the eye or increases whiteness of the eye for at least 10 hours after administration.

Example embodiment 84: The use of any one of example embodiments 69 to 80, wherein the method reduces redness of the eye or increases whiteness of the eye for at least 12 hours after administration.

Example embodiment 85: The use of any one of example embodiments 69 to 80, wherein the method reduces redness of the eye or increases whiteness of the eye for at least 24 hours after administration.

Example embodiment 86: The use of any one of example embodiments 69 to 85, wherein the compound of Formula I or the pharmaceutically acceptable salt thereof is administered to one eye of the individual or to both eyes of the individual.

Example embodiment 87: The use of any one of example embodiments 69 to 86, comprising topically administering to the eye of the individual the therapeutically effective amount of the compound of Formula I or the pharmaceutically acceptable salt thereof.

Example embodiment 88: The use of any one of example embodiments 69 to 87, wherein the compound of Formula I or the pharmaceutically acceptable salt thereof is administered to the individual as a pharmaceutical composition comprising the therapeutically effective amount of the compound of Formula I or the pharmaceutically acceptable salt thereof and a pharmaceutically acceptable excipient.

Example embodiment 89: The use of example embodiment 88, wherein the pharmaceutical composition comprises the compound of Formula I or the pharmaceutically acceptable salt thereof in an amount of about 0.001% (w/v) to about 0.02% (w/v).

Example embodiment 90: The use of example embodiment 88, wherein the pharmaceutical composition comprises the compound of Formula I or the pharmaceutically acceptable salt thereof in an amount of about 0.005% (w/v) to about 0.5% (w/v).

Example embodiment 91: The use of example embodiment 88, wherein the pharmaceutical composition comprises the compound of Formula I or the pharmaceutically acceptable salt thereof in an amount of about 0.01% (w/v).

Example embodiment 92: The use of example embodiment 88, wherein the pharmaceutical composition comprises the compound of Formula I or the pharmaceutically acceptable salt thereof in an amount of about 0.02% (w/v).

Example embodiment 93: The use of example embodiment 88, wherein the pharmaceutical composition comprises the compound of Formula I or the pharmaceutically acceptable salt thereof in an amount of about 0.03% (w/v).

Example embodiment 94: The use of example embodiment 88, wherein the pharmaceutical composition comprises the compound of Formula I or the pharmaceutically acceptable salt thereof in an amount of 0.1% (w/v).

Example embodiment 95: The use of example embodiment 88, wherein the pharmaceutical composition comprises the compound of Formula I or the pharmaceutically acceptable salt thereof in an amount of 0.3% (w/v).

Example embodiment 96: The use of any one of example embodiments 88 to 95, wherein the pharmaceutical composition is a solution.

Example embodiment 97: The use of any one of example embodiments 88 to 95, wherein the pharmaceutical composition is a dispersion.

Example embodiment 98: The use of any one of example embodiments 88 to 97, comprising topically administering to the eye of the individual one drop of the pharmaceutical composition.

Example embodiment 99: The use of any one of example embodiments 88 to 97, comprising topically administering to the eye of the individual two drops of the pharmaceutical composition.

Example embodiment 100: The use of example embodiment 88, wherein the pharmaceutical composition is an ocular implant, intracameral implant, intravitreal implant, sub-conjunctival implant, sub-Tenon's implant, punctum plug, canicular eluting implant, or ocular ring.

Example embodiment 101: The use of example embodiment 88, wherein the pharmaceutical composition comprises microspheres which comprise the compound of Formula (I) or the pharmaceutically acceptable salt thereof.

Example embodiment 102: The use of example embodiment 88, wherein the therapeutically effective amount of the compound of Formula (I) or the pharmaceutically acceptable salt thereof is an amount that is less than a therapeutically amount of brimonidine that is needed to reduce redness of the eye or to increase whiteness of the eye.

Example embodiment 103: Use of a compound of Formula I:

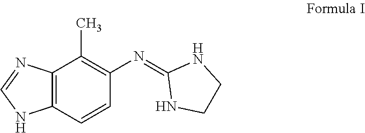 [SMILES: Cc1c(N=C2NCCN2)ccc2[nH]cnc12]

or a pharmaceutically acceptable salt thereof, in the manufacture of a medicament for reducing redness of an eye or increasing whiteness of an eye in an individual in need thereof, wherein the medicament comprises a therapeutically effective amount of a compound of Formula I or a pharmaceutically acceptable salt thereof.

Example embodiment 104: The use of example embodiment 103, wherein the medicament is for reducing redness of the eye.

Example embodiment 105: The use of example embodiment 104, wherein the medicament is for reducing redness of the eye caused by minor eye irritations.

Example embodiment 106: The use of example embodiment 104, wherein the medicament is for reducing redness of the eye due to dilation of vessels of the conjunctiva, the episclera, the sclera, or a combination of two or more thereof.

Example embodiment 107: The use of any one of example embodiments 104 to 106, wherein the medicament is for reducing chronic redness of the eye.

Example embodiment 108: The use of example embodiment 103, wherein the medicament is for increasing whiteness of the eye.

Example embodiment 109: The use of example embodiment 108, wherein the individual does not have an eye pathology.

Example embodiment 110: The use of any one of example embodiments 103 to 108, wherein the medicament is for reducing redness of the eye caused by laser-assisted in situ keratomileusis eye surgery.

Example embodiment 111: The use of any one of example embodiments 103 to 110, wherein the medicament reduces redness of the eye or increases whiteness of the eye within 5 seconds of administration.

Example embodiment 112: The use of any one of example embodiments 103 to 110, wherein the medicament reduces redness of the eye or increases whiteness of the eye within 15 seconds of administration.

Example embodiment 113: The use of any one of example embodiments 103 to 110, wherein the medicament reduces redness of the eye or increases whiteness of the eye within 30 seconds of administration.

Example embodiment 114: The use of any one of example embodiments 103 to 110, wherein the medicament reduces redness of the eye or increases whiteness of the eye within 1 minute of administration.

Example embodiment 115: The use of any one of example embodiments 103 to 114, wherein the medicament reduces redness of the eye or increases whiteness of the eye for at least 8 hours after administration.

Example embodiment 116: The use of any one of example embodiments 103 to 114, wherein the medicament reduces redness of the eye or increases whiteness of the eye for at least 9 hours after administration.

Example embodiment 117: The use of any one of example embodiments 103 to 114, wherein the medicament reduces redness of the eye or increases whiteness of the eye for at least 10 hours after administration.

Example embodiment 118: The use of any one of example embodiments 103 to 114, wherein the medicament reduces redness of the eye or increases whiteness of the eye for at least 12 hours after administration.

Example embodiment 119: The use of any one of example embodiments 103 to 114, wherein the medicament reduces redness of the eye or increases whiteness of the eye for at least 24 hours after administration.

Example embodiment 120: The use of any one of example embodiments 103 to 119, wherein the medicament, when administered to an individual in need thereof, is administered to one eye of the individual or to both eyes of the individual.

Example embodiment 121: The use of any one of example embodiments 103 to 120, wherein the medicament, when administered to an individual in need thereof, is administered topically to the eye of an individual in need thereof.

Example embodiment 122: The use of any one of example embodiments 103 to 121, wherein the medicament is a pharmaceutical composition comprising the therapeutically effective amount of the compound of Formula I or the pharmaceutically acceptable salt thereof and a pharmaceutically acceptable excipient.

Example embodiment 123: The use of example embodiment 122, wherein the pharmaceutical composition comprises the compound of Formula I or the pharmaceutically acceptable salt thereof in an amount of about 0.001% (w/v) to about 0.02% (w/v).

Example embodiment 124: The use of example embodiment 122, wherein the pharmaceutical composition comprises the compound of Formula I or the pharmaceutically acceptable salt thereof in an amount of about 0.005% (w/v) to about 0.5% (w/v).

Example embodiment 125: The use of example embodiment 122, wherein the pharmaceutical composition comprises the compound of Formula I or the pharmaceutically acceptable salt thereof in an amount of about 0.01% (w/v).

Example embodiment 126: The use of example embodiment 122, wherein the pharmaceutical composition comprises the compound of Formula I or the pharmaceutically acceptable salt thereof in an amount of about 0.02% (w/v).

Example embodiment 127: The use of example embodiment 122, wherein the pharmaceutical composition comprises the compound of Formula I or the pharmaceutically acceptable salt thereof in an amount of about 0.03% (w/v).

Example embodiment 128: The use of example embodiment 122, wherein the pharmaceutical composition comprises the compound of Formula I or the pharmaceutically acceptable salt thereof in an amount of 0.1% (w/v).

Example embodiment 129: The use of example embodiment 122, wherein the pharmaceutical composition comprises the compound of Formula I or the pharmaceutically acceptable salt thereof in an amount of 0.3% (w/v).

Example embodiment 130: The use of any one of example embodiments 122 to 129, wherein the pharmaceutical composition is a solution.

Example embodiment 131: The use of any one of example embodiments 122 to 129, wherein the pharmaceutical composition is a dispersion.

Example embodiment 132: The use of any one of example embodiments 122 to 131, wherein the pharmaceutical composition, when administered to an individual in need thereof, is administered as one drop topically to the individual.

Example embodiment 133: The use of any one of example embodiments 122 to 131, wherein the pharmaceutical composition, when administered to an individual in need thereof, is administered as two drops topically to the individual.

Example embodiment 134: The use of example embodiment 122, wherein the pharmaceutical composition is an ocular implant, intracameral implant, intravitreal implant, sub-conjunctival implant, sub-Tenon's implant, punctum plug, canicular eluting implant, or ocular ring.

Example embodiment 135: The use of example embodiment 122, wherein the pharmaceutical composition comprises microspheres which comprise the compound of Formula (I) or the pharmaceutically acceptable salt thereof.

Example embodiment 136: The use of example embodiment 122, wherein the therapeutically effective amount of the compound of Formula (I) or the pharmaceutically acceptable salt thereof is an amount that is less than a therapeutically amount of brimonidine that is needed to reduce redness of the eye or to increase whiteness of the eye.

Example embodiment 137: A method for reducing redness of an eye or increasing whiteness of an eye substantially as described herein.

Example embodiment 138: A method for reducing redness of an eye or increasing whiteness of an eye with a compound of Formula I:

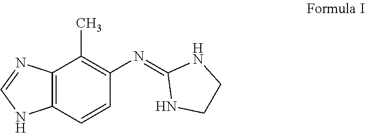 [SMILES: Cc1c(N=C2NCCN2)ccc2[nH]cnc12]

or a pharmaceutically acceptable salt thereof substantially as described herein.

Example embodiment 139: A method of using a compound of Formula I:

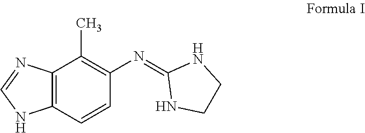 [SMILES: Cc1c(N=C2NCCN2)ccc2[nH]cnc12]

or a pharmaceutically acceptable salt thereof substantially as described herein.

Some non-limiting additional example embodiments are given below.

Additional example embodiment 1: A pharmaceutical composition comprising a compound of Formula I:

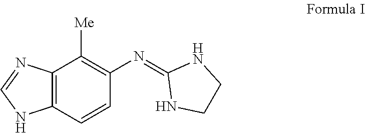 [SMILES: Cc1c(N=C2NCCN2)ccc2[nH]cnc12]

or a pharmaceutically acceptable salt thereof, a buffer, sodium chloride, and water.

Additional example embodiment 2: The pharmaceutical composition of additional example embodiment 1, wherein the compound of Formula I is present in the composition in an amount of between about 0.003% and about 1% (w/v).

Additional example embodiment 3: The pharmaceutical composition of additional example embodiment 2, wherein the compound of Formula I is present in the composition in an amount of about 0.01% (w/v).

Additional example embodiment 4: The pharmaceutical composition of additional example embodiment 2, wherein the compound of Formula I is present in the composition in an amount of about 0.03% (w/v).

Additional example embodiment 5: The pharmaceutical composition of additional example embodiment 2, wherein the compound of Formula I is present in the composition in an amount of about 0.1% (w/v).

Additional example embodiment 6: The pharmaceutical composition of additional example embodiment 2, wherein the compound of Formula I is present in the composition in an amount of about 0.3% (w/v).

Additional example embodiment 7: The pharmaceutical composition of any one of additional example embodiments 1 to 6, wherein the buffer is a citrate buffer and phosphate buffer.

Additional example embodiment 8: The pharmaceutical composition of additional example embodiment 7, wherein the citrate buffer comprises citric acid and the phosphate buffer comprises dibasic sodium phosphate.

Additional example embodiment 9: The pharmaceutical composition of any one of additional example embodiment 8, wherein the citric acid is present in the composition in an amount of between about 0.01% and 1% (w/v).

Additional example embodiment 10: The pharmaceutical composition of any one of additional example embodiments 8 or 9, wherein the citric acid is present in the composition in an amount of about 0.1% (w/v).

Additional example embodiment 11: The pharmaceutical composition of any one of additional example embodiments 8 or 9, wherein the citric acid is present in the composition in an amount of about 0.09% (w/v).

Additional example embodiment 12: The pharmaceutical composition of any one of additional example embodiments 8 to 11, wherein the dibasic sodium phosphate is present in the composition in an amount of between about 0.01% and 2% (w/v).

Additional example embodiment 13: The pharmaceutical composition of any one of additional example embodiments 8 to 12, wherein the dibasic sodium phosphate is present in the composition in an amount of about 0.5% (w/v).

Additional example embodiment 14: The pharmaceutical composition of any one of additional example embodiments 8 to 12, wherein the dibasic sodium phosphate is present in the composition in an amount of about 1% (w/v).

Additional example embodiment 15: The pharmaceutical composition of any one of additional example embodiments 1 to 14, wherein the sodium chloride is present in the composition in amount of at least 0.6% (w/v).

Additional example embodiment 16: The pharmaceutical composition of any one of additional example embodiments 1 to 14, wherein the sodium chloride is present in the composition in amount of about 0.6% (w/v).

Additional example embodiment 17: The pharmaceutical composition of any one of additional example embodiments 1 to 16, wherein the composition has a pH of between about 4.5 and about 8.

Additional example embodiment 18: The pharmaceutical composition of any one of additional example embodiments 1 to 17, wherein the composition has a pH of between about 6.5 and about 7.6.

Additional example embodiment 19: The pharmaceutical composition of any one of additional example embodiments 1 to 18, wherein the composition has a pH of about 7.

Additional example embodiment 20: The pharmaceutical composition of additional example embodiment 1, wherein the compound of Formula I is present in the composition in an amount of about 0.01% (w/v), the sodium chloride is present in the composition in an amount of at least 0.6%, the buffer is a citrate buffer comprising citric acid and a phosphate buffer comprising dibasic sodium phosphate.

Additional example embodiment 21: The pharmaceutical composition of additional example embodiment 16, wherein the citric acid is present in the composition in amount of about 0.1% (w/v) and the dibasic sodium phosphate is present in the composition in an amount of 1% (w/v).

Additional example embodiment 22: The pharmaceutical composition of additional example embodiment 16, wherein the citric acid is present in the composition in amount of about 0.09% (w/v) and the dibasic sodium phosphate is present in the composition in an amount of 0.5% (w/v).

Additional example embodiment 23: The pharmaceutical composition of additional example embodiment 1, wherein the compound of Formula I is present in the composition in an amount of about 0.03% (w/v), the sodium chloride is present in the composition in an amount of at least 0.6%, the buffer is a citrate buffer comprising citric acid and a phosphate buffer comprising dibasic sodium phosphate.

Additional example embodiment 24: The pharmaceutical composition of additional example embodiment 16, wherein the citric acid is present in the composition in amount of about 0.1% (w/v) and the dibasic sodium phosphate is present in the composition in an amount of 1% (w/v).

Additional example embodiment 25: The pharmaceutical composition of additional example embodiment 16, wherein the citric acid is present in the composition in amount of about 0.09% (w/v) and the dibasic sodium phosphate is present in the composition in an amount of 0.5% (w/v).

Additional example embodiment 26: The pharmaceutical composition of additional example embodiment 1, wherein the compound of Formula I is present in the composition in an amount of about 0.1% (w/v), the sodium chloride is present in the composition in an amount of at least 0.6%, the buffer is a citrate buffer comprising citric acid and a phosphate buffer comprising dibasic sodium phosphate.

Additional example embodiment 27: The pharmaceutical composition of additional example embodiment 16, wherein the citric acid is present in the composition in amount of about 0.1% (w/v) and the dibasic sodium phosphate is present in the composition in an amount of 1% (w/v).

Additional example embodiment 28: The pharmaceutical composition of additional example embodiment 16, wherein the citric acid is present in the composition in amount of about 0.09% (w/v) and the dibasic sodium phosphate is present in the composition in an amount of 0.5% (w/v).

Additional example embodiment 29: The pharmaceutical composition of additional example embodiment 1, wherein the compound of Formula I is present in the composition in an amount of about 0.3% (w/v), the sodium chloride is present in the composition in an amount of at least 0.6%, the buffer is a citrate buffer comprising citric acid and a phosphate buffer comprising dibasic sodium phosphate.

Additional example embodiment 30: The pharmaceutical composition of additional example embodiment 16, wherein the citric acid is present in the composition in amount of about 0.1% (w/v) and the dibasic sodium phosphate is present in the composition in an amount of 1% (w/v).

Additional example embodiment 31: The pharmaceutical composition of additional example embodiment 16, wherein the citric acid is present in the composition in amount of about 0.09% (w/v) and the dibasic sodium phosphate is present in the composition in an amount of 0.5% (w/v).

Additional example embodiment 32: A method of treating an ocular condition in an individual in need of such treatment, the method comprising administering to the individual the pharmaceutical composition of any one of additional example embodiments 1 to 31, and wherein the ocular condition is selected from the group consisting of presbyopia, poor night vision, visual glare, visual starbursts, visual halos, and night myopia.

Additional example embodiment 33: The method of additional example embodiment 32, wherein the ocular condition is presbyopia.

Additional example embodiment 34: The method of additional example embodiment 32, wherein the ocular condition is poor night vision.

Additional example embodiment 35: The method of additional example embodiment 32, wherein the ocular condition is visual glare.

Additional example embodiment 36: The method of additional example embodiment 33, wherein the ocular condition is visual starbursts.

Additional example embodiment 37: The method of additional example embodiment 33, wherein the ocular condition is visual halos.

Additional example embodiment 38: The method of additional example embodiment 33, wherein the ocular condition is night myopia.

Additional example embodiment 39: The method of any one of additional example embodiments 32 to 38, wherein the pharmaceutical composition is administered to one or both eyes of the individual.

Additional example embodiment 40: The method of additional example embodiment 39, wherein the administration to the eye is topical administration.

Additional example embodiment 41: The method of any one of additional example embodiment 33 to 40, wherein the compound of Formula I or pharmaceutically acceptable salt thereof, when administered to the individual, has binding to the iris pigment that is less than the binding to the iris pigment exhibited by brimonidine.

Additional example embodiment 42: The method of any one of additional example embodiments 33 to 40, wherein the amount of the compound of Formula I or pharmaceutically acceptable salt thereof is an amount that is less than the amount of brimonidine needed to achieve the same therapeutic effects.

Additional example embodiment 43: The method of any one of additional example embodiments 33 to 40, wherein the pharmaceutical composition, when administered to the individual, causes an amount of reduction in pupil size such that the pupil is constricted to a size of between 2 and 3 mm.

Additional example embodiment 44: The method of any one of additional example embodiments 33 to 40, wherein the pharmaceutical composition, when administered to the individual, causes an amount of reduction in pupil size such that the pupil is constricted to a size of 3 mm or less.

Additional example embodiment 45: The method of any one of additional example embodiments 33 to 40, wherein the pharmaceutical composition, when administered to the individual, causes an amount of reduction in pupil size such that the pupil is constricted to a size of 2.5 mm or less.

Additional example embodiment 46: The method of any one of additional example embodiments 33 to 40, wherein the pharmaceutical composition, when administered to the individual, causes an improvement in near visual acuity.

Additional example embodiment 47: The method of any one of additional example embodiments 33 to 40, wherein the pharmaceutical composition, when administered to the individual, causes an improvement in intermediate visual acuity.

Additional example embodiment 48: The method of any one of additional example embodiments 33 to 40, wherein the pharmaceutical composition, when administered to the individual, causes an improvement in distance visual acuity.

Additional example embodiment 49: The method of any one of additional example embodiments 46 to 48, where in the improvement in visual acuity is an at least 2-line improvement.

Additional example embodiment 50: The method of any one of additional example embodiments 46 to 48, where in the improvement in visual acuity is an at least 3-line improvement.

Additional example embodiment 51: The method of any one of additional example embodiments 43 to 50, wherein the reduction in pupil size or improvement in visual acuity is maintained for at least 1 hour.

Additional example embodiment 52: The method of any one of additional example embodiments 43 to 50, wherein the reduction in pupil size or improvement in visual acuity is maintained for at least 2 hours.

Additional example embodiment 53: The method of any one of additional example embodiments 43 to 50, wherein the reduction in pupil size or improvement in visual acuity is maintained for at least 4 hours.

Additional example embodiment 54: The method of any one of additional example embodiments 43 to 50, wherein the reduction in pupil size or improvement in visual acuity is maintained for at least 6 hours.

Additional example embodiment 55: The method of any one of additional example embodiments 43 to 50, wherein the reduction in pupil size or improvement in visual acuity is maintained for at least 9 hours.

Additional example embodiment 56: The method of any one of additional example embodiments 43 to 50, wherein the reduction in pupil size or improvement in visual acuity is maintained for at least 10 hours.

Additional example embodiment 57: The method of any one of additional example embodiments 43 to 50, wherein the reduction in pupil size or improvement in visual acuity is maintained for at least 12 hours.

Additional example embodiment 58: The method of any one of additional example embodiments 43 to 50, wherein the reduction in pupil size or improvement in visual acuity is achieved when the individual is exposed to luminance levels of less than 200 cd/m².

Additional example embodiment 59: The method of any one of additional example embodiments 43 to 50, wherein the reduction in pupil size or improvement in visual acuity is achieved when the individual is exposed to luminance levels of less than 150 cd/m².

Additional example embodiment 60: The method of any one of additional example embodiments 43 to 50, wherein the reduction in pupil size or improvement in visual acuity is achieved when the individual is exposed to luminance levels of less than 100 cd/m².

Additional example embodiment 61: The method of any one of additional example embodiments 43 to 60, wherein the reduction in pupil size or improvement in visual acuity is achieved when the individual is exposed to luminance levels of less than 50 cd/m².

Additional example embodiment 62: The method of any one of additional example embodiments 43 to 60, wherein the reduction in pupil size or improvement in visual acuity is achieved when the individual is exposed to luminance levels of less than 10 cd/m².

Additional example embodiment 63: The method of any one of additional example embodiments 43 to 60, wherein the reduction in pupil size or improvement in visual acuity is achieved when the individual is exposed to luminance levels of less than 5 cd/m².

Additional example embodiment 64: The method of any one of additional example embodiments 43 to 60, wherein the reduction in pupil size or improvement in visual acuity is achieved when the individual is exposed to luminance levels of less than 2 cd/m².

Additional example embodiment 65: The pharmaceutical composition of any one of additional example embodiments 1 to 31 for use in a method of treating an ocular condition in an individual in need thereof, the method comprising administering the pharmaceutical composition to the individual, and wherein the ocular condition is selected from the group consisting of presbyopia, poor night vision, visual glare, visual starbursts, visual halos, and night myopia.

Additional example embodiment 66: The pharmaceutical composition for use according to additional example embodiment 65, wherein the ocular condition is presbyopia.

Additional example embodiment 67: The pharmaceutical composition for use according to additional example embodiment 65, wherein the ocular condition is poor night vision.

Additional example embodiment 68: The pharmaceutical composition for use according to additional example embodiment 65, wherein the ocular condition is visual glare.

Additional example embodiment 69: The pharmaceutical composition for use according to additional example embodiment 65, wherein the ocular condition is visual starbursts.

Additional example embodiment 70: The pharmaceutical composition for use according to additional example embodiment 65, wherein the ocular condition is visual halos.

Additional example embodiment 71: The pharmaceutical composition for use according to additional example embodiment 65, wherein the ocular condition is night myopia.

Additional example embodiment 72: The pharmaceutical composition for use according to any one of additional example embodiments 65 to 71, wherein the pharmaceutical composition is administered to one or both eyes of the individual.

Additional example embodiment 73: The compound or pharmaceutically acceptable salt thereof for use according to additional example embodiment 72, wherein the administration to the eye is topical administration.

Additional example embodiment 74: The pharmaceutical composition for use according to any one of additional example embodiments 65 to 73, wherein the amount of the compound of Formula I or pharmaceutically acceptable salt thereof, when administered to the individual, has binding to the iris pigment that is less than the binding to the iris pigment exhibited by brimonidine.

Additional example embodiment 75: The pharmaceutical composition for use according to any one of additional example embodiments 65 to 73, wherein the amount of the compound of Formula I or pharmaceutically acceptable salt thereof is an amount that is less than the amount of brimonidine needed to achieve the same therapeutic effects.

Additional example embodiment 76: The pharmaceutical composition for use according to any one of additional example embodiments 65 to 73, wherein the pharmaceutical composition, when administered to the individual, causes an amount of reduction in pupil size such that the pupil is constricted to a size of between 2 and 3 mm.

Additional example embodiment 77: The pharmaceutical composition for use according to any one of additional example embodiments 65 to 73, wherein the pharmaceutical composition, when administered to the individual, causes an amount of reduction in pupil size such that the pupil is constricted to a size of 3 mm or less.

Additional example embodiment 78: The pharmaceutical composition for use according to any one of additional example embodiments 65 to 73, wherein the pharmaceutical composition, when administered to the individual, causes an amount of reduction in pupil size such that the pupil is constricted to a size of 2.5 mm or less.

Additional example embodiment 79: The pharmaceutical composition for use according to any one of additional example embodiment 65 to 73, wherein the pharmaceutical composition, when administered to the individual, causes an improvement in near visual acuity.

Additional example embodiment 80: The pharmaceutical composition for use according to any one of additional example embodiments 65 to 73, wherein the pharmaceutical composition, when administered to the individual, causes an improvement in intermediate visual acuity.

Additional example embodiment 81: The pharmaceutical composition for use according to any one of additional example embodiments 65 to 73, wherein the pharmaceutical composition, when administered to the individual, causes an improvement in distance visual acuity.

Additional example embodiment 82: The pharmaceutical composition for use according to any one of additional example embodiments 79 to 81, wherein the improvement in visual acuity is an at least 2-line improvement.

Additional example embodiment 83: The pharmaceutical composition for use according to any one of additional example embodiments 79 to 81, wherein the improvement in visual acuity is an at least 3-line improvement.

Additional example embodiment 84: The pharmaceutical composition for use according to any one of additional example embodiments 76 to 83, wherein the reduction in pupil size or improvement in visual acuity is maintained for at least 1 hour.

Additional example embodiment 85: The pharmaceutical composition for use according to any one of additional example embodiments 76 to 83, wherein the reduction in pupil size or improvement in visual acuity is maintained for at least 2 hours.

Additional example embodiment 86: The pharmaceutical composition for use according to any one of additional example embodiments 76 to 83, wherein the reduction in pupil size or improvement in visual acuity is maintained for at least 4 hours.

Additional example embodiment 87: The pharmaceutical composition for use according to any one of additional example embodiments 76 to 83, wherein the reduction in pupil size or improvement in visual acuity is maintained for at least 6 hours.

Additional example embodiment 88: The pharmaceutical composition for use according to any one of additional example embodiments 76 to 83, wherein the reduction in pupil size or improvement in visual acuity is maintained for at least 9 hours.

Additional example embodiment 89: The pharmaceutical composition for use according to any one of additional example embodiments 76 to 83, wherein the reduction in pupil size or improvement in visual acuity is maintained for at least 10 hours.

Additional example embodiment 90: The pharmaceutical composition for use according to any one of additional example embodiments 76 to 83, wherein the reduction in pupil size or improvement in visual acuity is maintained for at least 12 hours.

Additional example embodiment 91: The pharmaceutical composition for use according to any one of additional example embodiments 76 to 90, wherein the reduction in pupil size or improvement in visual acuity is achieved when the individual is exposed to luminance levels of less than 200 cd/m².

Additional example embodiment 92: The pharmaceutical composition for use according to any one of additional example embodiments 76 to 90, wherein the reduction in pupil size or improvement in visual acuity is achieved when the individual is exposed to luminance levels of less than 150 cd/m².

Additional example embodiment 93: The pharmaceutical composition for use according to any one of additional example embodiments 76 to 90, wherein the reduction in pupil size or improvement in visual acuity is achieved when the individual is exposed to luminance levels of less than 100 cd/m².

Additional example embodiment 94: The pharmaceutical composition for use according to any one of additional example embodiments 76 to 90, wherein the reduction in pupil size or improvement in visual acuity is achieved when the individual is exposed to luminance levels of less than 50 cd/m².

Additional example embodiment 95: The pharmaceutical composition for use according to any one of additional example embodiments 76 to 90, wherein the reduction in pupil size or improvement in visual acuity is achieved when the individual is exposed to luminance levels of less than 10 cd/m².

Additional example embodiment 96: The pharmaceutical composition for use according to any one of additional example embodiments 76 to 90, wherein the reduction in pupil size or improvement in visual acuity is achieved when the individual is exposed to luminance levels of less than 5 cd/m².

Additional example embodiment 97: The pharmaceutical composition for use according to any one of additional example embodiments 76 to 90, wherein the reduction in pupil size or improvement in visual acuity is achieved when the individual is exposed to luminance levels of less than 2 cd/m².

Additional example embodiment 98: Use of the pharmaceutical composition of any one of additional example embodiments 1 to 31 in a method of treating an ocular condition in an individual in need thereof, the method comprising administering the pharmaceutical composition to the individual, and wherein the ocular condition is selected from the group consisting of presbyopia, poor night vision, visual glare, visual starbursts, visual halos, and night myopia.

Additional example embodiment 99: The use according to additional example embodiment 98, wherein the ocular condition is presbyopia.

Additional example embodiment 100: The use according to additional example embodiment 98, wherein the ocular condition is poor night vision.

Additional example embodiment 101: The use according to additional example embodiment 98, wherein the ocular condition is visual glare.

Additional example embodiment 102: The use according to additional example embodiment 98, wherein the ocular condition is visual starbursts.

Additional example embodiment 103: The use according to additional example embodiment 98, wherein the ocular condition is visual halos.

Additional example embodiment 104: The use according to additional example embodiment 98, wherein the ocular condition is night myopia.

Additional example embodiment 105: The use according to any one of additional example embodiments 98 to 104, wherein the pharmaceutical composition is administered to one or both eyes of the individual.

Additional example embodiment 106: The use according to additional example embodiment 105, wherein the administration to the eye is topical administration.

Additional example embodiment 107: The use according to any one of additional example embodiments 98 to 106, wherein the amount of the compound of Formula I or pharmaceutically acceptable salt thereof, when administered to the individual, has binding to the iris pigment that is less than the binding to the iris pigment exhibited by brimonidine.

Additional example embodiment 108: The use according to any one of additional example embodiments 98 to 106, wherein the amount of the compound of Formula I or pharmaceutically acceptable salt thereof is an amount that is less than the amount of brimonidine needed to achieve the same therapeutic effects.

Additional example embodiment 109: The use according to any one of additional example embodiments 98 to 106, wherein the pharmaceutical composition, when administered to the individual, causes an amount of reduction in pupil size such that the pupil is constricted to a size of between 2 and 3 mm.

Additional example embodiment 110: The use according to any one of additional example embodiments 98 to 106, wherein the pharmaceutical composition, when administered to the individual, causes an amount of reduction in pupil size such that the pupil is constricted to a size of 3 mm or less.

Additional example embodiment 111: The use according to any one of additional example embodiments 98 to 106, wherein the pharmaceutical composition, when administered to the individual, causes an amount of reduction in pupil size such that the pupil is constricted to a size of 2.5 mm or less.

Additional example embodiment 112: The use according to any one of additional example embodiments 98 to 106, wherein the pharmaceutical composition, when administered to the individual, causes an improvement in near visual acuity.

Additional example embodiment 113: The use according to any one of additional example embodiments 98 to 106, wherein the pharmaceutical composition, when administered to the individual, causes an improvement in intermediate visual acuity.

Additional example embodiment 114: The use according to any one of additional example embodiments 98 to 106, wherein the pharmaceutical composition, when administered to the individual, causes an improvement in distance visual acuity.

Additional example embodiment 115: The use according to any one of additional example embodiments 112 to 114, where in the improvement in visual acuity is an at least 2-line improvement.

Additional example embodiment 116: The use according to any one of additional example embodiments 112 to 114, where in the improvement in visual acuity is an at least 3-line improvement.

Additional example embodiment 117: The use according to any one of additional example embodiments 109 to 116, wherein the reduction in pupil size or improvement in visual acuity is maintained for at least 1 hour.

Additional example embodiment 118: The use according to any one of additional example embodiments 109 to 114, wherein the reduction in pupil size or improvement in visual acuity is maintained for at least 2 hours.

Additional example embodiment 119: The use according to any one of additional example embodiments 109 to 114, wherein the reduction in pupil size or improvement in visual acuity is maintained for at least 4 hours.

Additional example embodiment 120: The use according to any one of additional example embodiments 109 to 114, wherein the reduction in pupil size or improvement in visual acuity is maintained for at least 6 hours.

Additional example embodiment 121: The use according to any one of additional example embodiments 109 to 114, wherein the reduction in pupil size or improvement in visual acuity is maintained for at least 9 hours.

Additional example embodiment 122: The use according to any one of additional example embodiments 109 to 114, wherein the reduction in pupil size or improvement in visual acuity is maintained for at least 10 hours.

Additional example embodiment 123: The use according to any one of additional example embodiments 109 to 114, wherein the reduction in pupil size or improvement in visual acuity is maintained for at least 12 hours.

Additional example embodiment 124: The use according to any one of additional example embodiments 109 to 123, wherein the reduction in pupil size or improvement in visual acuity is achieved when the individual is exposed to luminance levels of less than 200 cd/m².

Additional example embodiment 125: The use according to any one of additional example embodiments 102 to 123, wherein the reduction in pupil size or improvement in visual acuity is achieved when the individual is exposed to luminance levels of less than 150 cd/m².

Additional example embodiment 126: The use according to any one of additional example embodiments 102 to 123, wherein the reduction in pupil size or improvement in visual acuity is achieved when the individual is exposed to luminance levels of less than 100 cd/m².

Additional example embodiment 127: The use according to any one of additional example embodiments 102 to 123, wherein the reduction in pupil size or improvement in visual acuity is achieved when the individual is exposed to luminance levels of less than 50 cd/m².

Additional example embodiment 128: The use according to any one of additional example embodiments 102 to 123, wherein the reduction in pupil size or improvement in visual acuity is achieved when the individual is exposed to luminance levels of less than 10 cd/m².

Additional example embodiment 129: The use according to any one of additional example embodiments 102 to 123, wherein the reduction in pupil size or improvement in visual acuity is achieved when the individual is exposed to luminance levels of less than 5 cd/m².

Additional example embodiment 130: The use according to any one of additional example embodiments 102 to 123, wherein the reduction in pupil size or improvement in visual acuity is achieved when the individual is exposed to luminance levels of less than 2 cd/m².

Additional example embodiment 131: Use of a compound of Formula I:

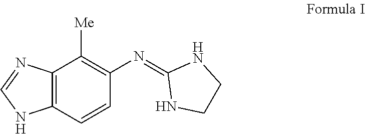 [SMILES: Cc1c(N=C2NCCN2)ccc2[nH]cnc12]

or a pharmaceutically acceptable salt thereof, in the manufacture of a medicament for the treatment of an ocular condition in an individual in need thereof, wherein the medicament is a pharmaceutical composition according to any one of additional example embodiments 1 to 31, and wherein the ocular condition is selected from the group consisting of presbyopia, poor night vision, visual glare, visual starbursts, visual halos, and night myopia.

Additional example embodiment 132: The use according to additional example embodiment 131, wherein the ocular condition is presbyopia.

Additional example embodiment 133: The use according to additional example embodiment 131, wherein the ocular condition is poor night vision.

Additional example embodiment 134: The use according to additional example embodiment 131, wherein the ocular condition is visual glare.

Additional example embodiment 135: The use according to additional example embodiment 131, wherein the ocular condition is visual starbursts.

Additional example embodiment 136: The use according to additional example embodiment 131, wherein the ocular condition is visual halos.

Additional example embodiment 137: The use according to additional example embodiment 131, wherein the ocular condition is night myopia.

Additional example embodiment 138: The use according to any one of additional example embodiments 131-137, wherein the medicament, when administered to the individual, is administered to one or both eyes of the individual.

Additional example embodiment 139: The use according to additional example embodiment 138, wherein the administration to the eye is topical administration.

Additional example embodiment 140: The use according to any one of additional example embodiments 131 to 139, wherein the amount of the compound of Formula I or pharmaceutically acceptable salt thereof in the medicament, when administered to the individual, has binding to the iris pigment that is less than the binding to the iris pigment exhibited by brimonidine.

Additional example embodiment 141: The use according to any one of additional example embodiments 131 to 139, wherein the amount of the compound of Formula I or pharmaceutically acceptable salt thereof in the medicament is an amount that is less than the amount of brimonidine needed to achieve the same therapeutic effects.

Additional example embodiment 142: The use according to any one of additional example embodiments 131 to 139, wherein the medicament, when administered to the individual, causes an amount of reduction in pupil size such that the pupil is constricted to a size of between 2 and 3 mm.

Additional example embodiment 143: The use according to any one of additional example embodiments 131 to 139, wherein the medicament, when administered to the individual, causes an amount of reduction in pupil size such that the pupil is constricted to a size of 3 mm or less.

Additional example embodiment 144: The use according to any one of additional example embodiments 131 to 139, wherein the medicament, when administered to the individual, causes an amount of reduction in pupil size such that the pupil is constricted to a size of 2.5 mm or less.

Additional example embodiment 145: The use according to any one of additional example embodiments 131 to 139, wherein the medicament, when administered to the individual, causes an improvement in near visual acuity.

Additional example embodiment 146: The use according to any one of additional example embodiments 131 to 139, wherein the medicament, when administered to the individual, causes an improvement in intermediate visual acuity.

Additional example embodiment 147: The use according to any one of additional example embodiments 131 to 139, wherein the medicament, when administered to the individual, causes an improvement in distance visual acuity.

Additional example embodiment 148: The use according to any one of additional example embodiments 145 to 147, where in the improvement in visual acuity is an at least 2-line improvement.

Additional example embodiment 149: The use according to any one of additional example embodiments 145 to 147, where in the improvement in visual acuity is an at least 3-line improvement.

Additional example embodiment 150: The use according to any one of additional example embodiments 142 to 149, wherein the reduction in pupil size or improvement in visual acuity is maintained for at least 1 hour.

Additional example embodiment 151: The use according to any one of additional example embodiments 142 to 149, wherein the reduction in pupil size or improvement in visual acuity is maintained for at least 2 hours.

Additional example embodiment 152: The use according to any one of additional example embodiments 142 to 149, wherein the reduction in pupil size or improvement in visual acuity is maintained for at least 4 hours.

Additional example embodiment 153: The use according to any one of additional example embodiments 142 to 149, wherein the reduction in pupil size or improvement in visual acuity is maintained for at least 6 hours.

Additional example embodiment 154: The use according to any one of additional example embodiments 142 to 149, wherein the reduction in pupil size or improvement in visual acuity is maintained for at least 9 hours.

Additional example embodiment 155: The use according to any one of additional example embodiments 142 to 149, wherein the reduction in pupil size or improvement in visual acuity is maintained for at least 10 hours.

Additional example embodiment 156: The use according to any one of additional example embodiments 142 to 149, wherein the reduction in pupil size or improvement in visual acuity is maintained for at least 12 hours.

Additional example embodiment 157: The use according to any one of additional example embodiments 142 to 156, wherein the reduction in pupil size or improvement in visual acuity is achieved when the individual is exposed to luminance levels of less than 200 cd/m².

Additional example embodiment 158: The use according to any one of additional example embodiments 142 to 156, wherein the reduction in pupil size or improvement in visual acuity is achieved when the individual is exposed to luminance levels of less than 150 cd/m².

Additional example embodiment 159: The use according to any one of additional example embodiments 142 to 156, wherein the reduction in pupil size or improvement in visual acuity is achieved when the individual is exposed to luminance levels of less than 100 cd/m².

Additional example embodiment 160: The use according to any one of additional example embodiments 142 to 156, wherein the reduction in pupil size or improvement in visual acuity is achieved when the individual is exposed to luminance levels of less than 50 cd/m².

Additional example embodiment 161: The use according to any one of additional example embodiments 142 to 156, wherein the reduction in pupil size or improvement in visual acuity is achieved when the individual is exposed to luminance levels of less than 10 cd/m².

Additional example embodiment 162: The use according to any one of additional example embodiments 142 to 156, wherein the reduction in pupil size or improvement in visual acuity is achieved when the individual is exposed to luminance levels of less than 5 cd/m².

Additional example embodiment 163: The use according to any one of additional example embodiments 142 to 156, wherein the reduction in pupil size or improvement in visual acuity is achieved when the individual is exposed to luminance levels of less than 2 cd/m².

Additional example embodiment 164: A pharmaceutical composition substantially as described herein.

Additional example embodiment 165: A pharmaceutical composition comprising a compound of Formula I:

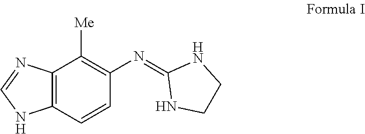 [SMILES: Cc1c(N=C2NCCN2)ccc2[nH]cnc12]

or a pharmaceutically acceptable salt thereof, a buffer, sodium chloride, and water substantially as described herein.

Additional example embodiment 166: A method of treating an ocular condition selected from the group consisting of presbyopia, poor night vision, visual glare, visual starbursts, visual halos, and night myopia substantially as described herein.

Additional example embodiment 167: A method of treating an ocular condition selected from the group consisting of presbyopia, poor night vision, visual glare, visual starbursts, visual halos, and night myopia with a pharmaceutical composition substantially as described herein.

Additional example embodiment 168: A method of using pharmaceutical composition comprising a compound of Formula I:

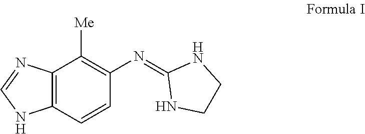 [SMILES: Cc1c(N=C2NCCN2)ccc2[nH]cnc12]

or a pharmaceutically acceptable salt thereof, a buffer, sodium chloride, and water substantially as described herein.

Additional example embodiment 169: The method according to any one of additional example embodiments 32 to 64, wherein the individual also is in need of reducing redness of an eye or increasing whiteness of an eye.

Additional example embodiment 170: The method according to additional example embodiment 169, wherein administering to the individual the therapeutically effective amount of the pharmaceutical composition also results in reducing redness of an eye or increasing whiteness of an eye in the individual.

Additional example embodiment 171: The pharmaceutical composition for use according to any one of additional example embodiments 65 to 97, wherein the individual also is in need of reducing redness of an eye or increasing whiteness of an eye.

Additional example embodiment 172: The pharmaceutical composition for use according to additional example embodiment 171, wherein administering to the individual the pharmaceutical composition also results in reducing redness of an eye or increasing whiteness of an eye in the individual.

Additional example embodiment 173: The use according to any one of additional example embodiments 98 to 130, wherein the individual also is in need of reducing redness of an eye or increasing whiteness of an eye.

Additional example embodiment 174: The pharmaceutical composition for use according to additional example embodiment 173, wherein administering to the individual the pharmaceutical composition also results in reducing redness of an eye or increasing whiteness of an eye in the individual.

Additional example embodiment 175: The use according to any one of additional example embodiments 131 to 163, wherein the individual also is in need of reducing redness of an eye or increasing whiteness of an eye.

Additional example embodiment 176: The pharmaceutical composition for use according to additional example embodiment 175, wherein the medicament, when administered to the individual, also results in reducing redness of an eye or increasing whiteness of an eye in the individual.

These and other embodiments and aspects of the disclosure are described in more detail herein.

### DESCRIPTION OF THE DRAWINGS

FIG. 1 shows a plot of hyperemia reduction in rabbits treated with the compound of Formula I (Compound 1) and LUMIFY® on the investigator (clinical) scale.

FIG. 2 shows a plot of hyperemia reduction in rabbits treated with the compound of Formula I (Compound 1) and LUMIFY® on the modified Hackett-McDonald (HM) scoring scale (veterinary).

FIG. 3 shows a plot of hyperemia reduction in rabbits treated with the compound of Formula I (Compound 1) and LUMIFY® on a digital redness scale.

FIG. 4 shows a plot of the dose miotic response curve in Dutch Belted rabbits when topically dosed with compound 2 (see Example 1). Percentage amounts are % w:v.

FIG. 5 shows a plot of the dose miotic response curve in Dutch Belted rabbits when topically dosed with brimonidine (compound 4; see Example 1). Percentage amounts are % w:v.

FIG. 6 shows a plot of the dose miotic response curve in Dutch Belted rabbits when topically dosed with the compound of Formula I (compound 1; see Example 1). Percentage amounts are % w:v.

FIG. 7 shows a responder analysis of subjects (rabbits) with >2.5 mm pupil change when dosed with the compound of Formula I (compound 1) or brimonidine (compound 4), both at 0.1% w:v.

FIG. 8 shows a comparison of brimonidine (compound 4) and the compound of Formula I (compound 1: (see Example 1) duration of miotic action after topical dosing in DB rabbits under room light condition. Percentage amounts are % w:v.

FIG. 9 shows a plot of the dose miotic response curve (over 9 hours) in Dutch Belted rabbits when topically dosed with the compound of Formula I (compound 1; see Example 1). Percentage amounts are % w:v.

FIG. 10 shows a plot of the comparison of dose miotic response curves in Dutch Belted rabbits when topically dosed with the compound of Formula I (compound 1; see Example 1) or with compound 3 (see Example 1). Percentage amounts are % w:v.

### DETAILED DESCRIPTION

It is to be understood that both the foregoing general description and the following detailed description are exemplary and explanatory only and are not restrictive of the invention claimed. As used herein, the use of the singular includes the plural unless specifically stated otherwise. As used herein, “or” means “and/or” unless stated otherwise. Furthermore, use of the term “including” as well as other forms, such as “includes,” and “included,” is not limiting. The section headings used herein are for organizational purposes only and are not to be construed as limiting the subject matter described.

Unless specific definitions are provided, the nomenclatures utilized in connection with the laboratory procedures and techniques of analytical chemistry, synthetic organic and inorganic chemistry described herein are those known in the art. Standard chemical symbols are used interchangeably with the full names represented by such symbols. Thus, for example, the terms “hydrogen” and “H” are understood to have identical meaning, as are “methyl,” “Me”, and “CH₃”. Standard techniques can be used for chemical syntheses, chemical analyses, and formulation.

In some embodiments, the compounds described herein (such as the compound of Formula I) can include pharmaceutically acceptable salts thereof. Such salts can include, for example, acid addition salts, such as hydrochloride, hydrobromide, sulfate, nitrate, phosphate, phosphorate, acetate, propionate, glycolate, pyruvate, oxalate, malate, malonate, succinate, maleate, fumarate, tartrate, citrate, benzoate, cinnamate, mandelate, methanesulfonate, ethanesulfonate, p-toluene-sulfonate, salicylate and the like, and base addition salts, such as sodium, potassium, calcium, magnesium, lithium, aluminum, zinc, ammonium, ethylenediamine, arginine, piperazine and the like, as well as others identifiable to a skilled person upon a reading of the present disclosure (see, e.g., Handbook of Pharmaceutical Salts, P. Heinrich Stahl & Camille G. Wermuth (Eds), Verlag; Helvetica Chimica Acta-Zürich, 2002, 329-345; and Berge et al., Journal of Pharmaceutical Science, 1977, 66:1-19).

Certain compounds described herein can exist as tautomers which can interconvert between themselves. The structural depiction herein of a particular tautomer should not be construed as limiting the compound to the particular tautomer depicted (even if it may not be the predominant tautomer under a particular set of conditions) unless otherwise indicated.

Unless indicated otherwise herein, the term “about” when used in reference to a value (e.g., weight percentages) is intended to include values proximate to the recited value (and/or range of values) that are equivalent (e.g. bioequivalent) in terms of the functionality of the individual ingredient (e.g. active ingredient or excipient), the composition, or the embodiment. Furthermore, as will be understood by a skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements and that some values and amounts can be rounded up or down such that they would be “about the same” as another value or amount.

The term “therapeutically effective amount” refers to an amount that is effective, when administered to an individual in need of treatment of an ocular condition, such as human or non-human patient, to treat the ocular condition (e.g., inducing ocular vasoconstriction, reducing ocular hyperemia, reducing eye redness, and/or increasing eye whiteness). The extent and/or success of the treatment of the ocular condition when a therapeutically effective amount of a compound and/or composition is administered to an individual would be readily identifiable to a skilled person as is described herein.

The term “compound of Formula I” refers to the compound having the following chemical structure:

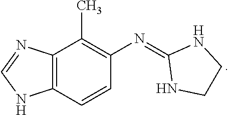 [SMILES: Cc1c(N=C2NCCN2)ccc2[nH]cnc12]

In one embodiment, the method comprises administering to the individual a therapeutically effective amount of a compound of Formula I or a pharmaceutically acceptable salt thereof. The compound of Formula I can be synthesized by methods known to persons skilled in the art, such as the methods described in U.S. Pat. Nos. 6,495,583 and 5,478,858.

In some embodiments, the method comprises administering to the individual a therapeutically effective amount of a pharmaceutical compositing comprising a compound of Formula I or a pharmaceutically acceptable salt thereof, and a pharmaceutically acceptable excipient.

In some embodiments the disclosure provides methods for reducing redness of an eye of an individual by administering to the eye of the individual a therapeutically effective amount of the compound of Formula I or a pharmaceutically acceptable salt thereof. In some embodiments the disclosure provides methods of reducing redness of an eye of an individual by administering to the eye of the individual a therapeutically effective amount of a pharmaceutical composition comprising the compound of Formula I or a pharmaceutically acceptable salt thereof, and a pharmaceutically acceptable excipient. The methods reduce eye redness which is naturally occurring or which is due to an eye pathology. Exemplary causes of eye redness include allergies (e.g., caused by pollen, dust mites, molds, pet dander), irritants (e.g., smoke, dust particles, chemical vapors), foreign objects (e.g., eyelash), digital eye strain (e.g., caused by looking at a phone, computer, or tablet for an extended period of time), contact lens wear, dry eye, high stress levels, drug use (e.g., cannabis, alcohol, illicit drugs) and the like. Other causes of eye redness include, for example, laser-assisted in situ keratomileusis (LASIK) eye surgery, styes, blocked tear ducts, blepharitis, infection, inflammation (such as uveitis), injury (surgical or non-surgical, such as corneal abrasion), corneal ulcer, sub-conjunctival hemorrhage, and the like. In some aspects, the compounds or compositions are administered to the eye of the individual after the individual undergoes LASIK eye surgery in order to reduce redness of the eye. In some aspects, the compounds or compositions are administered to the eye of the individual within 24 hours after the individual undergoes LASIK eye surgery in order to reduce redness of the eye. The term “eye pathology” refers to the “causes of eye redness” described herein or any other causes of eye redness known in the art. In some aspects, eye redness can be naturally occurring in an individual without an eye pathology. In some aspects, the redness of the eye is chronic redness of the eye.

In some embodiments, the disclosure provides methods of reducing redness of an eye caused by minor eye irritations in the eye of the individual by administering to the eye of the individual a therapeutically effective amount of the compound of Formula I or a pharmaceutically acceptable salt thereof. In some embodiments, the disclosure provides methods of reducing redness of an eye caused by minor eye irritations in the eye of the individual by administering to the eye of the individual a therapeutically effective amount of a pharmaceutical composition comprising the compound of Formula I or a pharmaceutically acceptable salt thereof, and a pharmaceutically acceptable excipient. Minor eye irritations can be caused by allergies (e.g., caused by pollen, dust mites, molds, pet dander), irritants (e.g., smoke, dust particles, chemical vapors), foreign objects (e.g., eyelash), digital eye strain (e.g., caused by looking at a phone, computer, or tablet for an extended period of time), dry eye, high stress levels, drug use (e.g., cannabis, alcohol, illicit drugs) and the like. In some aspects, the minor eye irritation is caused by an allergy. In some aspects, the minor eye irritation is caused by an irritant. In some aspects, the minor eye irritation is caused by a foreign object. In some aspects, the eye redness is caused by infection. In some aspects, the eye redness is caused by inflammation. In some aspects, the eye redness is caused by eye surgery. In some aspects, the minor eye irritation is caused by digital eye strain. In some aspects, the minor eye irritation is caused by dry eye. In some aspects, the minor eye irritation is caused by drug use (e.g., cannabis, alcohol, illicit drugs). In some aspects, the redness of the eye caused by minor eye irritation is chronic redness of the eye.

In some embodiments, the disclosure provides methods of reducing redness of an eye due to dilation of vessels of the conjunctiva, the episclera, the sclera, or a combination of two or more thereof by administering to the eye of the individual a therapeutically effective amount of the compound of Formula I or a pharmaceutically acceptable salt thereof. In some embodiments, the disclosure provides methods of reducing redness of an eye due to dilation of vessels of the conjunctiva, the episclera, the sclera, or a combination of two or more thereof by administering to the eye of the individual a therapeutically effective amount of a pharmaceutical composition comprising the compound of Formula I or a pharmaceutically acceptable salt thereof, and a pharmaceutically acceptable excipient. In some aspects, the methods are for reducing redness of an eye caused by dilation of vessels of the conjunctiva. In some aspects, the methods are for reducing redness of an eye caused by dilation of vessels of the episclera. In some aspects, the methods are for reducing redness of an eye caused by dilation of vessels of the sclera. In some aspects, the methods are for reducing redness of an eye caused by infection. In some aspects, the methods are for reducing redness of an eye caused by inflammation. In some aspects, the methods are for reducing redness of an eye caused by eye surgery.

In some embodiments, the disclosure provides methods for increasing the whiteness of an eye of an individual by administering to the eye of the individual a therapeutically effective amount of the compound of Formula I or a pharmaceutically acceptable salt thereof. In some embodiments, the disclosure provides methods for increasing the whiteness of an eye of an individual by administering to the eye of the individual a therapeutically effective amount of a pharmaceutical composition comprising the compound of Formula I or a pharmaceutically acceptable salt thereof, and a pharmaceutically acceptable excipient. The methods can increase whiteness of an eye one or more shades beyond the baseline whiteness of the eye. This increase in whiteness may be important for cosmetic or other reasons. A normal healthy eye has a certain baseline level of whiteness, which slightly varies from person to person. Reduced whiteness of the sclera is often viewed as cosmetically less desirable, and may be an indicator of fatigue, age, lack of sleep, drug or alcohol use, emotional lability, and overall poor health. Whiter sclera is often viewed as more cosmetically desirable, associated with improved hygiene and/or health, and a cleaner, healthier lifestyle. Thus, an individual may want to whiten their eyes for purely aesthetic reasons. In some aspects, the methods are for increasing the whiteness of an eye of an individual who does not have an eye pathology. In some aspects, the methods are for increasing the whiteness of an eye of an individual for aesthetic reasons.

In some embodiments, the disclosure provides methods for reducing the redness of an eye and increasing the whiteness of an eye of an individual by administering to the eye of the individual a therapeutically effective amount of the compound of Formula I or a pharmaceutically acceptable salt thereof. In some embodiments the disclosure provides methods for reducing the redness of an eye and increasing the whiteness of an eye of an individual by administering to the eye of the individual a therapeutically effective amount of a pharmaceutical composition comprising the compound of Formula I or a pharmaceutically acceptable salt thereof, and a pharmaceutically acceptable excipient. Thus the compound of Formula I can be administered to the eye of an individual to simultaneously reduce the redness of an eye and increase the whiteness of an eye.

The compounds and compositions described herein can reduce redness of the eye and/or increase whiteness of the eye from about 1 second to about 1 minute after administration to the eye. In some aspects, compounds and compositions described herein reduce redness of the eye and/or increase whiteness of the eye from about 5 seconds to about 1 minute after administration to the eye. In some aspects, compounds and compositions described herein reduce redness of the eye and/or increase whiteness of the eye from about 10 seconds to about 1 minute after administration to the eye. In some aspects, compounds and compositions described herein reduce redness of the eye and/or increase whiteness of the eye from about 15 seconds to about 1 minute after administration to the eye. In some aspects, compounds and compositions described herein reduce redness of the eye and/or increase whiteness of the eye from about 20 seconds to about 1 minute after administration to the eye. In some aspects, compounds and compositions described herein reduce redness of the eye and/or increase whiteness of the eye from about 25 seconds to about 1 minute after administration to the eye. In some aspects, compounds and compositions described herein reduce redness of the eye and/or increase whiteness of the eye from about 30 seconds to about 1 minute after administration to the eye. In some aspects, compounds and compositions described herein reduce redness of the eye and/or increase whiteness of the eye from about 40 seconds to about 1 minute after administration to the eye. In some aspects, compounds and compositions described herein reduce redness of the eye and/or increase whiteness of the eye from about 45 seconds to about 1 minute after administration to the eye. In some aspects, compounds and compositions described herein reduce redness of the eye and/or increase whiteness of the eye in about 5 seconds after administration to the eye. In some aspects, compounds and compositions described herein reduce redness of the eye and/or increase whiteness of the eye in about 10 seconds after administration to the eye. In some aspects, compounds and compositions described herein reduce redness of the eye and/or increase whiteness of the eye in about 15 seconds after administration to the eye. In some aspects, compounds and compositions described herein reduce redness of the eye and/or increase whiteness of the eye in about 20 seconds after administration to the eye. In some aspects, compounds and compositions described herein reduce redness of the eye and/or increase whiteness of the eye in about 30 seconds after administration to the eye. In some aspects, compounds and compositions described herein reduce redness of the eye and/or increase whiteness of the eye in about 40 seconds after administration to the eye. In some aspects, compounds and compositions described herein reduce redness of the eye and/or increase whiteness of the eye in about 45 seconds after administration to the eye. In some aspects, compounds and compositions described herein reduce redness of the eye and/or increase whiteness of the eye in about 1 minute after administration to the eye. In some embodiments, the compounds and compositions described herein can cause a greater reduction in redness and/or increase in whiteness of the eye within the aforementioned time periods when compared to the administration of a second compound or composition (e.g. brimonidine or pilocarpine). For example, the compounds and compositions described herein can cause a greater reduction in redness at 1 minute after administration when compared to the administration of a second compound or composition (e.g. brimonidine or pilocarpine) which would not show as much of a reduction in redness at 1 minute after administration.

The compounds and compositions described herein can reduce redness of the eye and/or increase whiteness of the eye for at least 8 hours after administration to the eye. The compounds and compositions described herein can reduce redness of the eye and/or increase whiteness of the eye for at least 10 hours after administration to the eye. The compounds and compositions described herein can reduce redness of the eye and/or increase whiteness of the eye for at least 12 hours after administration to the eye. The compounds and compositions described herein can reduce redness of the eye and/or increase whiteness of the eye for at least 24 hours after administration to the eye. The compounds and compositions described herein can reduce redness of the eye and/or increase whiteness of the eye for about 4 hours to about 24 hours after administration to the eye, and for time periods in between those ranges. The compounds and compositions described herein can reduce redness of the eye and/or increase whiteness of the eye for about 6 hours to about 24 hours after administration to the eye, and for time periods in between those ranges. The compounds and compositions described herein can reduce redness of the eye and/or increase whiteness of the eye for about 8 hours to about 24 hours after administration to the eye, and for time periods in between those ranges. The compounds and compositions described herein can reduce redness of the eye and/or increase whiteness of the eye for about 9 hours to about 24 hours after administration to the eye, and for time periods in between those ranges. The compounds and compositions described herein can reduce redness of the eye and/or increase whiteness of the eye for about 10 hours to about 24 hours after administration to the eye, and for time periods in between those ranges. The compounds and compositions described herein can reduce redness of the eye and/or increase whiteness of the eye for about 11 hours to about 24 hours after administration to the eye, and for time periods in between those ranges. The compounds and compositions described herein can reduce redness of the eye and/or increase whiteness of the eye for about 12 hours to about 24 hours after administration to the eye, and for time periods in between those ranges. The compounds and compositions described herein can reduce redness of the eye and/or increase whiteness of the eye for about 8 hours after administration to the eye. The compounds and compositions described herein can reduce redness of the eye and/or increase whiteness of the eye for about 9 hours after administration to the eye. The compounds and compositions described herein can reduce redness of the eye and/or increase whiteness of the eye for about 10 hours after administration to the eye. The compounds and compositions described herein can reduce redness of the eye and/or increase whiteness of the eye for about 11 hours after administration to the eye. The compounds and compositions described herein can reduce redness of the eye and/or increase whiteness of the eye for about 12 hours after administration to the eye. The compounds and compositions described herein can reduce redness of the eye and/or increase whiteness of the eye for about 24 hours after administration to the eye. In some embodiments, the compounds and compositions described herein can have a greater reduction in eye redness and/or increase in eye whiteness for the aforementioned durations when compared to the administration of a second compound or composition (e.g. brimonidine or pilocarpine). For example, the compounds and compositions described herein can show a greater reduction in eye redness 8 hours after administration when compared to the administration of a second compound or composition (e.g. brimonidine or pilocarpine), which would not show as much eye redness at 8 hours.

The reduction in redness and/or the increase in whiteness in the eye caused by the compounds, compositions, and methods described herein can be measured by scales known in the art. Exemplary scales to measure decreases in eye redness and/or increases in eye whiteness include, for example, the Modified Hackett-McDonald Scoring Scale; McMonnies/Chapman-Davies scale (MC-D); the Institute for Eye Research scale (IER, previously known as CCLRU scale); the Efron scale; and the validated bulbar redness scale (VBR) developed at the Centre for Contact Lens Research. (The Use of Fractal Analysis and Photometry to Estimate the Accuracy of Bulbar Redness Grading Scales, Marc M. Schuze et al, Investigative Ophthalmology and Visual Science, 49:1398-1406 (2008)). In addition, the redness scale described in U.S. Pat. No. 8,765,758 can also be used to evaluate decreases in eye redness and/or increases in eye whiteness caused by the compounds, compositions, and methods described herein. Other scales would be identifiable to a skilled person upon a reading of the present disclosure.

In one embodiment, the Modified Hackett-McDonald Scoring Scale is used to assess eye redness and/or whiteness, which can be used both with humans and in a veterinary context. The scale is shown in Table 1 below.

TABLE 1
Modified Hackett-McDonald Scoring Scale

| | |
|---|---|
| Score | Description |

Conjunctival Congestion (Hyperemia)

| | |
|---|---|
| 0 | Normal, may appear blanched to reddish pink without perilimbal |
| | injection (except at the 12 and 6 o'clock positions) with |
| | vessels of the palpebral and bulbar conjunctiva easily observed |
| +1 | Mild, Slight; vessels definitely injected above normal. |
| | Predominantly confined to the palpebral conjunctival. |
| +2 | Moderate; more diffuse, deeper crimson red with perilimbal |
| | injection covering at least 75% of the circumference of the |
| | perilimbal region., individual vessels not easily discernible. |
| +3 | Severe; diffuse beefy red with congestion of both the bulbar and |
| | the palpebral conjunctiva along with pronounced perilimbal |
| | injection. Petechiae may be present on the nictitating membrane |
| | and/or the upper palpebral conjunctiva. |

Discomfort

| | |
|---|---|
| 0 | Normal |
| +1 | Slight; characterized by intermittent blinking and/or squinting. |
| +2 | Mild; characterized by repeated blinking and/or squinting; |
| | partial closure of the eye may be observed. |
| +3 | Moderate; characterized by repeated blinking and/or squinting |
| | with partial to complete closure of the eye; pawing at the eye |
| | usually observed. |
| +4 | Severe; characterized by firm closure of the eye for prolonged |
| | periods of time with repeated pawing |

Conjunctival Swelling (Chemosis)

| | |
|---|---|
| 0 | Normal or no swelling of the conjunctival tissue |
| +1 | Slight; any swelling above normal. |
| +2 | Mild; obvious swelling with partial eversion of the lid. |
| +3 | Moderate; swelling with lids about half closed. |
| +4 | Severe; swelling with lids half to fully closed. |

Discharge

| | |
|---|---|
| 0 | No discharge except may include a small amount of clear or |
| | mucoid material found in the medial canthus of a substantial |
| | number of animal eyes. |
| +1 | Slight; any amount of opaque mucoidal discharge different from |
| | normal |
| +2 | Moderate; opaque mucoidal discharge with moistening of the lid |
| | and the hairs just adjacent to the lid. |
| +3 | Severe; opaque mucoidal discharge with moistening of the lid |
| | and considerable area around the eye. |

The reduction in redness and/or the increase in whiteness can be measured qualitatively. For example, a medical professional or an individual receiving treatment can subjectively determine that the eye(s) has reduced redness and/or increased whiteness. Such determination can optionally be made based on a photograph prior to starting treatment with the compounds or compositions described herein, such that a before and after determination of reduced redness and/or increased whiteness can be qualitatively identified by a medical professional or an individual treated with the compounds and or compositions described herein.

In an embodiment, the qualitative measurement can be an ocular surface hyperemia (OSH) score, as shown in the Table 2 below.

TABLE 2

| Appearance | Score |
|---|---|
| Normal: normal eye color | 0 |
| Hi-Normal: slightly red eye, but predominantly | +0.5 |
| normal appearance | |
| Mild: number and diameter of red eye veins is | +1 |
| greater than normal | |
| Moderate: bright red eye veins, with increased | +2 |
| number and diameter of vessels, deeper crimson red | |
| Severe: prominent beefy red eye veins accompanied | +3 |
| by a reddish color of the conjunctiva | |

In some embodiments of the methods described herein, the compound of Formula I or a pharmaceutically acceptable salt thereof is administered to one eye or both eyes of the individual. In some embodiments, the compound of Formula I or a pharmaceutically acceptable salt thereof is administered to both eyes of the individual. In some embodiments, the compound of Formula I or a pharmaceutically acceptable salt thereof is administered to one eye of the individual.

In some embodiments of the methods described herein the compound of Formula I or a pharmaceutically acceptable salt thereof is topically administered to one or both eyes of the individual to reduce eye redness and/or increase eye whiteness. The composition comprising the compound of Formula I or a pharmaceutically acceptable salt thereof and a pharmaceutically excipient that is topically administered to the eye of the individual can be a liquid pharmaceutical composition, such as a solution, dispersion, emulsion, gel, and the like.

In some embodiments, the pharmaceutical composition comprising the compound of Formula I or the pharmaceutically acceptable salt thereof is a solution. In some aspects, the methods described herein comprise topically administering a solution comprising the compound of Formula I or a pharmaceutically acceptable salt thereof, and a pharmaceutically acceptable excipient to one or both eyes of the individual to reduce eye redness and/or increase eye whiteness.

In some embodiments of the methods described herein, the pharmaceutical composition comprising the compound of Formula I or the pharmaceutically acceptable salt thereof is a dispersion. In some aspects, the methods described herein comprise topically administering a dispersion comprising the compound of Formula I or a pharmaceutically acceptable salt thereof, and a pharmaceutically acceptable excipient to one or both eyes of the individual to reduce eye redness and/or increase eye whiteness.

When the compound of Formula (I) or the pharmaceutical composition (e.g., solution, dispersion) comprising the compound of Formula (I) is topically administered to one or both eyes of the individual, the methods comprise topically administering one drop of the pharmaceutical composition (e.g., solution, dispersion) to one eye or both eyes of the individual. In some aspects, the methods comprise topically administering two drops of the pharmaceutical composition (e.g., solution, dispersion) to one eye or both eyes of the individual. In some aspects, the methods comprise topically administering three drops of the pharmaceutical composition (e.g., solution, dispersion) to one eye or both eyes of the individual. In some aspects, the methods comprise topically administering one drop of a solution comprising the compound of Formula (I) or a pharmaceutically acceptable salt thereof to one eye or to both eyes of the individual to reduce redness or to increase whiteness. One “drop” of a solution, dispersion, or emulsion will be known to the skilled artisan. In some aspects, one “drop” is from about 0.01 ml to about 0.1 ml. In some aspects, one “drop” is from about 0.02 ml to about 0.08 ml. In some aspects, one “drop” is from about 0.03 ml to about 0.07 ml. In some aspects, one “drop” is from about 0.04 ml to about 0.06 ml. In some aspects, one “drop” is about 0.05 ml.

The inventors have surprisingly found the compound of Formula I has greater in vivo activity than would have been predicted based in the in vitro activity of the compound of Formula I when compared to similar alpha-2-adrenergic receptor agonists, which can result in a greater duration of therapeutic activity (e.g. inducing ocular vasoconstriction, reducing ocular hyperemia, reducing eye redness, and/or increasing eye whiteness) of the compound of Formula I when compared to other alpha-2-adrenergic receptor agonists. Thus, in some embodiments, the therapeutically effective amount of the compound of Formula I is an amount which, when administered to the individual, results in an increased efficacy (including onset of action) and/or duration of effect when compared to other alpha-2-adrenergic receptor agonists (for example, brimonidine).

The inventors have also surprisingly found that, unlike the marketed alpha-2-adrenergic receptor agonist brimonidine (which has elevated binding to iris melanin pigments), the compound of Formula I does not exhibit much binding to iris melanin pigments. Thus, the compound of Formula I can be administered with more consistent dosing between individuals having different eye colors/iris pigmentation (e.g. individuals in need and/or desire of ocular vasoconstriction, reducing ocular hyperemia, reducing eye redness, and/or increasing eye whiteness). Thus, in some embodiments, the therapeutically effective amount of the compound of Formula I is an amount which, when administered to the individual, results in reduced amount of binding to the iris pigment of the individual when compared to the administration of about the same amount of another alpha-2-adrenergic receptor agonist (for example, brimonidine). For example, in some embodiments, a particular therapeutically effective amount of the compound of Formula I, when administered to an individual, can result in binding to the iris pigment that is about 8 to about 10 times less than the binding to the iris pigment when about the same amount of brimonidine is administered to the individual, especially when the individual has an iris which would be considered a dark iris (see, e.g. Franssen et al, Journal of Optometry, 1(1):36-40 (2008)).

Furthermore, this reduced amount of binding to iris pigments can result in reduced amount of compound of Formula I needed to achieve a particular therapeutic effect than would be needed if brimonidine were used, especially when the individual has an iris which would be considered a dark iris. Thus, in some embodiments, the amount of compound of Formula I needed would be about 30 to about 100 times less than the amount of brimonidine needed to achieve similar therapeutic effects as brimonidine (e.g. inducing ocular vasoconstriction, reducing ocular hyperemia, reducing eye redness, and/or increasing eye whiteness). In some embodiments, the amount of compound of Formula I needed would be about 2 times, about 3 times, about 4 times, about 5 times, about 10 times, about 20 times, about 30 times, about 40 times, about 50 times, about 60 times, about 70 times, about 80 times, about 90 times, or about 100 times less than the amount of brimonidine needed to achieve similar therapeutic effects as brimonidine (e.g. reduced eye redness and/or increased eye whiteness).

In addition, due to the reduced amount of the compound of Formula I needed, it is anticipated that the lower potential compound of Formula I needed would result in reduced incidence of side effects normally associated with alpha-2-adrenergic receptor agonists (e.g. sedation). Accordingly, in some embodiments, pharmaceutical compositions described herein reduce the incidence of at least one adverse event selected from tachyphylaxis, rebound congestion, ocular hyperemia, eye irritation, increased intraocular pressure, eye pain, dizziness, fatigue, headache, hypotension, nasopharyngitis, sinusitis, bradycardia, iritis, miosis, skin reactions, erythema, eyelid pruritus, rash, vasodilation, tachycardia, apnea, hypothermia, hypotonia, somnolence, instillation site pain, oral dryness, burning and stinging, blurring, foreign body sensation, fatigue/drowsiness, conjunctival follicles, ocular allergic reactions, ocular pruritus, corneal staining/erosion, photophobia, eyelid erythema, ocular ache/pain, ocular dryness, tearing, upper respiratory symptoms, eyelid edema, conjunctival edema, blepharitis, ocular irritation, gastrointestinal symptoms, asthenia, abnormal vision, and muscular pain, in particular when compared to administration of a second composition comprising an alpha-2-adrenergic receptor agonists (e.g. brimonidine). In addition, without wishing to be bound by theory, the reduced binding of the compound of Formula I to iris pigments can lead to an amount of the compound of Formula I having the increased duration time of the therapeutic benefit when compared to an equivalent amount of an alpha adrenergic receptor agonist such as brimonidine, especially when the individual has an iris which would be considered a dark iris.

The frequency of administration of the compounds and compositions described herein for the treatment of the ocular conditions (e.g., reduced redness and/or increased whiteness) may vary from individual to individual. For example, the compound can be administered to the individual as needed, once a day (e.g., once about every 24 hours), twice a day (e.g., once about every 12 hours), three times a day (e.g., once about every 8 hours), four times a day (e.g., once about every 6 hours), or with any other frequency as can be determined by a skilled person such as a physician. In some aspects, the compounds and compositions described herein are administered to the eye of an individual once per day to reduce redness of the eye and/or increase whiteness of the eye. In some aspects, the compounds and compositions described herein are administered to the eye of an individual twice per day to reduce redness of the eye and/or increase whiteness of the eye.

Also described herein are pharmaceutical compositions that are useful for inducing ocular vasoconstriction, reducing ocular hyperemia, reducing eye redness, and/or increasing eye whiteness.

In one embodiment, the pharmaceutical composition comprising a compound of Formula I:

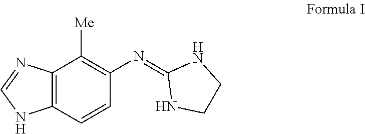 [SMILES: Cc1c(N=C2NCCN2)ccc2[nH]cnc12]

or a pharmaceutically acceptable salt thereof, a buffer, sodium chloride, and water. In another embodiment, the pharmaceutical composition consists essentially of a compound of Formula I, or a pharmaceutically acceptable salt thereof, a buffer, sodium chloride, and water.

The compound of Formula I can be synthesized by methods known to the skilled person (see, for example, U.S. Pat. Nos. 6,495,583 and 5,478,858).

In some embodiments, the buffer is a citrate buffer. In some embodiments, the buffer is a phosphate buffer. In some embodiments, the buffer is a citrate and phosphate buffer.

In some embodiments, the citrate buffer comprises citric acid and the phosphate buffer comprises a dibasic phosphate buffer such as dibasic sodium phosphate or dibasic potassium phosphate.

In some embodiments, the citric acid is present in the composition in an amount of between about 0.01% and about 1% (w/v), for example about 0.1% or about 0.09% (w/v).

In some embodiments, the citric acid is present in the composition in an amount of between about 0.089% and about 0.11% (w/v), between about 0.079% and about 0.13% (w/v), between about 0.071% and about 0.14% (w/v), between about 0.063% and about 0.16% (w/v), between about 0.056% and about 0.18% (w/v), between about 0.050% and about 0.20% (w/v), between about 0.045% and about 0.22% (w/v), between about 0.040% and about 0.25% (w/v), between about 0.035% and about 0.28% (w/v), between about 0.032% and about 0.32% (w/v), between about 0.028% and about 0.35% (w/v), between about 0.025% and about 0.40% (w/v), between about 0.022% and about 0.45% (w/v), between about 0.020% and about 0.50% (w/v), between about 0.018% and about 0.56% (w/v), between about 0.016% and about 0.63% (w/v), between about 0.014% and about 0.71% (w/v), between about 0.013% and about 0.79% (w/v), between about 0.011% and about 0.89% (w/v), between about 0.010% and about 1% (w/v), and ranges between any of these selected amounts of citric acid.

The source of the citric acid when making the pharmaceutical composition can be anhydrous citric acid, or it can be a hydrated form of citric acid (e.g. monohydrate) or any other form of citric acid suitable for making pharmaceutical compositions such as, for example, monosodium citrate (anhydrous), disodium citrate (sesquihydrate), trisodium citrate (anhydrous, dihydrate) and others identifiable to a skilled person upon a reading of the present disclosure such that the final amount of citric acid in the pharmaceutical composition is between about 0.01% and about 1% (w/v), for example about 0.1% or about 0.09% (w/v).

In some embodiments, the dibasic sodium phosphate is present in the composition in an amount of between about 0.01% and about 2% (w/v), for example about 0.5% or about 1% (w/v).

In some embodiments, the dibasic sodium phosphate is present in the composition in an amount of between about 0.79% and about 1.0% (w/v), between about 0.63% and about 1.1% (w/v), between about 0.50% and about 1.1% (w/v), between about 0.40% and about 1.1% (w/v), between about 0.32% and about 1.2% (w/v), between about 0.25% and about 1.2% (w/v), between about 0.20% and about 1.3% (w/v), between about 0.16% and about 1.3% (w/v), between about 0.13% and about 1.4% (w/v), between about 0.10% and about 1.4% (w/v), between about 0.079% and about 1.5% (w/v), between about 0.063% and about 1.5% (w/v), between about 0.050% and about 1.6% (w/v), between about 0.040% and about 1.6% (w/v), between about 0.032% and about 1.7% (w/v), between about 0.025% and about 1.7% (w/v), between about 0.020% and about 1.8% (w/v), between about 0.016% and about 1.9% (w/v), between about 0.013% and about 1.9% (w/v), between about 0.010% and about 2% (w/v), and ranges between any of these selected amounts of dibasic sodium phosphate.

In some embodiments, the dibasic sodium phosphate is present in the composition in an amount of between about 0.41% and about 0.54% (w/v), between about 0.34% and about 0.58% (w/v), between about 0.28% and about 0.62% (w/v), between about 0.23% and about 0.66% (w/v), between about 0.19% and about 0.71% (w/v), between about 0.15% and about 0.76% (w/v), between about 0.13% and about 0.81% (w/v), between about 0.10% and about 0.87% (w/v), between about 0.09% and about 0.93% (w/v), between about 0.071% and about 1.0% (w/v), between about 0.058% and about 1.1% (w/v), between about 0.048% and about 1.1% (w/v), between about 0.039% and about 1.2% (w/v), between about 0.032% and about 1.3% (w/v), between about 0.027% and about 1.4% (w/v), between about 0.022% and about 1.5% (w/v), between about 0.018% and about 1.6% (w/v), between about 0.015% and about 1.7% (w/v), between about 0.012% and about 1.9% (w/v), between about 0.010% and about 2% (w/v), and ranges between any of these selected amounts of dibasic sodium phosphate.

The source of the dibasic sodium phosphate when making the pharmaceutical composition can be anhydrous dibasic sodium phosphate, or it can be a hydrated form of dibasic sodium phosphate (e.g. heptahydrate) or any other form of sodium phosphate suitable for making pharmaceutical compositions such as, for example, sodium phosphate (anhydrous, hemihydrate, hexahydrate, octahydrate, dodecahydrate) and sodium phosphate monobasic (anhydrous, monohydrate, dihydrate) and disodium phosphate (anhydrous, dihydrate, octahydrate, dodecahydrate) and others identifiable to a skilled person upon a reading of the present disclosure such that the final amount of dibasic sodium phosphate in the pharmaceutical composition is between about 0.01% and about 1%, for example about 0.5% or about 1% (w/v).

In addition, in some embodiments, dibasic potassium phosphate can be used in place of the dibasic sodium phosphate. The amount of dibasic potassium phosphate can be similar to the amounts described herein for the dibasic sodium phosphate (adjusting for differences in molecular weight) but can ultimately be determined by a skilled person such that the pharmaceutical compositions comprising dibasic potassium phosphate would be pharmaceutically equivalent to those comprising dibasic sodium phosphate described herein.

In in some embodiments, the sodium chloride is present in an amount of between about 0.01% and about 1% (w/v), for example about 0.6% (w/v).

In some embodiments, the sodium chloride is present in an amount of between about 0.51% and about 0.65% (w/v), between about 0.42% and about 0.66% (w/v), between about 0.34% and about 0.68% (w/v), between about 0.28% and about 0.69% (w/v), between about 0.22% and about 0.71% (w/v), between about 0.18% and about 0.72% (w/v), between about 0.15% and about 0.74% (w/v), between about 0.12% and about 0.76% (w/v), between about 0.10% and about 0.78% (w/v), between about 0.079% and about 0.79% (w/v), between about 0.065% and about 0.81% (w/v), between about 0.052% and about 0.83% (w/v), between about 0.043% and about 0.85% (w/v), between about 0.035% and about 0.87% (w/v), between about 0.028% and about 0.89% (w/v), between about 0.023% and about 0.91% (w/v), between about 0.019% and about 0.93% (w/v), between about 0.015% and about 0.95% (w/v), between about 0.012% and about 0.98% (w/v), between about 0.010% and about 1% (w/v), and ranges between any of these selected amounts of sodium chloride.

In some embodiments, the amount of sodium chloride in the pharmaceutical composition can vary from the amount of sodium chloride initially added to make the composition if, for example, and acid such as hydrochloric acid or a base such as sodium hydroxide is used during the preparation of the pharmaceutical composition to adjust the pH. Accordingly, the amounts (including those in ranges) of sodium chloride described herein can be minimum amounts of sodium chloride present in the pharmaceutical compositions (e.g. at least 0.6% (w/v) sodium chloride).

In some embodiments, tonicity adjusters other than sodium chloride can be used. They include, but are not limited to, salts, particularly potassium chloride, mannitol, erythritol, carnitine, and glycerin, or any other suitable ophthalmically acceptable tonicity adjustor. The amounts of these tonicity adjusters can be determined by a skilled person such they would produce pharmaceutical compositions that are pharmaceutically equivalent to those comprising sodium chloride as described herein.

In some embodiments, the pharmaceutical composition is in the form of a solution suitable for ophthalmic application. The pharmaceutical composition can be prepared by combining the ingredients using methods identifiable to the skilled person for making pharmaceutical compositions suitable for ophthalmic application, such as good manufacturing procedures (GMP) for sterile liquid drug product.

In one embodiment, the pharmaceutical composition is prepared using a physiological saline solution as a major vehicle. In another embodiment, the major vehicle is water (e.g. purified water). The pH of such ophthalmic solutions should for example be maintained from about 4.5 to about 8.0 (e.g. a pH of about 7) with an appropriate buffer system, a neutral pH being preferred but not essential. Various buffers in addition to the citrate and phosphate buffers described herein and means for adjusting pH can be used so long as the resulting preparation is ophthalmically acceptable. Accordingly, addition buffers include, but are not limited to, acetate buffers and borate buffers. The amounts of these buffers can be determined by a skilled person such they would produce pharmaceutical compositions that are pharmaceutically equivalent to those comprising the citrate and/or phosphate buffers as described herein. In addition, acids or bases (e.g. an acid like hydrochloric acid or a base like sodium hydroxide) can be used to adjust the pH of these formulations as needed.

In some embodiments, the pH of the pharmaceutical composition is between about 6.9 and about 7.1, between about 6.8 and about 7.1, between about 6.6 and about 7.2, between about 6.5 and about 7.2, between about 6.4 and about 7.3, between about 6.3 and about 7.3, between about 6.1 and about 7.4, between about 6.0 and about 7.4, between about 5.9 and about 7.5, between about 5.8 and about 7.5, between about 5.6 and about 7.6, between about 5.5 and about 7.6, between about 5.4 and about 7.7, between about 5.3 and about 7.7, between about 5.1 and about 7.8, between about 5.0 and about 7.8, between about 4.9 and about 7.9, between about 4.8 and about 7.9, between about 4.6 and about 8.0, between about 4.5 and about 8.0, and ranges between those values.

In some embodiments, the pH of the pharmaceutical composition is between about 7.0 and about 7.1, between about 6.9 and about 7.1, between about 6.9 and about 7.2, between about 6.8 and about 7.2, between about 6.8 and about 7.3, between about 6.7 and about 7.3, between about 6.7 and about 7.4, between about 6.6 and about 7.4, between about 6.6 and about 7.5, between about 6.5 and about 7.5, between about 6.5 and about 7.6, between about 6.4 and about 7.6, between about 6.4 and about 7.7, between about 6.3 and about 7.7, between about 6.3 and about 7.8, between about 6.2 and about 7.8, between about 6.2 and about 7.9, between about 6.1 and about 7.9, between about 6.1 and about 8.0, between about 6.0 and about 8.0, and ranges between those values.

The pharmaceutical compositions can also contain conventional, pharmaceutically acceptable preservatives, stabilizers and surfactants. Exemplary preservatives that can be used in the pharmaceutical compositions include, but are not limited to, benzalkonium chloride, thimerosal, phenylmercuric acetate, phenylmercuric nitrate, chlorobutanol, methyl paraben, propyl paraben, phenylethyl alcohol, edetate disodium, ascorbic acid, polydronium chloride (e.g. ONAMER® M), stabilized oxychloro complex/stabilized chlorine dioxide (e.g. PURITE®), and other agents known to those skilled in the art. In ophthalmic products, typically such preservatives are employed at a level of from 0.004% to 0.02%. Stabilizers include, but are not limited to, polyvinyl alcohol, povidone, hydroxypropyl methyl cellulose, poloxamers, carboxymethyl cellulose, and hydroxyethyl cellulose cyclodextrin. In addition, the formulations can also be devoid of preservatives. Such formulations devoid of preservatives are said to be “preservative-free.”

The pharmaceutical compositions can also include a surfactant. Surfactants are useful to assist in dissolving an excipient or an active agent, dispersing a solid or liquid in a composition, enhancing wetting, modifying drop size, etc. Useful surfactants include, but are not limited to surfactants of the following classes: alcohols; amine oxides; block polymers; carboxylated alcohol or alkylphenol ethoxylates; carboxylic acids/fatty acids; ethoxylated alcohols; ethoxylated alkylphenols; ethoxylated aryl phenols; ethoxylated fatty acids; ethoxylated; fatty esters or oils (animal and/or vegetable); fatty esters; fatty acid methyl ester ethoxylates; glycerol esters; glycol esters; lanolin-based derivatives; lecithin and lecithin derivatives; lignin and lignin derivatives; methyl esters; monoglycerides and derivatives; polyethylene glycols; polymeric surfactants; propoxylated and ethoxylated fatty acids, alcohols, or alkyl phenols; protein-based surfactants; sarcosine derivatives; sorbitan derivatives; sucrose and glucose esters and derivatives.

An ophthalmically acceptable antioxidant can be included, and examples include sodium metabisulfite, sodium thiosulfate, acetylcysteine, butylated hydroxyanisole and butylated hydroxytoluene.

Other excipient components which can be included in the pharmaceutical compositions are chelating agents. An exemplary chelating agent is edetate disodium, although other chelating agents are known and suitable, alone or in combination with edetate disodium.

The pharmaceutical compositions can comprise the compound of Formula I in an amount between about 0.003% and about 1% (w/v). Unless otherwise indicated, the amounts of the compound of Formula I present in the compositions described herein are based on the freebase and non-solvated (non-hydrated) form compound (molecular weight 215.26), even if a salt and/or solvated (e.g. hydrate) form of the compound is used. Accordingly, all of the amounts of the compound of Formula I described herein will have corresponding amounts when the weight of the salt and/or solvate (e.g. hydrate) are taken into account, if such a form is used. For example, if the dihydrochloride monohydrate form of the compound (see Examples 8-11), which has a molecular weight of 306.19, is used, then a composition described herein as comprising the compound of Formula I in an amount of between about 0.0030% and about 1.0% (w/v) would be considered to comprise the dihydrochloride monohydrate form of the compound of Formula I in an amount of between 0.0043% and 1.4% due to the differences in molecular weight between the freebase and the dihydrochloride monohydrate form of the compound. Similarly, compositions described herein as comprising the compound of Formula I in an amount of about 0.010% (w/v), about 0.030% (w/v), 0.10% (w/v), 0.30% (w/v), or about 1.0% (w/v), would correspond to an amount of about 0.014% (w/v), about 0.043% (w/v), 0.14% (w/v), 0.43% (w/v), or about 1.4% (w/v), respectively, the dihydrochloride monohydrate form of the compound of Formula I. A skilled person will thus understand that all amounts of the compound of Formula I described herein will have equivalent amounts if salts and solvated (e.g. hydrated) forms of the compound are used, even if those amounts may be numerically the same as the amount of the freebase non-solvated form due to factors such as rounding and significant figures. For example, a 0.010% (w/v) amount of the compound of Formula I would correspond to a 0.014% (w/v) amount of the dihydrochloride monohydrate form of the compound to two significant figures, but said amount of the dihydrochloride monohydrate form can be rounded to 0.01% (w/v) if only one significant figure is considered.

The pharmaceutical compositions can also comprise the compound of Formula I in an amount between about 0.01% and about 1% (w/v), or between about 0.01% and about 0.2% (w/v), about 0.01% and about 0.3% (w/v), about 0.01% and about 0.4% (w/v), about 0.01% and about 0.5% (w/v), about 0.01% and about 0.5% (w/v), about 0.01% and about 0.6% (w/v), about 0.01% and about 0.7% (w/v), about 0.01% and about 0.8% (w/v), or about 0.01% and about 0.9% (w/v), and ranges in between any of these selected amounts of the compound of Formula I.

The pharmaceutical compositions can also comprise the compound of Formula I in an amount between about 0.01% and about 0.02% (w/v), between about 0.02% and about 0.03% (w/v), between about 0.03% and about 0.04% (w/v), between about 0.04% and about 0.05% (w/v), between about 0.05% and about 0.06% (w/v), between about 0.06% and about 0.06% (w/v), between about 0.06% and about 0.07% (w/v), between about 0.07% and about 0.08% (w/v), between about 0.08% and about 0.09% (w/v), between about 0.09% and about 0.10% (w/v), and ranges in between any of these selected amounts of the compound of Formula I.

The pharmaceutical compositions can also comprise the compound of Formula I in an amount between about 0.01% and about 0.06% (w/v), between about 0.06% and about 0.11% (w/v), between about 0.11% and about 0.16% (w/v), between about 0.16% and about 0.21% (w/v), between about 0.21% and about 0.26% (w/v), between about 0.26% and about 0.31% (w/v), between about 0.31% and about 0.36% (w/v), between about 0.36% and about 0.41% (w/v), between about 0.41% and about 0.46% (w/v), between about 0.46% and about 0.51% (w/v), between about 0.51% and about 0.55% (w/v), between about 0.55% and about 0.60% (w/v), between about 0.60% and about 0.65% (w/v), between about 0.65% and about 0.70% (w/v), between about 0.70% and about 0.75% (w/v), between about 0.75% and about 0.80% (w/v), between about 0.80% and about 0.85% (w/v), between about 0.85% and about 0.90% (w/v), between about 0.90% and about 0.95% (w/v), or between about 0.95% and about 1.00% (w/v), and ranges in between any of these selected amounts of the compound of Formula I.

In addition, the pharmaceutical compositions can comprise the compound of Formula I in an amount between about 0.001% and about 1% (w/v), or between about 0.001% and about 0.2% (w/v), about 0.001% and about 0.3% (w/v), about 0.001% and about 0.4% (w/v), about 0.001% and about 0.5% (w/v), about 0.001% and about 0.6% (w/v), about 0.001% and about 0.7% (w/v), about 0.001% and about 0.8% (w/v), or about 0.001% and about 0.9% (w/v), and ranges in between any of these selected amounts of the compound of Formula I.

The pharmaceutical compositions can also comprise the compound of Formula I in an amount between about 0.001% and about 0.01% (w/v), about 0.001% and about 0.02% (w/v), about 0.001% and about 0.03% (w/v), about 0.001% and about 0.04% (w/v), about 0.001% and about 0.05% (w/v), about 0.001% and about 0.06% (w/v), about 0.001% and about 0.07% (w/v), about 0.001% and about 0.08% (w/v), or about 0.001% and about 0.09% (w/v), about 0.001% and about 0.01%, and ranges in between any of these selected amounts of the compound of Formula I.

The pharmaceutical compositions can also comprise the compound of Formula I in an amount between about 0.001% and about 0.002% (w/v), between about 0.002% and about 0.003% (w/v), between about 0.003% and about 0.004% (w/v), between about 0.004% and about 0.005% (w/v), between about 0.005% and about 0.006% (w/v), between about 0.006% and about 0.006% (w/v), between about 0.006% and about 0.007% (w/v), between about 0.007% and about 0.008% (w/v), between about 0.008% and about 0.009% (w/v), between about 0.009% and about 0.010% (w/v), and ranges in between any of these selected amounts of the compound of Formula I.

In addition, the pharmaceutical compositions can comprise the compound of Formula I in an amount between about 0.003% and about 1% (w/v), or between about 0.003% and about 0.2% (w/v), about 0.003% and about 0.3% (w/v), about 0.003% and about 0.4% (w/v), about 0.003% and about 0.5% (w/v), about 0.003% and about 0.5% (w/v), about 0.003% and about 0.6% (w/v), about 0.003% and about 0.7% (w/v), about 0.003% and about 0.8% (w/v), or about 0.003% and about 0.9% (w/v), and ranges in between any of these selected amounts of the compound of Formula I.

The pharmaceutical compositions can also comprise the compound of Formula I in an amount between about 0.003% and about 0.01% (w/v), about 0.003% and about 0.02% (w/v), about 0.003% and about 0.03% (w/v), about 0.003% and about 0.04% (w/v), about 0.003% and about 0.05% (w/v), about 0.003% and about 0.06% (w/v), about 0.003% and about 0.07% (w/v), about 0.003% and about 0.08% (w/v), about 0.003% and about 0.09% (w/v), or about 0.003% and about 0.01%, and ranges in between any of these selected amounts of the compound of Formula I.

In addition, the pharmaceutical compositions can also comprise the compound of Formula I in an amount between about 0.1% and about 0.2% (w/v), about 0.2% and about 0.3% (w/v), about 0.3% and about 0.4% (w/v), about 0.4% and about 0.5% (w/v), about 0.5% and about 0.6% (w/v), about 0.6% and about 0.7% (w/v), about 0.7% and about 0.8% (w/v), about 0.8% and about 0.9% (w/v), or about 0.9% and about 1% (w/v), and ranges in between any of these selected amounts of the compound of Formula I. Additional amounts of the compound of Formula I for the compositions described herein would be identifiable to a skilled person upon a reading of the present disclosure.

In addition, the pharmaceutical compositions can also comprise the compound of Formula I in an amount between about 0.01% and about 0.02% (w/v), about 0.02% and about 0.03% (w/v), about 0.03% and about 0.04% (w/v), about 0.04% and about 0.05% (w/v), about 0.05% and about 0.06% (w/v), about 0.06% and about 0.07% (w/v), about 0.07% and about 0.08% (w/v), about 0.08% and about 0.09% (w/v), or about 0.09% and about 0.1% (w/v), and ranges in between any of these selected amounts of the compound of Formula I. Additional amounts of the compound of Formula I for the compositions described herein would be identifiable to a skilled person upon a reading of the present disclosure.

In addition, the pharmaceutical compositions can comprise the compound of Formula I in an amount of about 0.01% (w/v), about 0.03% (w/v), about 0.1% (w/v), or about 0.3% (w/v), and other amounts other than these selected amounts of the compound of Formula I. Additional amounts of the compound of Formula I for the compositions described herein would be identifiable to a skilled person upon a reading of the present disclosure.

Other amounts of the compound of Formula I or a pharmaceutically acceptable salt thereof for the compositions described herein would be identifiable to a skilled person upon reading the present disclosure.

In some embodiments, when the compound of Formula I is part of a pharmaceutical composition, the compound of Formula I is the only active ingredient which has therapeutic activity to induce ocular vasoconstriction, reduce ocular hyperemia, reduce redness of the eye, and/or to increase whiteness of the eye (and/or has therapeutic activity such that would be of use for the treatment or control of ocular conditions such as presbyopia, poor night vision, visual glare, visual starbursts, visual halos, and some forms of myopia (e.g. night myopia)). The term “active ingredient” as used herein refers to a component of a pharmaceutical composition which is responsible for the therapeutic effect of composition, whereas the other components of the composition (e.g. excipients, carriers, and diluents) are not responsible for the therapeutic effect of composition, even if they have other functions in the composition which are necessary or desired as part of the formulation (such as lubrication, flavoring, pH control, emulsification, stabilization, preservation, and other functions other than the therapeutic effect of composition as described herein). In particular, in some embodiments, pharmaceutical compositions described herein in which the compound of Formula I is the only active ingredient which has therapeutic activity are compositions in which there are no other components which would be considered to have therapeutic activity for inducing ocular vasoconstriction, reducing ocular hyperemia, reducing eye redness, and/or increasing eye whiteness (and/or have therapeutic activity such that would be of use for the treatment or control of ocular conditions such as presbyopia, poor night vision, visual glare, visual starbursts, visual halos, and some forms of myopia (e.g. night myopia)).

The ophthalmic formulation, in another embodiment, can be packaged in a form suitable for metered application, such as in a container equipped with a dropper, to facilitate application to the eye. Containers suitable for drop wise application are usually made of suitable inert, non-toxic plastic material, and generally contain from about 0.5 to about 15 ml solution. One package can contain one or more unit doses. Preservative-free solutions are often formulated in non-resealable containers containing up to about ten, such as up to about five units doses, where a typical unit dose is from one to about 8 drops, such as from one to about 3 drops. The volume of one drop usually is about 20-35 μL. In some embodiments, the containers can be multidose preservative-free (MDPF) containers (see, e.g. Chapter 20 of Ong, S. et al. Drug Development-A Case Study Based Insight into Modern Strategies 2011).

In addition, in some embodiments, various ocular delivery methods for administration to the eye are also contemplated for the compositions and/or compounds (e.g. compound of Formula I) described herein. For example, ocular administration methods can include, for example, topical administration, intravitreal administration, intracameral administration, and subconjunctival administration, and other ocular administration methods identifiable to a skilled person. In addition, additional administration methods such as using ocular drug delivery systems (e.g. ocular implants, intracameral implants, intravitreal implants, subconjunctival implants, sub-Tenon's implants, punctum plugs, canicular eluting implants, contact lenses loaded with the compound of Formula I, and ocular rings) are also envisioned for delivering the compounds and/or compositions described herein (for example, for sustained release over periods of days, weeks, or other periods recommended by a physician), as are injectable sustained-release formulations resulting in a depot, such as the compound of Formula I in a PLGA-based microsphere, which can also be used in any of the intraocular compartments such as the subconjunctival, sub-Tenon's, intracameral, and intravitreal spaces (see, e.g., Kuno Polymers 2011, 3, 193-221; U.S. Pat. Nos. 9,289,413 and 9,504,653; US Publication Nos. 2011/0182966, 2016/0022695, and 2016/0296532; and Chee, Journal of Ocular Pharmacology and Therapeutics 2012, 28 (4), 340-349, and Tejpal et al., J Drug Deliv. Therap. 2013, 3, 114-123). In some aspects, the compounds and compositions described herein are administered topically. In some aspects, the compounds and compositions described herein are administered topically as a solution.

Also contemplated is a kit comprised of a pharmaceutical composition comprising the compound of Formula I and instructions for administering the preparation to the eye. The pharmaceutical composition is, in one embodiment, provided or packaged in multidose form. In this embodiment, the pharmaceutical composition preferably comprises the compound of Formula I and a pharmaceutically acceptable excipient. Any of the excipients discussed herein are suitable for the ocular preparation. In one embodiment, the preparation comprises a preservative that prevents microbial contamination during use (i.e., repeated use).

The instructions for administration typically provide dosing instructions. In various embodiments, the instructions can be to administer the preparation once per day, twice per day or three times per day. In some embodiments where the preparation is a liquid preparation, the administration can be to place one drop, two drops, three drops, or more in the eye or in both eyes (e.g., if one eye is affected by the ocular condition, both eyes can be treated, or if both eyes are affected by the condition) once per day, twice per day, three times per day, or more.

In addition, the compounds and compositions (including delivery formulations and modes and kits) described herein for inducing ocular vasoconstriction, reducing ocular hyperemia, reducing eye redness, and/or increasing eye whiteness can also be of use in treating or controlling ocular conditions such as presbyopia, poor night vision, visual glare, visual starbursts, visual halos, and some forms of myopia (e.g. night myopia). The compositions can thus both treat ocular conditions such as presbyopia, poor night vision, visual glare, visual starbursts, visual halos, and some forms of myopia (e.g. night myopia) and also be of use for inducing ocular vasoconstriction, reducing ocular hyperemia, reducing eye redness, and/or increasing eye whiteness, for example, in individual who suffer from ocular conditions such as presbyopia, poor night vision, visual glare, visual starbursts, visual halos, and some forms of myopia (e.g. night myopia) and are also in need of (or desirous of) inducing ocular vasoconstriction, reducing ocular hyperemia, reducing eye redness, and/or increasing eye whiteness (e.g. an individual who both has presbyopia and wished to reduce eye redness/increase eye whiteness).

Accordingly, also described herein are pharmaceutical compositions and methods of improving vision in individuals in need thereof using the compositions, as well as methods of treating ocular conditions such as presbyopia, poor night vision, visual glare, visual starbursts, visual halos, and some forms of myopia (e.g. night myopia) in individuals in need thereof using the compositions described herein that are also of use for inducing ocular vasoconstriction, reducing ocular hyperemia, reducing eye redness, and/or increasing eye whiteness. Vision or visual improvement, including but not limited to near, intermediate, and/or distance visual acuity, can for example be reflected in the increase of number of letters correctly read at any time point post dosing, the increase in the average letter change, or 2-line or 3-line (at least) improvement, all from baseline (i.e., from pre-treatment) at different levels of illumination (for example, less than 200 cd/m², less than 150 cd/m², less than 100 cd/m², less than 50 cd/m², less than 10 cd/m², less than 5 cd/m², less than 2 cd/m², and ranges in between these luminance levels). Night vision improvement can be reflected in visual improvement for patients in dim or dark lighting (e.g., under mesopic or scotopic conditions). Day vision improvement can be reflected in visual improvement for patients in bright lighting as found during daylight hours or in sunshine (e.g., under photopic conditions). Vision improvement using the embodiments described herein can also be achieved in combination with or when using other visual aids and devices (especially those used for treating presbyopia), including but not limited to reading glasses, lens modifying medications, and surgical presbyopic options including intraocular lenses (IOLs).

In some embodiments, the ocular conditions are conditions which can be treated by constricting the size of the pupil. Without wishing to be bound by theory, the inventors believe that by constricting the pupil the “pinhole effect” is achieved which can have therapeutic effects such as improving depth of focus, visual acuity, and other effects of use in treating the ophthalmic conditions such as those described herein. In the pinhole effect, decreasing the pupil diameter increases the depth of focus and decreases the light scattering by blocking some peripheral light rays from entering the eye, thereby preventing unfocused light rays in the periphery from reaching the retina. These actions can help, for example, improve the quality of reading vision in presbyopes and night driving vision for commuters. Thus, conditions treatable by the methods described herein using the compositions described herein can include, for example, presbyopia, poor night vision, visual glare, visual starbursts, visual halos, and some forms of myopia (e.g. night myopia).

In view of the forgoing, also described herein are methods of treating ocular conditions such as presbyopia, poor night vision, visual glare, visual starbursts, visual halos, and some forms of myopia (e.g. night myopia) in an individual in need thereof using the pharmaceutical compositions described herein.

In one embodiment, the method comprises administering to the individual one of the pharmaceutical compositions described herein.

In another embodiment, the ocular condition being treated is selected from the group consisting of presbyopia, poor night vision, visual glare, visual starbursts, visual halos, and some forms of myopia (e.g. night myopia). Thus, described herein is a method of reducing pupil size for the treatment of an ocular condition in an individual in need of such treatment, the method comprising administering to the individual a therapeutically effective amount of a compound of Formula I, or a pharmaceutically acceptable salt thereof, wherein the ocular condition is selected from one or more of the group consisting of presbyopia, poor night vision, visual glare, visual starbursts, visual halos, and some forms of myopia (e.g. night myopia).

In some embodiments, the ocular condition is presbyopia. In other embodiments, the ocular condition is poor night vision. In other embodiments, the ocular condition is visual glare, visual starbursts, visual halos. In other embodiments, the ocular condition is a form of myopia (e.g. night myopia).

In addition, because the pharmaceutical compositions described herein are useful for constricting the pupil, they are of use in methods of treating ocular condition such as, for example, presbyopia, poor night vision, visual glare, visual starbursts, visual halos, and some forms of myopia (e.g. night myopia).

Accordingly, described herein are methods of treating ocular condition in an individual in need thereof comprising administering to the individual one of the pharmaceutical compositions described herein. In some embodiments, the ocular condition is selected from one or more of the group consisting of presbyopia, poor night vision, visual glare, visual starbursts, visual halos, and some forms of myopia (e.g. night myopia).

In some embodiments of the methods described herein, the pharmaceutical compositions can be administered directly to one or both of the eyes of the individual. In some embodiments, the pharmaceutical compositions can be administered to both eyes. In other embodiments, the pharmaceutical compositions can be administered to only one of the eyes.

In some embodiments of the methods described herein where the pharmaceutical compositions are administered directly to one or both eyes of the individual, the administration can be done topically to the eye or eyes.

As indicated above, the inventors have surprisingly found the compound of Formula I has greater in vivo activity than would have been predicted based in the in vitro activity of the compound of Formula I when compared to similar alpha-2-adrenergic receptor agonists, which can result in a greater duration of therapeutic activity (e.g. constricting the pupil, which is of use in methods of treating ocular condition such as, for example, presbyopia, poor night vision, visual glare, visual starbursts, visual halos, and some forms of myopia (e.g. night myopia)) of the compound of Formula I when compared to other alpha-2-adrenergic receptor agonists. Thus, in some embodiments, the amount of the compound of Formula I in the pharmaceutical compositions is an amount which, when administered to the individual, results in an increased efficacy and/or duration of effect when compared to other alpha-2-adrenergic receptor agonists (for example, brimonidine).

In particular, one effect of interest can be a reduction in pupil size (pupil constriction) when the pharmaceutical compositions are administered to an individual. Thus, in some embodiments, a particular amount of the compound of Formula I in the pharmaceutical compositions, when administered to an individual, can cause an amount of reduction in pupil size such that the pupil is constricted to a size of 3 mm or less, and in particular to a size of between 2 and 3 mm, from a natural baseline size which is larger than 3 mm. As would be apparent to a person skilled in the art, the natural baseline size of the pupil can depend on the particular lighting conditions/luminance levels (for example, less than 200 cd/m², less than 150 cd/m², less than 100 cd/m², less than 50 cd/m², less than 10 cd/m², less than 5 cd/m², less than 2 cd/m², and ranges in between these luminance levels) and age of the patient. Thus, baseline pupil sizes can range from about 6 to about 7 mm in low light to about 3 to about 4 mm in bright light, and in some embodiments, the therapeutically effective amount of the compound of Formula I can be an amount that reduces the pupil size from these baseline sizes to a size of 3 mm or less, and in particular to a size of between 2 and 3 mm. In some embodiments, these reductions in pupil size from baseline sizes can be achieved when the individual is exposed to luminance levels of, for example, less than 200 cd/m², less than 150 cd/m², less than 100 cd/m², less than 50 cd/m², less than 10 cd/m², less than 5 cd/m², less than 2 cd/m², and ranges in between these luminance levels.

A reduction of pupil size to a size of 3 mm or less, and in particular to a size of between 2 to 3 mm, can, for example, improve the at near reading ability of presbyopes, in particular at lower light conditions (see, e.g. Xu et al. “The effect of light level and small pupils on presbyopic reading performance.” Investigative ophthalmology & visual science 57, no. 13 (2016): 5656-5664.) However, brimonidine decreases pupil size to average 3.4 mm in presbyopic patients at different lighting conditions (see e.g. McDonald II et al. “Effect of brimonidine tartrate ophthalmic solution 0.2% on pupil size in normal eyes under different luminance conditions.” Journal of Cataract & Refractive Surgery 27, no. 4 (2001): 560-564), and thus is not ideal to improve depth of focus and improve reading acuity. The compound of Formula I has both a greater peak drop and longer duration of a pupil size being between 2 mm and 3 mm for a period of time between at least about 1 hour to at least about 9 hours while such durations of pupil constriction to 2-3 mm range are not seen when another alpha-2-adrenergic receptor agonist such as brimonidine is administered.

Thus, in some embodiments, a particular amount of the compound of Formula I in the pharmaceutical compositions, when administered to an individual, can have a duration of reduction in pupil size where the pupil is constricted to a size of 3 mm or less, and in particular to a size of between 2 and 3 mm for at least 1 hour, for at least 2 hours, for at least 4 hours, for at least 6 hours, or for at least 9 hours, for at least 10 hours, for at least 12 hours, and for ranges in between those times. In some embodiments, these pupil size reductions can be achieved when the individual is exposed to luminance levels of, for example, less than 200 cd/m², less than 150 cd/m², less than 100 cd/m², less than 50 cd/m², less than 10 cd/m², less than 5 cd/m², less than 2 cd/m², and ranges in between these luminance levels.

In other embodiments, a particular amount of the compound of Formula I in the pharmaceutical compositions, when administered to an individual, can have a duration of reduction in pupil size where the pupil is constricted to a size of about 2.0 mm for at least 1 hour, for at least 2 hours, for at least 4 hours, for at least 6 hours, for at least 9 hours, for at least 10 hours, or for at least 12 hours, and for ranges in between those times. In other embodiments, a particular amount of the compound of Formula I pharmaceutical compositions, when administered to an individual, can have a duration of pupil constriction where the pupil is constricted to a size of about 2.5 mm for at least 1 hour, for at least 2 hours, for at least 4 hours, for at least 6 hours, for at least 9 hours, for at least 10 hours, or for at least 12 hours and for ranges in between those times.

As indicated above, the inventors have also surprisingly found that, unlike the marketed alpha-2-adrenergic receptor agonist brimonidine (which has elevated binding to iris melanin pigments), the compound of Formula I does not exhibit much binding to iris melanin pigments. Thus, the pharmaceutical compositions can be administered with more consistent dosing between individuals having different eye colors/iris pigmentation (e.g. in individuals suffering from ocular condition such as, for example, presbyopia, poor night vision, visual glare, visual starbursts, visual halos, and some forms of myopia (e.g. night myopia)).

Thus, in some embodiments, the amount of the compound of Formula I in the pharmaceutical compositions is an amount which, when administered to the individual, results in reduced amount of binding to the iris pigment of the individual when compared to the administration of about the same amount of another alpha-2-adrenergic receptor agonist (for example, brimonidine). For example, in some embodiments, a particular amount of the compound of Formula I in the pharmaceutical compositions, when administered to an individual, can result in binding to the iris pigment that is about 8 to about 10 times less than the binding to the iris pigment when about the same amount of brimonidine is administered to the individual, especially when the individual has an iris which would be considered a dark iris (see, e.g. Franssen, L.; Coppens, J. E.; van den Berg, T. J., Grading of iris color with an extended photographic reference set. Journal of optometry 2008, 1 (1), 36-40).

Furthermore, this reduced amount of binding to iris pigments can result in reduced amount of compound of Formula I needed to achieve a particular therapeutic effect than would be needed if brimonidine were used, especially when the individual has an iris which would be considered a dark iris. Thus, in some embodiments, the amount of compound of Formula I needed would be about 30 to about 100 times less than the amount of brimonidine needed to achieve similar therapeutic effects as brimonidine (e.g. pupil constriction). In some embodiments, the amount of compound of Formula I needed would be about 30 times, about 40 times, about 50 times, about 60 times, about 70 times, about 80 times, about 90 times, or about 100 times less than the amount of brimonidine needed to achieve similar therapeutic effects as brimonidine (e.g. pupil constriction).

In addition, due to the reduced amount of the compound of Formula I needed, it is anticipated that the lower potential compound of Formula I needed would result in reduced incidence of side effects/adverse events normally associated with alpha-2-adrenergic receptor agonists (e.g. sedation). Accordingly, in some embodiments, pharmaceutical compositions described herein reduce the incidence of at least one adverse event selected from tachyphylaxis, rebound congestion, ocular hyperemia, eye irritation, increased intraocular pressure, eye pain, dizziness, fatigue, headache, hypotension, nasopharyngitis, sinusitis, bradycardia, iritis, miosis, skin reactions, erythema, eyelid pruritus, rash, vasodilation, tachycardia, apnea, hypothermia, hypotonia, somnolence, instillation site pain, oral dryness, burning and stinging, blurring, foreign body sensation, fatigue/drowsiness, conjunctival follicles, ocular allergic reactions, ocular pruritus, corneal staining/erosion, photophobia, eyelid erythema, ocular ache/pain, ocular dryness, tearing, upper respiratory symptoms, eyelid edema, conjunctival edema, blepharitis, ocular irritation, gastrointestinal symptoms, asthenia, abnormal vision, and muscular pain, in particular when compared to administration of a second composition comprising an alpha-2-adrenergic receptor agonists (e.g. brimonidine).

In addition, without wishing to be bound by theory, the reduced binding of the compound of Formula I to iris pigments can lead to an amount of the compound of Formula I having the increased duration time of the therapeutic benefit when compared to an equivalent amount of an alpha adrenergic receptor agonist such as brimonidine, especially when the individual has an iris which would be considered a dark iris.

In some embodiments of the methods described herein, the ocular condition being treated is presbyopia. Presbyopia is an age-related condition that affects nearly 1.7 billion people. In presbyopes, the ability of the eye to focus on near objects (accommodation) decreases with age and is believed to be caused by hardening of the lens of the eye on an individual as they age.

The extent and/or success of the treatment of presbyopia in an individual in need thereof can be determined by methods known to those skilled in the art (e.g. physicians and other medical workers). For example, an improvement in the uncorrected near visual acuity, intermediate visual acuity, and/or distance visual acuity when the pharmaceutical compositions are administered relative to the visual acuity when the pharmaceutical compositions are not administered. The improvement can be quantitatively measured by measuring the improvement in the number of lines correctly read by the patient on eye charts identifiable to those skilled in the art. For example, an individual can correctly read one or more (for example, two, three, or four) lines when the pharmaceutical compositions are administered to the individual than the number of lines the individual can correctly read prior to the administration of the pharmaceutical compositions. The improvement can be measured in one or both eyes, and under normal or low light conditions (for example, less than 200 cd/m², less than 150 cd/m², less than 100 cd/m², less than 50 cd/m², less than 10 cd/m², less than 5 cd/m², less than 2 cd/m², and ranges in between these luminance levels). In addition, non-quantitative (i.e. qualitative) measurements of the extend and/or success of the treatments can be measured, such as the individual's self-reporting of the improvement of the individual's vision after administration of the pharmaceutical compositions. For example, an individual might report improved reading ability and/or the lack of need for reading glasses after the administration of the pharmaceutical compositions. Additionally, an individual might also report reduced headaches and eye strain (which is normally present in the individual when the presbyopia is not being treated by other means such as reading glasses) when the individual is administered the pharmaceutical compositions.

Another measurement of the extent and/or success of the treatment of presbyopia in an individual in need thereof can be the measurement of the improvement in depth of focus (the distance, which can be measured in diopters or other units identifiable to a skilled person, that a viewed object can be moved away from and towards the individual before focus is lost) in an individual when the pharmaceutical compositions are administered to the individual relative to the depth of focus in the individual prior to the administration of the pharmaceutical compositions. The depth of focus can be measured and determined by methods identifiable to those skilled in the art, such as, for example, wavefront aberrometry and other methods identifiable to a skilled person.

Another measurement of the extent and/or success of the treatment of presbyopia in an individual in need thereof can be the measurement of pupil diameter and appearance in an individual when the pharmaceutical compositions are administered to the individual relative to the pupil diameter and appearance in the individual prior to the administration of the pharmaceutical compositions. The measurement of the pupil diameter and appearance can be measured by methods identifiable to a skilled person (e.g. using a wavefront aberrometer) under various lighting conditions identifiable to a skilled person so as to reflect nighttime outdoor and traffic lighting scenarios.

Another measurement of the extent and/or success of the treatment of presbyopia in an individual in need thereof can be the measurement of the change in visual field of an individual when the pharmaceutical compositions are administered to the individual relative to the visual field of the individual prior to the administration of the pharmaceutical compositions. The determination of an individual's visual field can be done by methods identifiable to a skilled person. For example, the individual can cover one eye while fixating on the eye of an examiner with the uncovered eye. The individual can then be asked to indicate the number of fingers briefly flashed by the examiner in each of the four quadrants (left, right, up, and down).

In some embodiments of the methods described herein, the ocular condition being treated is poor night vision. Many individuals suffer from poor night vision which is a condition in which an individual has impaired vision under low light conditions such as those occurring at night. The causes of the poor night vision can include corneal or lenticular aberrations which can be natural, but they can also result from ocular interventions such as laser surgery (e.g. LASIK). Without wishing to be bound by theory, the inventors believe that poor night vision can result when the pupil dilates under low light conditions which, for example if there are corneal or lenticular aberrations, can lead to some light rays not focusing on the pupil and that therefore improvement in night vision can be achieved if the pupil is constricted (e.g. by administering the pharmaceutical compositions to an individual with poor night vision).

The extent and/or success of the treatment of poor night vision in an individual in need thereof can be determined by methods known to those skilled in the art (e.g. physicians and other medical workers). For example, one measurement of the extent and/or success of the treatment of poor night vision in an individual can be an improvement in mesopic contrast sensitivity (with or without glare) as measured by systems identifiable to a skilled person (such as the Holladay Automated Contrast Sensitivity System, or HACSS™) when the pharmaceutical compositions are administered relative to the mesopic contrast sensitivity when the pharmaceutical compositions are not administered.

Another measurement of the extent and/or success of the treatment can be, for example, an improvement in the uncorrected near distance visual acuity, intermediate distance acuity, and/or distance acuity (all of which can be low contrast acuity or high contrast acuity; see for example Edwards, J. D.; Burka, J. M.; Bower, K. S.; Stutzman, R. D.; Sediq, D. A.; Rabin, J. C., Effect of brimonidine tartrate 0.15% on night-vision difficulty and contrast testing after refractive surgery. Journal of Cataract & Refractive Surgery 2008, 34 (9), 1538-1541) under low light conditions when the compound of Formula I is administered relative to the visual acuity when the compound is not administered. The improvement can be quantitatively measured by measuring the improvement in the number of lines correctly read by the patient under low light conditions on eye charts identifiable to those skilled in the art. For example, an individual can correctly read one or more (for example, two, three, or four) lines under low light conditions when the pharmaceutical compositions are administered to the individual than the number of lines the individual can correctly read prior to the administration of the pharmaceutical compositions. The improvement can be measured in one or both eyes.

In addition, non-quantitative (i.e. qualitative) measurements of the extend and/or success of the treatments can be measured, such as the individual's self-reporting of the improvement of the individual's vision under low light conditions after administration of the pharmaceutical compositions. For example, an individual might report improved night vision (e.g. while driving) and/or the lack of need for reading glasses under low light condition (e.g. at a restaurant with low lighting conditions) after the administration of the pharmaceutical compositions.

In some embodiments of the methods described herein, the ocular condition being treated is visual glare. Visual glare is a side effect of some ophthalmic surgeries such as laser surgery (e.g. LASIK) characterized by visual aberrations generally seen at night in which light enters the eye and interferes with vision. Without wishing to be bound by theory, the inventors believe that the visual aberrations seen in visual glare under low light conditions are caused and/or exacerbated by the additional light that enters the eye when the pupil dilates and can therefore be treated by constricting the pupil by administering the pharmaceutical compositions to a person experiencing visual glare.

In some embodiments of the methods described herein, the ocular condition being treated is visual starbursts. Visual starbursts are visual disturbances (which can be a side effect of some ophthalmic surgeries such as LASIK) in which light sources (such as street lamps and car headlights) appear to emit light in a starburst pattern emanating from the source of the light and which in some cases can obscure objects in close proximity to the light source, such as a pedestrian or cyclist that is near a headlight (see, e.g. the web page lasikcomplications.com/starbursting.htm). In other embodiments of the methods described herein, the ocular condition being treated is visual halos. Visual halos are another visual disturbance (which can be a side effect of some ophthalmic surgeries such as LASIK) that take the form of diffuse rings that can be seen around light sources, such street lamps, headlights, and illuminated reflective street signs (see, e.g. the web pages lasikcomplications.com/halos.htm and londonvisionclinic.com/post-lasik-patients-risk-of-halos-and-starbursts-around-bright-lights-at-night).

The extent and/or success of the treatment of visual glare, visual starbursts, and/or visual halos in an individual in need thereof can be determined by methods known to those skilled in the art (e.g. physicians and other medical workers). For example, the extent of the treatment can be determined by using tests known to a skilled person to assess the extent of visual glare, visual starbursts, and/or visual halos before and after having the compound of Formula I administered to them. For example, the severity of the visual glare, visual starbursts, and/or halos seen by an individual can be measured before administration of the pharmaceutical compositions and compared to the severity of the visual glare, visual starbursts, and/or halos seen by the individual after administration of the pharmaceutical compositions. The measurements can be qualitative (e.g. based on a questionnaire) or quantitative (e.g. by having the individual measure the size of a starburst and/or halo on a computerized optical system that can generate halos and starbursts) depending on the particular test used, which would be identifiable to a skilled person (see, e.g., Lee, J. H.; You, Y. S.; Choe, C. M.; Lee, E. S., Efficacy of brimonidine tartrate 0.2% ophthalmic solution in reducing halos after laser in situ keratomileusis. Journal of Cataract & Refractive Surgery 2008, 34 (6), 963-967 and Xu, R.; Kollbaum, P.; Thibos, L.; Lopez-Gil, N.; Bradley, A., Reducing starbursts in highly aberrated eyes with pupil miosis. Ophthalmic and Physiological Optics 2018, 38 (1), 26-36; and Hunkeler, J. D.; Coffman, T. M.; Paugh, J.; Lang, A.; Smith, P.; Tarantino, N., Characterization of visual phenomena with the Array multifocal intraocular lens. Journal of Cataract & Refractive Surgery 2002, 28 (7), 1195-1204). In addition, the extent of the treatment can also be self-reported by a patient after the patient has been administered the pharmaceutical compositions and has been able to drive at night while under its therapeutic effect.

In some embodiments of the methods described herein, the ocular condition being treated is a form of myopia (e.g. night myopia). For example, night myopia is a type of myopia (i.e. “nearsightedness”, of the inability to focus on distant objects) which tends to manifest itself at night and/or under low light conditions. Without wishing to be bound by theory, the inventors believe that night myopia can be caused by additional unfocused light rays entering the eye when the pupil is dilated under the lower light conditions and can thus be treated by reducing the size of the pupil by administering the pharmaceutical compositions to a person suffering from night myopia.

The extent and/or success of the treatment of night myopia in an individual in need thereof can be determined by methods known to those skilled in the art (e.g. physicians and other medical workers). For example, one measurement of the extent and/or success of the treatment can be, for example, an improvement in the intermediate distance acuity and/or distance acuity under low light conditions when the pharmaceutical compositions are administered relative to the visual acuity when the pharmaceutical compositions are not administered. The improvement can be quantitatively measured by measuring the improvement in the number of lines correctly read by the patient under low light conditions on eye charts identifiable to those skilled in the art. For example, an individual can correctly read one or more (for example, two, three, or four) lines under low light conditions when the pharmaceutical compositions are administered to the individual than the number of lines the individual can correctly read prior to the administration of the pharmaceutical compositions. The improvement can be measured in one or both eyes.

In addition, non-quantitative (i.e. qualitative) measurements of the extend and/or success of the treatments can be measured, such as the individual's self-reporting of the improvement of the individual's vision under low light conditions after administration of the pharmaceutical compositions. For example, an individual might report improved night distance vision (e.g. while driving) after the administration of the pharmaceutical compositions.

While the duration of the treatment of the ocular conditions (e.g. the amount of time vision is improved) may not be permanent, and may vary from individual to individual, the compound of Formula I can be administered in such a way so as to prolong the treatment of the presbyopia. For example, depending on the duration of the vision-improving effects of a particular dose of the compound of Formula I in the pharmaceutical compositions (which can be determined by a skilled person such as a physician), the compound can be administered once a day, twice a day, three times a day, four times a day, or with any other frequency as can be determined by a skilled person such as a physician.

In some embodiments, the pharmaceutical compositions described herein can show a similar or greater safety and/or efficacy profile when compared to the administration of a second composition comprising an ophthalmically active compound such as pilocarpine.

In particular, in some embodiments, the pharmaceutical compositions described herein can show a greater comparative safety profile by reducing the incidence of one or more adverse events selected from the group consisting of headache, brow ache, accommodative change, eye irritation, eye pain, blurred vision, visual impairment, ocular blurring, ocular discomfort, blurry vision, light sensitivity, stinging, and itching when compared to the administration of a second composition comprising an ophthalmically active compound such as pilocarpine.

In particular, in some embodiments, the pharmaceutical compositions described herein can show a greater comparative efficacy profile by demonstrating an improvement in one or more parameters described herein to determine the extent of treatment of the ocular conditions described herein, such as, for example, pupil size reduction, improvement in visual acuities (e.g. near, intermediate, and distance) at various illumination levels, improvement in depth of focus, improvement in visual filed, improvement of mesopic contrast sensitivity, as well as the clinical efficacy parameters described in Example 5 when compared to the administration of a second composition comprising an ophthalmically active compound such as pilocarpine. The improvement can be an improvement in magnitude (e.g. a greater amount of pupil size reduction or number of lines read in an acuity test) when compared to the administration of a second composition comprising an ophthalmically active compound such as pilocarpine. The improvement can also be an improvement in duration (e.g. a similar or greater amount of pupil size reduction for a longer period of time or similar or greater number of lines read in an acuity test at later timepoints after administration) when compared to the administration of a second composition comprising an ophthalmically active compound such as pilocarpine.

### EXAMPLES

The following examples are intended only to illustrate the methods of the present disclosure and should in no way be construed as limiting the methods of the present disclosure.

### Example 1

### In Vitro Activity of Alpha Adrenergic Receptor Agonists

An in vitro FLIPR (fluorometric image plate reader) assay was performed on several compounds, including the compound of Formula I (entry 1 in Table 3).

Specifically, four HEK293 stable cell lines were used in the FLIPR assay. The HEK293 cell line which stably expressed the bovine alpha adrenergic 1A receptor was used to characterize the alpha1 pharmacology. The alpha-2 adrenergic receptor family is a G coupled G-protein receptor. Therefore, in order to use these cell lines in the calcium based FLIPR assay, the chimeric G-protein Gqi5 was used to force the coupling of the human alpha-2A, alpha-2B, and alpha-2C receptors to the calcium pathway. Cells were plated, in triplicate, in poly-D-lysine coated 384-well plates at 25,000 cells per well and grown overnight in DMEM supplemented with 10% fetal bovine serum. For FLIPR evaluation, cells were washed twice with HBSS/HEPES buffer (1× Hanks Buffered Salt Solution, 20 mM HEPES, pH 7.4) prior to the addition of Fluo-4-AM (4 uM Fluo-4-AM, 0.04% pluronic acid in HBSS/HEPES buffer), a calcium-sensitive dye. Cells were loaded with dye for 40 minutes at 37° C., and then washed 4 times with HBSS/HEPES buffer to remove the excess dye. The test compounds were profiled at concentrations between 0.64 nM and 10,000 nM using a four-fold dilution factor. Norepinephrine was used as the standard full agonist for evaluating the alpha-1 receptor relative efficacy and brimonidine (Compound 4) was used as the standard full agonist for evaluating the alpha-2 receptor relative efficacy. Either norepinephrine or brimonidine was tested at concentrations between 0.064 nM and 1000 nM using a four-fold dilution factor.

The receptor activation was initiated by the addition of the appropriate dilutions of compounds and the transient calcium signal was captured. The peak height of the calcium curve was determined and utilized for calculation of EC₅₀ and relative efficacy values using Activity Base software. EC₅₀ was calculated using a 4 Parameter Logistic Equation: y=A+((B−A)/(1+((C/x)\hat{ }D))) where A and B represents the bottom and top plateau of the curve; C represents the EC₅₀ value; D represents the slope factor; and x and y represent the original x (drug concentration) and y (fluorescence signal, RFU) values.

TABLE 3

| | | bovine | human |
|---|---|---|---|
| | | alpha1A | alpha2A |
| Compound | Structure | EC₅₀ (nm) | EC₅₀ (nm) |
| 1 | 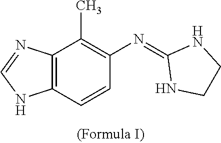 [SMILES: Cc1c(N=C2NCCN2)ccc2[nH]cnc12] | 286 | 0.7 |
| 2 | 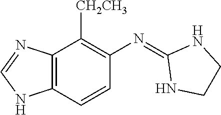 [SMILES: CCc1c(N=C2NCCN2)ccc2[nH]cnc12] | 1651 | 11 |
| 3 | 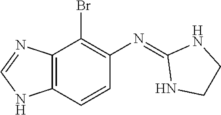 [SMILES: Brc1c(N=C2NCCN2)ccc2[nH]cnc12] | 1000 | 1.0 |
| 4 | 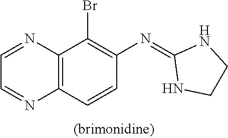 [SMILES: Brc1c(N=C2NCCN2)ccc2nccnc12] | 1205 | 0.8 |

Based on the in vitro pharmacology, it would have been expected that alpha-2-adrenergic receptor pan-agonists such as compounds 2 and 3 and the compound of Formula I (compound 1) would have similar efficacy in rabbit like brimonidine (compound 4).

### Example 2

### Melanin Binding

An assay was performed in which the melanin binding of the compound of Formula I was measured and compared to the melanin binding of additional compounds including brimonidine (the binding of which was previously determined by the inventors).

In particular, the compound of Formula I (Compound 1), Compound 2, and a positive control (chloroquine; Compound 5) were tested for binding to synthetic melanin. The testing concentrations ranged from 1.29 ng/mL to 12,500 ng/mL for the compound of Formula I (Compound 1) and Compound 2, and from 19.8 to 8000 ng/mL for chloroquine. Compound stock solutions were prepared in dimethyl sulfoxide with 0.5% or 0.6% (v/v) formic acid (Compound 1 and Compound 2, respectively) or in water (chloroquine) and then further diluted in PBS to the specified curve range and incubated at 37° C. for 1 hour with and without melanin. Aliquots of the PBS only curve were quenched at time zero to be used as stability controls and calibration standards. Following centrifugation, samples were analyzed by LC-MS/MS bioanalysis. Back-calculated concentrations using the assay PBS curve were used for binding and stability calculations. The results of the melanin binding assay and the comparison to previously determined melanin binding of brimonidine can be seen in Table 4.

TABLE 4

| | | Mean % bound across |
|---|---|---|
| Compound | Structure | all concentrations |
| 1 | 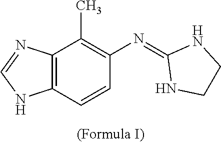 [SMILES: Cc1c(N=C2NCCN2)ccc2[nH]cnc12] | 8.6 |
| 2 | 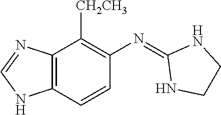 [SMILES: CCc1c(N=C2NCCN2)ccc2[nH]cnc12] | 9.1 |
| 4 | 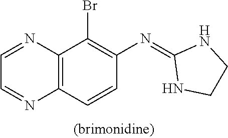 [SMILES: Brc1c(N=C2NCCN2)ccc2nccnc12] | 80 |
| 5 | Chloroquine | 94.9 |
| | (positive control) | |

As can be seen from Table 4, the compound of Formula I exhibits significantly and unexpectedly lower binding that other alpha-2-adrenergic receptor agonists including brimonidine. In particular, with mean binding percentages of around 10% or less, the compound of Formula I could be considered to have no significant melanin binding, in contrast to the much more significant binding seen with brimonidine.

### Example 3

### Reducing Eye Redness and/or Increasing Eye Whiteness in Rabbits

In this study, Applicant evaluated the compound of Formula I on ocular hyperemia (i.e., eye redness).

Study Design: The effects of topical ocular treatment of the compound of Formula I on ocular hyperemia were assessed in naïve rabbits and compared to LUMIFY® (0.025% brimonidine tartrate by Bausch & Lomb), which has been shown to enhance the cosmetic appearance of eyes via conjunctival blanching (McLaurin et al, Optom Vis Sci., 95(3):264-271 (2018)).

The modified Hackett-McDonald Scoring scale (Table 1) was used to assess hyperemia, discomfort, conjunctival swelling and discharge. Ocular imaging was conducted via a Haag-Streit B slit lamp. Effects of these compounds were first evaluated in naïve animals following baseline measurements and then assessed in a povidone iodine induced hyperemia model.

Animals: This study was conducted in female New Zealand White rabbits (Covance) weighing approximately 2-4 kg upon arrival.

Model Induction: Disinfection of the conjunctiva, lids, and lashes by 5-10% povidone iodine has been demonstrated to result in transient hyperemia (Ridder et al, Optom Vis Sci. 94(7):732-741 (2017)). This effect can be utilized as a transient hyperemia model to evaluate for potential blanching of blood vessels and rapid reduction of hyperemia. The ocular surface was anesthetized with drops of 0.5% proparacaine. The eye was then irrigated with 5% povidone iodine ophthalmic solution and left on the ocular surface for at least 2 minutes to ensure the solution completely covers the eye. The periocular region was also wiped with povidone solution. Following 2 minutes post-irrigation, the eye was rinsed with sterile saline until the rinsed solution runs clear. Hyperemia at baseline and post ocular topical treatment was assessed via the modified Hackett-McDonald Scoring Scale shown in Table 1 above.

Test Agents: Rabbits were treated acutely by ocular instillation of a single eye drop (˜35 μl) composed of either 0.003% of the compound of Formula I, 0.03% of the compound of Formula I, or LUMIFY© (0.025% brimonidine tartrate).

Ocular imaging: A Haag-Streit BP 900 slitlamp was used to evaluate the anterior segment of the eye (e.g., corneal integrity and cells in the aqueous humor). Pupils were dilated by applying drops of 1% cyclopentolate and 10% phenylephrine to the ocular surface. Ocular imaging was conducted in awake and/or anesthetized animals (7.5-10 mg/kg Ketamine/1-10 mg/kg Xylazine).

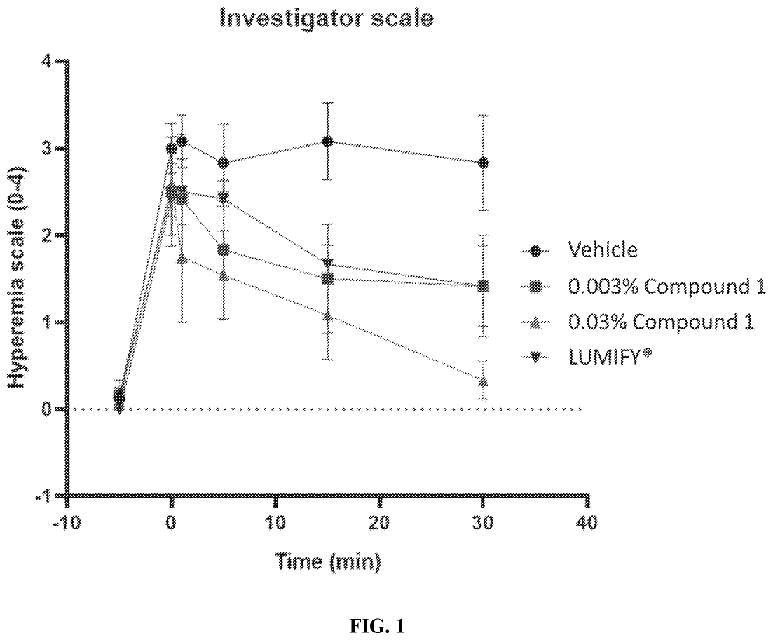

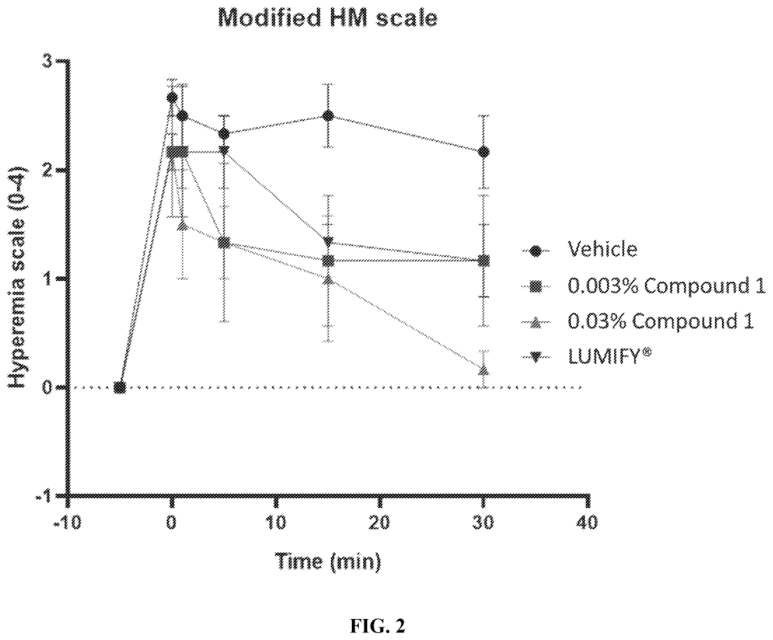

Results: As can be seen from FIG. 1 and FIG. 2, the results demonstrate that the 0.03% composition of compound of Formula I has significantly greater efficacy on ocular hyperemia than LUMIFY®. For example, the compound of Formula I provided a greater reduction in the Modified Hackett-McDonal Score (including hyperemia, discomfort, conjunctival swelling and discharge) than LUMIFY®.

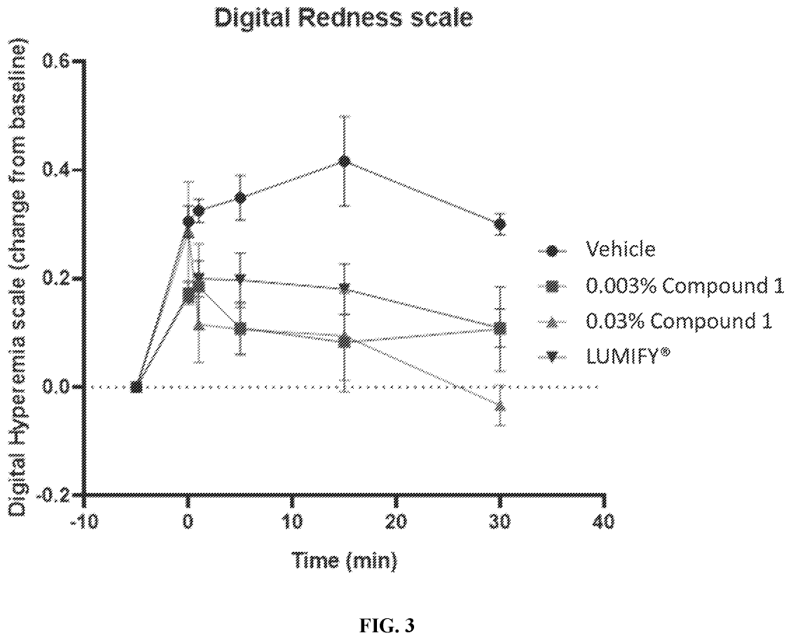

In addition, as can be seen from FIG. 3, the 0.03% composition of compound of Formula I also performed better than LUMIFY® with significant reduction in hyperemia as early as 1 min post treatment.

### Example 4

### Clinical Study of Reduction of Hyperemia in Healthy Individuals

In this study, 36 healthy participants (male and female; 40 to 65 years of age) are randomized to participate in single-ascending dose cohorts (designated Cohorts 1, 2, and 3 as shown in Table 9 below) to evaluate safety, tolerability, pharmacokinetics, and target engagement of the compound of Formula I. In each cohort of this study 12 participants are randomized in a 3:1 ratio to receive an ophthalmic solution containing the compound of Formula I (9 participants) or vehicle (3 participants). Participants that are randomized to receive the ophthalmic solution containing the compound of Formula I are administered a single drop of the ophthalmic solution containing the compound of Formula I at doses ranging from 0.01% (w/v) to 0.1% (w/v) (Formulations A-C; see Table 6 below), with the option of also using a 0.3% (w/v) dose (Formulation D; see Example 11), in the left eye, as indicated in Table 5 below (“SD” means single dose and “OS” means left eye).

TABLE 5

| Cohort 1 | Cohort 2 | Cohort 3 |
|---|---|---|
| Formulation A | Formulation B | Formulation C |
| (SD OS) | (SD OS) | (SD OS) |
| 9 Active; 3 Vehicle | 9 Active; 3 Vehicle | 9 Active; 3 Vehicle |

The three Formulations A to C, and optional Formulation D, are shown in Table 6 below.

TABLE 6

| | | | |
|---|---|---|---|
| Component | Concentration | Function | Grades |

Formulation A

| | | | | |
|---|---|---|---|---|
| Compound 1 | 0.01 | (% w/v)¹ | Active | — |
| Citric acid | 0.1 | (% w/v) | Buffering Agent | USP |
| (monohydrate) | | | | |
| Dibasic sodium | 1.0 | (% w/v) | Buffering Agent | USP |
| phosphate | | | | |
| (heptahydrate) | | | | |
| Sodium chloride | 0.6 | (% w/v) | Tonicity Adjust | USP |
| Hydrochloric | to pH | 7.0 | pH Adjust | NF/Ph Eur |
| acid and/or | | | | |
| sodium hydroxide | | | | |
| Purified water | to 100 | (% w/v) | QS Adjust | Ph Eur/USP |

Formulation B

| | | | | |
|---|---|---|---|---|
| Compound 1 | 0.03 | (% w/v)¹ | Active | — |
| Citric acid | 0.1 | (% w/v) | Buffering Agent | USP |
| (monohydrate) | | | | |
| Dibasic sodium | 1.0 | (% w/v) | Buffering Agent | USP |
| phosphate | | | | |
| (heptahydrate) | | | | |
| Sodium chloride | 0.6 | (% w/v) | Tonicity Adjust | USP |
| Hydrochloric | to pH | 7.0 | pH Adjust | NF/Ph Eur |
| acid and/or | | | | |
| sodium hydroxide | | | | |
| Purified water | to 100 | (% w/v) | QS Adjust | Ph Eur/USP |

Formulation C

| | | | | |
|---|---|---|---|---|
| Compound 1 | 0.1 | (% w/v)¹ | Active | — |
| Citric acid | 0.1 | (% w/v) | Buffering Agent | USP |
| (monohydrate) | | | | |
| Dibasic sodium | 1.0 | (% w/v) | Buffering Agent | USP |
| phosphate | | | | |
| (heptahydrate) | | | | |
| Sodium chloride | 0.6 | (% w/v) | Tonicity Adjust | USP |
| Hydrochloric | to pH | 7.0 | pH Adjust | NF/Ph Eur |
| acid and/or | | | | |
| sodium hydroxide | | | | |
| Purified water | to 100 | (% w/v) | QS Adjust | Ph Eur/USP |

Formulation D

| | | | | |
|---|---|---|---|---|
| Compound 1 | 0.3 | (% w/v)¹ | Active | — |
| Citric acid | 0.1 | (% w/v) | Buffering Agent | USP |
| (monohydrate) | | | | |
| Dibasic sodium | 1.0 | (% w/v) | Buffering Agent | USP |
| phosphate | | | | |
| (heptahydrate) | | | | |
| Sodium chloride | 0.6 | (% w/v) | Tonicity Adjust | USP |
| Hydrochloric | to pH | 7.0 | pH Adjust | NF/Ph Eur |
| acid and/or | | | | |
| sodium hydroxide | | | | |
| Purified water | to 100 | (% w/v) | QS Adjust | Ph Eur/USP |

Notes:
¹Compound was the dihydrochloride monohydrate form; final concentration based on free base anhydrous form.

The vehicle for the three cohorts is shown in Table 7 below.

TABLE 7

| Component | Concentration | | Function | Grades |
|---|---|---|---|---|
| Citric acid | 0.1 | (% w/v) | Buffering Agent | USP |
| (monohydrate) | | | | |
| Dibasic sodium | 1.0 | (% w/v) | Buffering Agent | USP |
| phosphate | | | | |
| (heptahydrate) | | | | |
| Sodium chloride | 0.6 | (% w/v) | Tonicity Adjust | USP |
| Hydrochloric | to pH | 7.0 | pH Adjust | NF/Ph Eur |
| acid and/or | | | | |
| sodium hydroxide | | | | |
| Purified water | to 100 | (% w/v) | QS Adjust | Ph Eur/USP |

Each participant is admitted to the study site on the evening before dosing (Day −1). Participants receive their single dose in the morning of the first day (Day 1) and, after a full day of assessments, stay overnight and continue further safety and pharmacokinetic assessments on the next day (end-of-study visit). After each cohort is completed, an independent data monitoring committee reviews the safety, pharmacokinetic, and target engagement data to determine if it is acceptable to proceed to the next planned dosing cohort. Once Cohort 2 of this study is completed, in addition to making a recommendation on whether to initiate the final cohort of this study (Cohort 3), the data monitoring committee also makes a recommendation on whether to start the next study (Cohort 4 in Example 4 below) simultaneously.

Safety and tolerability are evaluated by monitoring participants in the three cohorts of this study for exhibition/reporting of adverse events and monitoring characteristics/assessments such as vital signs, electrocardiogram measurements, clinical laboratory assessments, study intervention tolerability and drop comfort assessments, intraocular pressure measurements, slit-lamp biomicroscopy, and dilated funduscopic examination. Systemic and local pharmacokinetics of the compound of Formula I are evaluated by measuring plasma and tear concentrations of the compound of Formula I in the participants and also determining pharmacokinetic parameters such as area under the curve, clearance, half-life, maximum concentration, time to maximum concentration. Target engagement is measured by measuring the pupil size of the participants during the study. In particular, pupil diameter is measured with a pupilometer for near (40 cm) and distance (4 meter) targets in mesopic (e.g. 3.2 to 3.5 cd/m²; 10 to 11 lux at target) and photopic (e.g. ≥80 cd/m²; 251 lux at target) conditions. In addition, bulbar conjunctival hyperemia will be assessed in each eye will be graded separately by the investigator using sponsor-provided reference photographs, as well as by slit lamp examination (see Example 5 and Table 10 below). Consistent illumination will be attempted to be maintained be maintained throughout the study.

At the end of this study, in all three cohorts (or at least one cohort), the ophthalmic solutions of the compound of Formula I administered as single doses demonstrate acceptable safety and tolerability profiles in the participants. Additionally, in all three cohorts (or at least one cohort), the ophthalmic solutions of the compound of Formula I administered as single doses result in systemic exposures in the participants with sufficient safety margins. Additionally, in all three cohorts (or at least one cohort), the ophthalmic solutions of the compound of Formula I administered as single doses demonstrate a decrease in pupil diameter in the participants as compared with vehicle. In addition, the ophthalmic solutions of the compound of Formula I administered as single doses demonstrate a reduction bulbar conjunctival hyperemia and/or whitening of the eye.

### Example 5

### Clinical Study in Presbyopes (Including Reduction of Hyperemia

In this study, approximately 108 participants diagnosed with presbyopia (male and female; 40 to 65 years of age) are selected to participate. This study is further divided into Parts A and B, as can be seen from Table 8 below (“QD” means once daily, “OU” means both eyes, and “TBD” means to be determined based on the results of Part A; Formulations A-C refer to those in Example 4 and there is the option of using the Formulation of using Formulation D from Example 4).

TABLE 8
Part A

| Cohort 4 | Cohort 5 | Cohort 6 |
|---|---|---|
| Formulation A | Formulation B | Formulation C |
| (QD × 14 days OU) | (QD × 14 days OU) | (QD × 14 days OU) |
| 9 Active; 3 Vehicle | 9 Active; 3 Vehicle | 9 Active; 3 Vehicle |

Part B

| Cohort 7 | Cohort 8 |
|---|---|
| Compound of Formula I | Compound of Formula I |
| (dose strength TBD) | (dose strength TBD) in |
| versus pilocarpine 1.25% (w/v) | participants who wear contact lenses |
| (QD × 14 days OU) | (QD × 14 days OU) |
| 60 (30 per treatment arm) | 9 Active; 3 Vehicle |

The vehicle for the three cohorts is the same as the one in Example 4 above. The pilocarpine composition is shown in table 9 below.

TABLE 9

| Component | Concentration | | Function | Grades |
|---|---|---|---|---|
| Pilocarpine | 1.25 | (% w/v) | Active | Ph Eur/USP |
| hydrochloride | | | | |
| Benzalkonium | 0.0075 | (% w/v) | Preservative | NF/Ph Eur/JP |
| chloride | | | | |
| Boric acid | 1.0 | (% w/v) | Buffering Agent | NF/Ph Eur |
| Sodium citrate | 0.015 | (% w/v) | Buffering Agent | Ph Eur/USP |
| dihydrate | | | | |
| Sodium chloride | 0.08 | (% w/v) | Tonicity Adjust | Ph Eur/USP |
| Hydrochloric | to pH | 5.0 | pH Adjust | NF/Ph Eur |
| acid and/or | | | | |
| sodium hydroxide | | | | |
| Purified water/ | to 100 | (% w/v) | Diluent | Ph Eur/USP |
| water for | | | | |
| injection | | | | |

Cohorts 4, 5, and 6 (Part A) are multiple ascending dose cohorts in which participants are randomized to receive either the compound of Formula I at doses ranging from 0.01% to 0.1% or vehicle once daily in both eyes for 14 days. Each cohort in Part A consists of 9 participants who receive active intervention (the compound of Formula I) and 3 participants that receive vehicle in order to evaluate safety, tolerability, pharmacokinetics, and clinical efficacy of the compound of Formula L. As with Example 4, after each cohort is completed, the independent data monitoring committee reviews the safety, pharmacokinetic, and target engagement data to determine if it is acceptable to proceed to the next planned dosing cohort. The dose of the compound of Formula I in Part B (Cohorts 7 and 8) is selected by the data monitoring committee after Cohort 6 has completed.

Cohort 7 is a single-masked, active comparator cohort that includes approximately 60 masked participants with presbyopia randomized 1:1 to receive either pilocarpine 1.25% (w/v) ophthalmic solution or an ophthalmic solution of the compound of Formula I administered at the dose strength recommended by the data monitoring committee for 14 days. Safety, tolerability, and efficacy is assessed after topical ocular administration of the compound of Formula I compared with pilocarpine 1.25%. Common adverse events associated with pilocarpine (particularly when administered to the eye) include, for example, headache, brow ache, accommodative change, eye irritation, eye pain, blurred vision, visual impairment, ocular blurring, ocular discomfort, blurry vision, light sensitivity, stinging, and itching.

Cohort 8 is a cohort to evaluate safety, tolerability, pharmacokinetics, and efficacy of the compound of Formula I administered at the dose strength recommended by the data monitoring committee compared with vehicle for 14 days in participants with presbyopia who wear contact lenses (9 participants receive the compound of Formula I and 3 participants receive vehicle).

In all cohorts of this study (Cohorts 4 to 8), participants receive their first dose in clinic on Day 1, and stay in clinic for safety, pharmacokinetic, and efficacy evaluations for 10 hours post-dose. The participants then return for dosing and safety evaluations once daily from Day 2 to Day 7. They then receive multidose bottles for single unit of use for at-home dosing from Days 8 to 13 and return to the clinic on Day 14 for dosing as well as safety, tolerability, pharmacokinetic (Cohorts 4 to 6, and 8 only), and efficacy evaluations for 10 hours post-dose. On Day 30, participants return for a safety follow-up and close out visit.

In Parts A and B of this study, safety and tolerability are evaluated as in Example 4 (i.e. by monitoring participants in the three cohorts for exhibition/reporting of adverse events and monitoring the characteristics/assessments as are monitored in Example 4), with the additional characteristics/assessments of contrast sensitivity, photopic (e.g. ≥80 cd/m²; 251 lux at target) and mesopic (e.g. 3.2 to 3.5 cd/m²; 10 to 11 lux at target) high contrast corrected distance visual acuity (each eye and binocularly), manifest refraction (mesopic and photopic) also being measured, and also bulbar conjunctival hyperemia being assessed in each eye will be graded separately by the investigator using sponsor-provided reference photographs and by slit lamp biomicroscopic slit-lamp examination, which will also evaluate palpebral conjunctival hyperemia. For the slit lamp examination, the conjunctival hyperemia will be evaluated according to the scale described in table 10 below. Consistent illumination will be attempted to be maintained be maintained throughout the study. Systemic and local pharmacokinetics of the compound of Formula I are evaluated by measuring plasma and tear concentrations of the compound of Formula I in the participants and also determining pharmacokinetic parameters as is done in Example 4. Target engagement is measured by measuring the pupil size of the participants during the study as is done in Example 4.

TABLE 10

| | |
|---|---|
| Value | Description |

Bulbar Conjunctival Hyperemia

| | | |
|---|---|---|
| 0 | (None) | No hyperemia |
| +0.5 | (Trace) | Minimal (trace) flush, reddish color |
| +1 | (Mild) | Mild flush, reddish color |
| +2 | (Moderate) | Bright red color |
| +3 | (Severe) | Deep, bright diffuse redness |

Palpebral Conjunctival Hyperemia

| | | |
|---|---|---|
| 0 | (None) | No hyperemia |
| +0.5 | (Trace) | Minimal (trace) flush, reddish color |
| +1 | (Mild) | Mild flush, reddish color |
| +2 | (Moderate) | Bright red color |
| +3 | (Severe) | Deep, bright diffuse redness |

Furthermore, in Parts A and B of this study, clinical efficacy of the compound of Formula I (compared with vehicle in Part A and compared with 1.25% (w/v) pilocarpine in Part B) is primarily determined by measuring the proportion of participants showing a 3-line improvement in mesopic (e.g. 3.2 to 3.5 cd/m²; 10 to 11 lux at target), high contrast, binocular distance corrected near visual acuity at Day 14, 3 hours after dosing. Secondarily, clinical efficacy is also determined by measuring the proportion of participants showing a 3-line or 2-line improvement in mesopic (e.g. 3.2 to 3.5 cd/m²; 10 to 11 lux at target), high contrast, binocular distance corrected near (Parts A and B) and intermediate (Part B) visual acuity; the change from baseline mesopic (e.g. 3.2 to 3.5 cd/m²; 10 to 11 lux at target), high contrast, binocular distance-corrected near visual acuity letters; and the change from baseline in mesopic (e.g. 3.2 to 3.5 cd/m²; 10 to 11 lux at target) and photopic (e.g. ≥80 cd/m²; 251 lux at target) pupil diameter.

Additionally, clinical efficacy in Parts A and B of this study is also determined measurement of depth of focus (as measured with a phoropter) and by five patient reported outcome questionnaires/questions: 1) a Near Vision Presbyopia Task-based Questionnaire (NVPTQ), 2) an electronic Near Vision Presbyopia Task-based Questionnaire (e-NVPTQ), 3) a Presbyopia Impact and Coping Questionnaire (PICQ), 4) a single-item Patient Global Impression of Status (PGIS) question, and 5) a single-item Patient Global Impression of Change (PGIC) question.

In the NVPTQ, which is comprised of 12 questions on four reading tasks (specifically, reading a paragraph from a book, reading excerpts from an article in a newspaper, reading a portion of a nutrition label, and reading a section from a restaurant menu), the participants complete specific reading tasks under mesopic (e.g. 3.2 to 3.5 cd/m²; 10 to 11 lux at target) conditions without any near-vision correction. Participants then answer 3 questions for each task, rating their vision-related reading ability and satisfaction with their vision-related reading ability.

In the e-NVPTQ, which is comprised of 3 questions on an electronic reading task (specifically reading a text on iPhone), the participants complete the specific reading task under photopic (e.g. ≥80 cd/m²; 251 lux at target) conditions without any near-vision correction. Participants then answer 3 questions for the task, rating their vision-related reading ability and satisfaction with their vision-related reading ability.

In the PICQ, the participants answer 20 questions about the degree to which they were impacted by their difficulty seeing up close (e.g., found daily near vision tasks difficult, or experienced self-consciousness); or engaged in coping behaviors (e.g., changed the font size on electronic screens) during the previous 7 days.

In the single-question PGIS question, the participants answer a single-item question about their global impression of the status of their near-vision acuity in the past 7 days, under mesopic conditions.

In the single item PGIC question, the participants answer a single-item question about their global impression of change in their near-vision acuity under mesopic conditions.

At the end of this study, in all three cohorts (or at least one cohort) of Part A, the ophthalmic solutions of the compound of Formula I administered bilaterally for 14 days demonstrate acceptable (or improved) safety and tolerability profiles in the participants. Additionally, in all three cohorts (or at least one cohort) of Part A, the ophthalmic solutions of the compound of Formula I administered bilaterally for 14 days result in systemic exposures in the participants with sufficient safety margins. Additionally, in all three cohorts (or at least one cohort) of Part A, the ophthalmic solutions of the compound of Formula I administered bilaterally for 14 days demonstrate a decrease in pupil diameter in the participants as compared with vehicle. Additionally, in all three cohorts (or at least one cohort) of Part A, the ophthalmic solutions of the compound of Formula I administered bilaterally for 14 days demonstrate a numeric improvement (e.g. two or three lines) in distance corrected near visual acuity in the participants as compared with vehicle. Additionally, in all three cohorts (or at least one cohort) of Part A, the ophthalmic solutions of the compound of Formula I administered bilaterally for 14 days demonstrate an improved depth of focus in the participants as compared with vehicle. Additionally, in all three cohorts (or at least one cohort) of Part A, the ophthalmic solutions of the compound of Formula I administered bilaterally for 14 days demonstrate more positive answers on the five patient reported outcome questionnaires/questions provided by the participants as compared with vehicle. Additionally, in all three cohorts (or at least one cohort) of Part A, the ophthalmic solutions of the compound of Formula I administered bilaterally for 14 days demonstrate whitening of the eye or decreasing any hyperemia or eye redness.

Also at the end of this study, in both cohorts (or at least one cohort) of Part B, the ophthalmic solutions of the compound of Formula I administered bilaterally for 14 days demonstrate acceptable safety and tolerability profiles in the participants. Additionally, in Cohort 7 of Part B, the ophthalmic solutions of the compound of Formula I demonstrate acceptable (or improved, i.e. a comparative reduction in the incidence of one or more of the adverse events associated with administration of pilocarpine) safety and tolerability profiles in the participants as compared with 1.25% (w/v) pilocarpine. Additionally, in Cohort 7 of Part B, the ophthalmic solutions of the compound of Formula I administered bilaterally for 14 days demonstrate similar (or better) improvement (e.g. two or three lines) in distance corrected near (and/or intermediate) visual acuity in the participants as compared with 1.25% (w/v) pilocarpine. Additionally, in Cohort 7 of Part B, the ophthalmic solutions of the compound of Formula I administered bilaterally for 14 days demonstrate similar numbers of positive answers on the five patient reported outcome questionnaires/questions provided by the participants as compared with 1.25% (w/v) pilocarpine. Additionally, in Cohort 7 of Part B, the ophthalmic solutions of the compound of Formula I administered bilaterally for 14 days demonstrate whitening of the eye or decreasing any hyperemia or eye redness. Additionally, in Cohort 8 of Part B, the ophthalmic solutions of the compound of Formula I administered bilaterally for 14 days demonstrate a numeric improvement (e.g. two or three lines) in distance corrected near (and/or intermediate) visual acuity in the participants as compared with vehicle. Additionally, in Cohort 8 of Part B, the ophthalmic solutions of the compound of Formula I administered bilaterally for 14 days demonstrate an improved depth of focus in the participants as compared with vehicle. Additionally, in Cohort 8 of Part B, the ophthalmic solutions of the compound of Formula I administered bilaterally for 14 days demonstrate more positive answers on the five patient reported outcome questionnaires/questions provided by the participants as compared with vehicle. Additionally, in Cohort 8 of Part B, the ophthalmic solutions of the compound of Formula I administered bilaterally for 14 days demonstrate whitening of the eye or decreasing any hyperemia or eye redness.

### Example 6

### Treatment of Red Eye

A 38-year-old woman complains about eye redness caused by seasonal allergies. A physician identifies the patient as having an OSH score of +2, and instructs her to administer to both of her eyes a composition comprising a compound of Formula I once or twice a day. Within the first day of administering the compound of Formula I, the patient reports decreased eye redness despite having on-going seasonal allergies, and her physician identifies the patient as having an OSH score of +0.5.

A 58-year old man without any underlying health problems notices his eyes are red, making him appear tired at work. He administers to both of his eyes a composition comprising a compound of Formula I in order to increase the whiteness of his eyes for cosmetic reasons. After administering the composition, his eyes are noticeably whiter, leading to his improved confidence in the workplace.

A 26-year old woman complains to her doctor that she suffers from red eyes due to minor eye irritations. The doctor prescribes a composition comprising a compound of Formula I and instructs her to administer the composition once or twice a day. The doctor also tells the woman that many other marketed compositions for reducing eye redness are associated with side effects such as rebound hyperemia, dependency, and/or tachyphylaxis and that the woman should report any such side effects. A few weeks later the woman returns to her doctor for a follow-up visit and indicates that not only are her eyes no longer as red as they used to be but that she has not observed any of the rebound hyperemia, dependency, or tachyphylaxis that has been associated with other medications for reducing eye redness.

A 45-year old man has been using a composition comprising brimonidine to whiten his eyes when he is out at clubs. He has generally been satisfied with the product because it takes effect in less than a minute. Upon recommendation from a friend, he asks his doctor about possibly switching to a composition comprising the compound of Formula I since his friend told him that it works faster than brimonidine. The doctor agrees to prescribe composition comprising the compound of Formula I. Upon a follow-up visit, the man reports that the new composition is indeed more effective in reducing eye redness in quite a bit less than a minute and faster than the brimonidine composition.

### Example 7

### In Vivo Rabbit Miosis Model

Female Dutch Belted rabbits (Covance, Princeton, New Jersey) weighing between 2-4 kg were used for these studies. All experimental animals received the selected form of the compound of Formula I (compound 1), compound 2, brimonidine (compound 4), or vehicle in in a single unilateral topical dose in the right eye. For topical dosing, eye drops (volume=35 μl) were instilled into the lower conjunctival sac of the test eye.

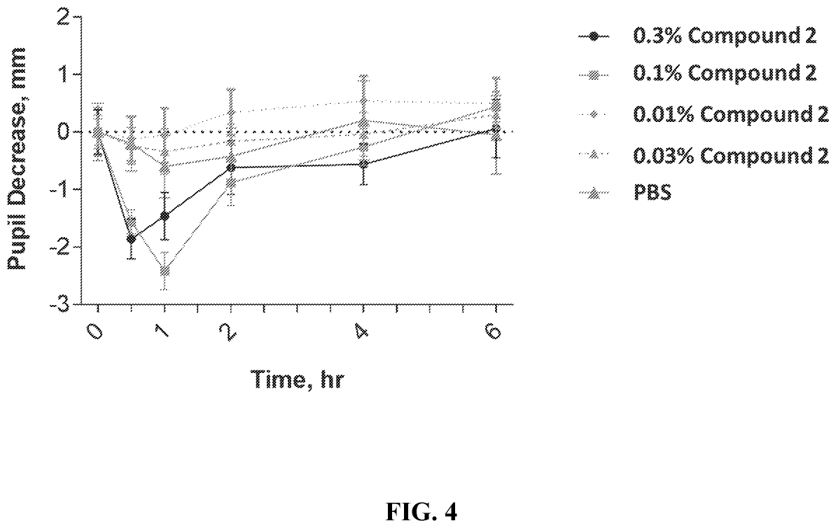

Pupil diameter was measured in both the treated and untreated eye with an Optistick to the nearest 0.5 mm. In all studies, baseline pupil diameter measurements were taken prior to drug administration and then at 0.5, 1, 2, 3 and 4 hours post dose. All studies were done under low-light conditions in which a red photography light is used providing 2-10 Lux of light. The results are shown in FIG. 4 to FIG. 7.

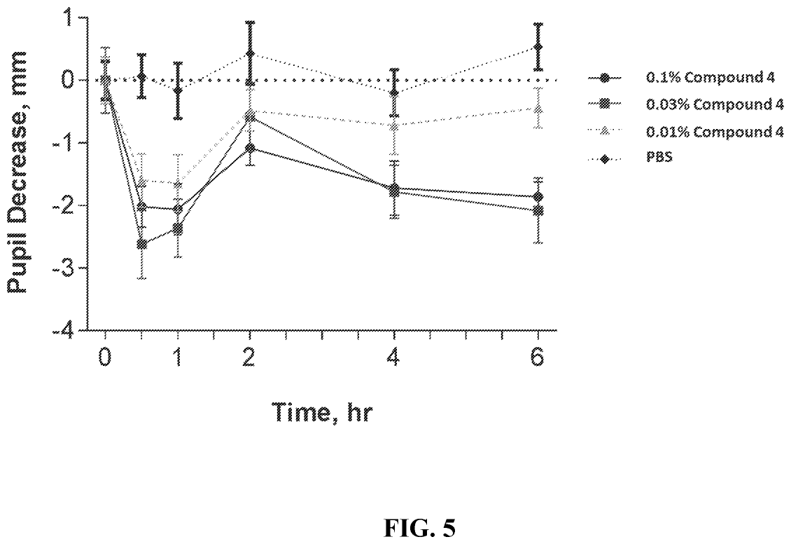

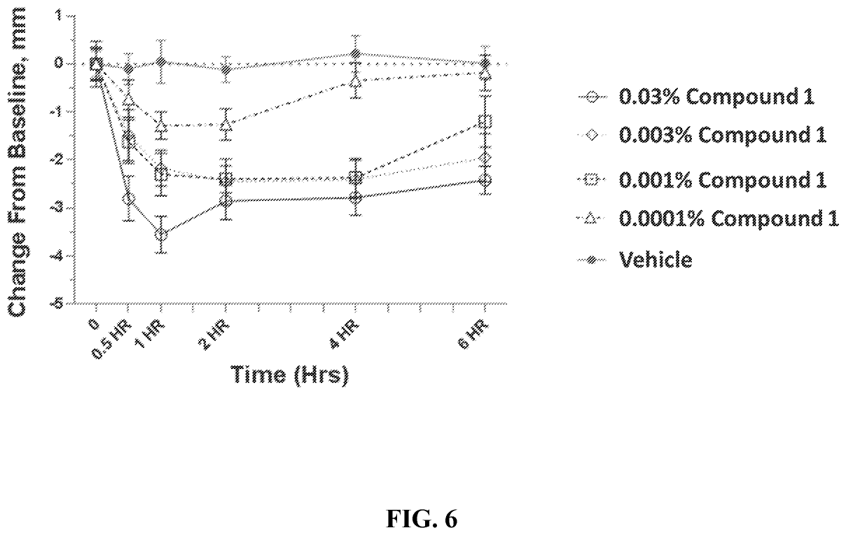

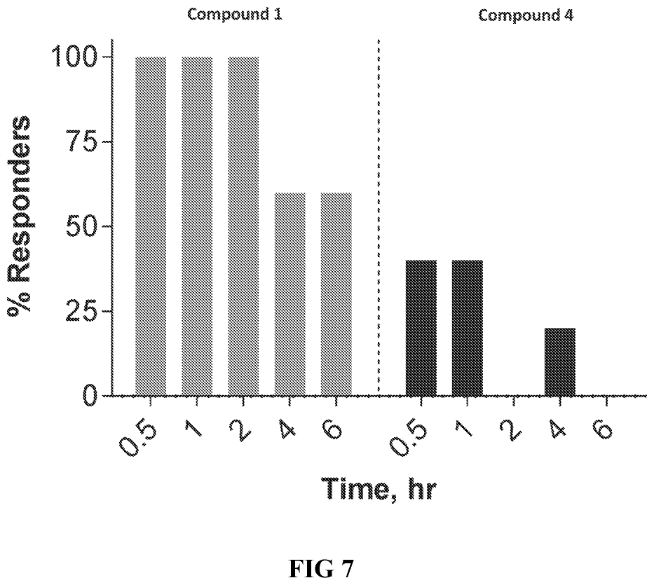

As mentioned in Example 1, it would have been expected that alpha-2-adrenergic receptor pan-agonists such as compounds 2 and 3 and the compound of Formula I (compound 1) would have similar miotic efficacy (peak and duration) in rabbit to that of brimonidine (compound 4). However, both the compounds of entries 2 and 3 have miotic efficacy that is much less than brimonidine, as can be seen from FIG. 4 to FIG. 7. In spite of having similar potency to brimonidine at the alpha-2A-adrenergic receptor, the compound of Formula I unexpectedly is about 30 to about 100 times more potent than brimonidine in the rabbit model (for example, the 0.001% solution of the compound in Formula I in FIG. 6 has a similar dose miotic response curve as seen with the 0.1% solution of brimonidine in FIG. 5 in terms of peak pupil decrease and duration of decrease, with the 0.001% composition of the compound of Formula I therefore having more miotic efficacy than the 0.1% dose tested for brimonidine). The compound of Formula I had the best miotic effect when compared with other alpha adrenergic receptor agonists including brimonidine. For example, as shown in FIG. 6, the compound of Formula I (compound 1) showed a very potent dose miotic response in DB rabbits-Scotopic condition (<10 lux). Furthermore, as shown in FIG. 7, responder analysis of the subjects (rabbits) with >2.5 mm pupil change showed that the compound of Formula I is more efficacious than brimonidine (compound 4) in Dutch Belted rabbits (n=6) under scotopic condition (<10 lux). In particular, FIG. 7 also shows that a greater amount of pupil size reduction can be achieved for a longer amount of time for the Compound of Formula I as compared to brimonidine since virtually all animals dosed with the compound of Formula I had a greater than 2.5 mm reduction in pupil size from baseline at two hours after dosing, and more than half had the same pupil size reduction after 6 hours, whereas much less than half of the animals dosed with brimonidine exhibited the same pupil size reduction even at a half hour after dosing, and virtually none of the animals exhibited the same pupil size reduction after 6 hours.

Additionally, as can be seen in FIG. 7 and in the comparison of FIGS. 5 and 6, the compound of Formula I shows a greater magnitude of pupil size reduction and therapeutic activity when compared to brimonidine.

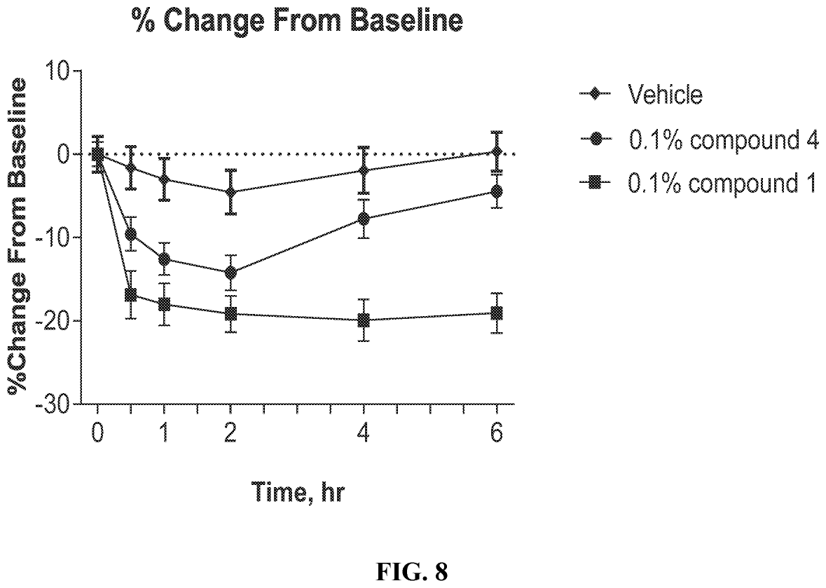

In addition, in a similar experiment performed under room lighting conditions, rabbits dosed with the compound of Formula I (Compound 1) still had greater miotic action than brimonidine (Compound 4) both in terms of peak pupil constriction and in terms of duration of action, as can be seen from FIG. 8.

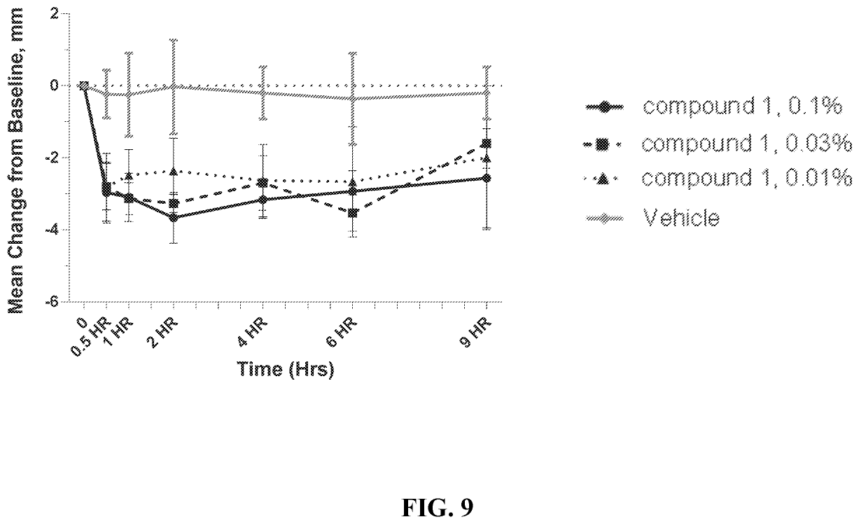

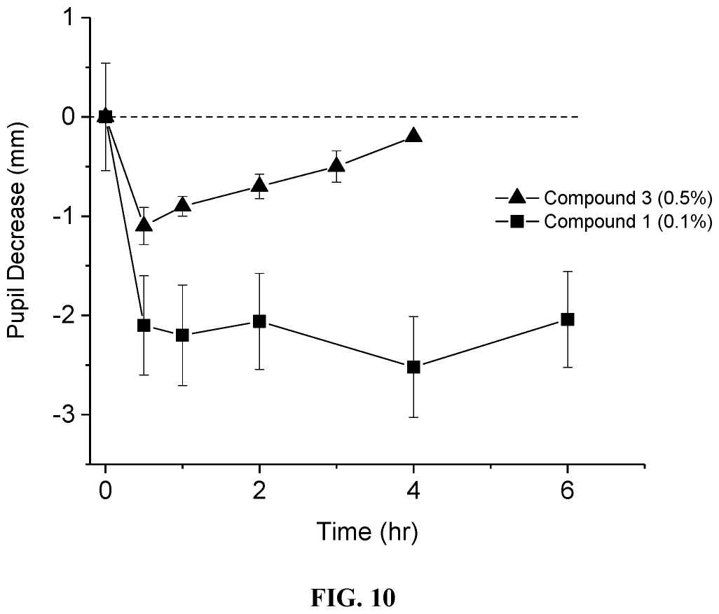

Additionally, as can be seen from FIG. 9, the compound of Formula I (Compound 1) continues to show significant pupil constriction even at 9 hours after dosing. In addition, as shown in FIG. 10, the compound of Formula I also showed a greater effect on pupil constriction when compared to Compound 3.

Such results would not have been expected since based on the in vitro data from Example 1, all the compounds would have been expected to have very similar miotic activity.

### Example 8

### Treatments of Presbyopia

A 56-year-old woman complains of an inability to focus on text when reading up close, which is interfering with her ability of read documents at work, as well as reading books and news articles. The problem seems worse under lower lighting conditions, such as the dim lighting in a restaurant. The visual degradation had been occurring over time, but in recent months, the woman's inability to focus on text when read close up had been more pronounced such as to interfere with her quality of life. The woman is seen by an ophthalmologist who performs a visual acuity test in which she is asked to read lines of letters on an eye chart without the assistance of glasses or contacts (neither of which she wears anyway). She finds that she is only able to read the first four lines on the chart, when a person with normal vision should be able to read six. Based on the woman's age and results of the test, she is diagnosed with presbyopia. The woman is reluctant to have to obtain reading glasses or wear contact lenses and asks if there are any other medical treatments. She is instructed to administer to her eyes one of the pharmaceutical compositions described herein one or two times a day. Starting with the first couple of doses, the patient reports improved vision when reading text up close. On a follow-up visit to the ophthalmologist, she is again asked to read lines of letters on an eye chart (she is still administering the composition with the compound of Formula I to her eyes), and this time she is able to read the first six lines, a two line improvement over her previous results prior to administering the pharmaceutical composition to her eyes.

A 48-year old man has noticed that his near vision has been deteriorating over the last few years such that he often has to hold reading material almost out to arm's length in order to be able to read the print, especially when the ambient light is dim. The man visits his ophthalmologist who performs a basic eye exam and refraction assessment. Based on the examination, the ophthalmologist prescribes a composition comprising brimonidine to constrict the man's pupil to treat the presbyopia, and the man is instructed to administer the composition to his eyes daily as needed. After a week the man returns to his ophthalmologist and indicates that while the brimonidine composition is working effectively to treat the presbyopia (he no longer has to hold reading documents out at almost arm's length), he finds that he must administer the composition three or more times a day. The ophthalmologist switches the man to one of the pharmaceutical compositions described herein and instructs him to administer the new composition to his eyes as needed as he had done with the brimonidine composition. The ophthalmologist follows up with the man about a week later and the man reports that the composition he was switched to works as well as the brimonidine composition, but unlike the brimonidine composition, the man only need to administer the composition to his eyes once (or sometimes twice) a day as opposed to three or more times a day.

A 66-year-old man reports dissatisfaction with his bifocal glasses, which, due to the two different refractive indices in the component parts of the lenses, have caused him to nearly fall several times when descending stairs. His ophthalmologist, having previously diagnosed him with presbyopia, instructs him to administer once daily to his eyes one of the pharmaceutical compositions described herein. After administration, the patient finds that his near and distance vision are improved, and that he no longer requires near and distance visual correction with glasses.

A 59-year-old woman who was previously diagnosed with presbyopia wishes to look into alternatives to the glasses and contact lenses that she has been wearing since her diagnosis. Her ophthalmologist prescribes as composition comprising brimonidine to her and instructs her to administer the composition to her eyes as needed. After a few days of administering the composition to her eyes, she calls the ophthalmologist and tells her that although the brimonidine composition is working to improve her vision such that she doesn't need her glasses or contacts to read text when it is up close, she finds that she generally needs to use greater amounts of the composition to achieve satisfactory results than the prescribing information indicates is usually needed and that she is experiencing some of the side effects associated with brimonidine such as sedation. The ophthalmologist notes that the woman has very dark irises and suspects that some amount of the brimonidine (which has fairly high melanin binding) is likely binding to the melanin in the woman's irises thus requiring her to administer more of the brimonidine composition to provide enough free (not bound to melanin) brimonidine to achieve satisfactory effects. The ophthalmologist changes the woman's prescription to be for one of the pharmaceutical compositions described herein and instructs her to administer the composition to her eyes as she did with the brimonidine composition. About a week later the ophthalmologist follows up with the woman who indicates that she can now obtain a satisfactory improvement in her near-reading vision with less drops of the composition comprising a compound of Formula I than she did with the brimonidine composition and is not experiencing the side effects associated with alpha-2-adrenergic receptor agonists such as sedation.

### Example 9

### Treatments of Visual Glare, Starbursts, and Halos

A 45-year-old man decides to undergo LASIK surgery. The surgeon who will perform the surgery evaluates the patient and determines that he is a suitable candidate for the surgery, but is told that side effects of the surgery include visual glare, visual starbursts, and visual halos, especially at night. The surgeon performs the surgery without any noticeable issues and the patient is discharged. A day later the patient drives home during the evening for the first time since the surgery and notices what appear to be starbursts of light emanating from the headlights and rear lights of other cars, as well as from street lights, and also glare coming from those light sources that interfere with is vision. The patient also observes diffuse rings around some of the street lights and illuminated street signs. Upon consultation, the surgeon confirms that such visual disturbances are indeed the visual glare, visual starburst, and visual halo side effects often seen after LASIK surgery, and the patient is prescribed one of the pharmaceutical compositions described herein, which the patient administers to his eyes in accordance with the package instructions. The next time the patient drives home during the evening, the glare, starbursts, and halos are significantly reduced such that he is no longer bothered by them.

A 61-year-old woman decides that she wishes to undergo LASIK surgery so as to no longer need to wear glasses. After being evaluated by a surgeon she is found to be a viable candidate for the procedure. The woman undergoes the procedure and is prescribed a composition comprising brimonidine and instructed to administer the composition to her eyes as needed should she develop any visual glare, visual starbursts, and visual halos, which are common side effects of LASIK. The first time she drives at night after the procedure she indeed notices visual glare, as well as starbursts and halos around light sources and some illuminated signs. As advised by her surgeon, she begins administering the brimonidine composition before here early morning and nighttime commutes. However, she finds that she generally needs to administer more than the standard dosing in order to have a satisfactory effect in reducing the visual disturbances, and the increased amount of brimonidine is having some side effects such as sedation. The woman visits her surgeon and tells her of the situation. The surgeon indicates that alpha-2-adrenergic receptor agonists such as brimonidine can sometimes be associated with side effects such as sedation. The surgeon believes that the problem may be that the woman's dark irises might be binding the brimonidine, since brimonidine is known to bind to melanin, and that the woman might be requiring the increased doses of the brimonidine due to this binding effect. The surgeon then prescribes one of the pharmaceutical compositions described herein to the woman and instructs her to administer the new composition to her eyes as needed as she had done with the brimonidine composition. The woman is then instructed to follow up with the surgeon after her next night drive. The woman does as instructed and reports back to the surgeon indicating that she needed much less drops of the composition than she did with the brimonidine composition in order to makes the visual glare, visual starbursts, and visual halos subside.

A 59-year-old man has been found as a viable candidate for LASIK surgery and chooses to undergo the procedure rather than have to continuing wearing glasses or contacts. The man is a long-distance truck driver and he works a long schedule in which he drives for 9 to 13 hours (sometimes more) at night and early morning and sleeps during the day. Because the man's driving route is located in a norther region of the United States, almost all of the man's 9 to 13 (or more) hours of night driving are in darkness, especially in the winter. The man undergoes the surgery, but is told that some LASIK patients can have visual disturbances such as visual glare, visual starbursts, and visual halos, especially at night. Given that the man's work schedule, he is given a prescription for a composition comprising brimonidine and told to administer the composition to his eyes as needed should he experience the nighttime visual disturbances. Shortly after the surgery, when the man returns to his night driving duties, he notices visual glare, visual starbursts, and visual halos coming from light sources such as headlights and taillights and illuminated highway signs. The man does as his surgeon ordered and begins administering the brimonidine composition to his eyes. He finds that even though the brimonidine composition reduces the visual glare, visual starbursts, and visual halos, he has to administer the composition 3 or 4 times during his long drive. He contacts his ophthalmologist and asks if there are any other medicines he can use to deal with the visual disturbances which might be longer acting. The ophthalmologist prescribes one of the pharmaceutical compositions described herein and the man is told to use that composition instead. The man happily finds out that he only needs to administer the composition only once (or sometimes twice) during his long drive.

### Example 10

### Night Vision Improvements

A 62-year-old woman has noticed that she is having issues seeing the street names on street signs with good contrast when she is driving at night. The woman consults with her ophthalmologist who, upon hearing the patient's description and performing a visual acuity test under low light conditions, prescribes one of the pharmaceutical compositions described herein, which the patient administers to her eyes in accordance with the package instructions. The next time the patient is driving at night, the patient finds that she can see with much better contrast and is thus better able to read street signs.

A 45-year-old man works as a nighttime security guard and complains that he is having issues seeing objects with good contrast at night. Because this interferes with his job, he meets with an ophthalmologist who prescribes a composition comprising brimonidine and instructs the man to administer the composition to his eyes. However, he finds that he often needs to administer fairly large amounts of the composition to his eyes in order to obtain a satisfactory effect, and these larger amounts are beginning to cause side effects that are sometimes seem with brimonidine (in particular sedation). He consults with his ophthalmologist who believes that the problem may be that the brimonidine is being bound by the melanin in the man's irises, which are quite dark. The ophthalmologist switches the man to one of the pharmaceutical compositions described herein and instructs him to administer that composition instead of the brimonidine composition and report the results back to the ophthalmologist. The man does so, and when he reports back to the ophthalmologist a few days later, he indicates that he can obtain satisfactory night vision improvement with much less of the composition and consequently does not suffer from the side effects that he was previously experiencing.

### Example 11

### Treatments of Night Myopia

A 56-year-old woman notices that, although she has no significant problems with distance vision during the day, during the night she seems to have a difficult time focusing of distant objects (e.g. street signs). She goes to see an ophthalmologist who performs some visual acuity testes under both normal lighting conditions and under low light conditions. The ophthalmologist confirms that the woman does not have any significant issues with distance vision under the normal lighting conditions, but that she does suffer from myopia under the low light conditions. She is prescribed one of the pharmaceutical compositions described herein, which she administers to her eyes in accordance with the package instructions. The patient the finds that her ability to focus on distant objects at night is now as good as her ability to do so during the day.

A 61-year-old man who works a full 9 to 10-hour work schedule almost exclusively at night has noticed that he has problems focusing on distant objects at night while his colleagues of similar age do not have that problem. He also notices that he does not, in turn, have the same problem focusing on distant objects during the day. He visits an ophthalmologist who diagnoses the man with night myopia and prescribes a composition comprising brimonidine and instructs him to administer it to his eyes as needed. The man finds that administering the brimonidine composition to his eyes gives him a notable improvement is his ability to focus on distant objects at night, but it has a fairly short duration of action such that he often needs to administer the composition to his eyes three or four times during his waking hours so as to maintain a satisfactory effect. He calls his ophthalmologist who then prescribes one of the pharmaceutical compositions described herein and instructs him to use that composition instead. The man does so and finds that he now only needs to administer the composition only once or twice during his waking hours.

### Example 12

### Treatments of Presbyopia with Eye Redness Reduction/Increased Whitening

A 56-year-old woman complains of an inability to focus on text when reading up close, which is interfering with her ability of read documents at work, as well as reading books and news articles. The problem seems worse under lower lighting conditions, such as the dim lighting in a restaurant. The visual degradation had been occurring over time, but in recent months, the woman's inability to focus on text when read close up had been more pronounced such as to interfere with her quality of life. The woman is seen by an ophthalmologist who performs a visual acuity test in which she is asked to read lines of letters on an eye chart without the assistance of glasses or contacts (neither of which she wears anyway). She finds that she is only able to read the first four lines on the chart, when a person with normal vision should be able to read six. Based on the woman's age and results of the test, she is diagnosed with presbyopia. The woman is reluctant to have to obtain reading glasses or wear contact lenses and asks if there are any other medical treatments. She is instructed to administer to her eyes one of the pharmaceutical compositions described herein one or two times a day. Starting with the first couple of doses, the patient reports improved vision when reading text up close. On a follow-up visit to the ophthalmologist, she is again asked to read lines of letters on an eye chart (she is still administering the composition with the compound of Formula I to her eyes), and this time she is able to read the first six lines, a two line improvement over her previous results prior to administering the pharmaceutical composition to her eyes. She also notices that the redness that is often present in her eyes during allergy season is also reduced by the composition.

A 48-year old man has noticed that his near vision has been deteriorating over the last few years such that he often has to hold reading material almost out to arm's length in order to be able to read the print, especially when the ambient light is dim. The man visits his ophthalmologist who performs a basic eye exam and refraction assessment. Based on the examination, the ophthalmologist prescribes a composition comprising brimonidine to constrict the man's pupil to treat the presbyopia, and the man is instructed to administer the composition to his eyes daily as needed. After a week the man returns to his ophthalmologist and indicates that while the brimonidine composition is working effectively to treat the presbyopia (he no longer has to hold reading documents out at almost arm's length), he finds that he must administer the composition three or more times a day. The ophthalmologist switches the man to one of the pharmaceutical compositions described herein and instructs him to administer the new composition to his eyes as needed as he had done with the brimonidine composition. The ophthalmologist follows up with the man about a week later and the man reports that the composition he was switched to works as well as the brimonidine composition, but unlike the brimonidine composition, the man only need to administer the composition to his eyes once (or sometimes twice) a day as opposed to three or more times a day. In addition, the man also reports that his eyes appear whiter with administration of the composition, which has given him a confidence boost.

A 66-year-old man reports dissatisfaction with his bifocal glasses, which, due to the two different refractive indices in the component parts of the lenses, have caused him to nearly fall several times when descending stairs. His ophthalmologist, having previously diagnosed him with presbyopia, instructs him to administer once daily to his eyes one of the pharmaceutical compositions described herein. After administration, the patient finds that his near and distance vision are improved, and that he no longer requires near and distance visual correction with glasses. He also finds that the redness in his eyes that is induced by late nights reading books has been reduced by the composition.

A 59-year-old woman who was previously diagnosed with presbyopia wishes to look into alternatives to the glasses and contact lenses that she has been wearing since her diagnosis. Her ophthalmologist prescribes as composition comprising brimonidine to her and instructs her to administer the composition to her eyes as needed. After a few days of administering the composition to her eyes, she calls the ophthalmologist and tells her that although the brimonidine composition is working to improve her vision such that she doesn't need her glasses or contacts to read text when it is up close, she finds that she generally needs to use greater amounts of the composition to achieve satisfactory results than the prescribing information indicates is usually needed and that she is experiencing some of the side effects associated with brimonidine such as sedation. The ophthalmologist notes that the woman has very dark irises and suspects that some amount of the brimonidine (which has fairly high melanin binding) is likely binding to the melanin in the woman's irises thus requiring her to administer more of the brimonidine composition to provide enough free (not bound to melanin) brimonidine to achieve satisfactory effects. The ophthalmologist changes the woman's prescription to be for one of the pharmaceutical compositions described herein and instructs her to administer the composition to her eyes as she did with the brimonidine composition. About a week later the ophthalmologist follows up with the woman who indicates that she can now obtain a satisfactory improvement in her near-reading vision with less drops of the composition comprising a compound of Formula I than she did with the brimonidine composition and is not experiencing the side effects associated with alpha-2-adrenergic receptor agonists such as sedation. Moreover, she also reports that the eye redness that she normally observed when she would wake up after not getting much sleep has been significantly reduced by the composition.

### Example 13

### Treatments of Visual Glare, Starbursts, and Halos with Eye Redness Reduction/Increased Whitening

A 45-year-old man decides to undergo LASIK surgery. The surgeon who will perform the surgery evaluates the patient and determines that he is a suitable candidate for the surgery, but is told that side effects of the surgery include visual glare, visual starbursts, and visual halos, especially at night. The surgeon performs the surgery without any noticeable issues beyond some eye redness and the patient is discharged. A day later the patient drives home during the evening for the first time since the surgery and notices what appear to be starbursts of light emanating from the headlights and rear lights of other cars, as well as from street lights, and also glare coming from those light sources that interfere with is vision. The patient also observes diffuse rings around some of the street lights and illuminated street signs. Upon consultation, the surgeon confirms that such visual disturbances are indeed the visual glare, visual starburst, and visual halo side effects often seen after LASIK surgery, and the patient is prescribed one of the pharmaceutical compositions described herein, which the patient administers to his eyes in accordance with the package instructions. The next time the patient drives home during the evening, the glare, starbursts, and halos are significantly reduced such that he is no longer bothered by them. Moreover, the surgeon and patient notice that the eye redness has been reduced by the composition.

A 61-year-old woman decides that she wishes to undergo LASIK surgery so as to no longer need to wear glasses. After being evaluated by a surgeon she is found to be a viable candidate for the procedure. The woman undergoes the procedure and is prescribed a composition comprising brimonidine and instructed to administer the composition to her eyes as needed should she develop any visual glare, visual starbursts, and visual halos, which are common side effects of LASIK. The first time she drives at night after the procedure she indeed notices visual glare, as well as starbursts and halos around light sources and some illuminated signs. As advised by her surgeon, she begins administering the brimonidine composition before here early morning and nighttime commutes. However, she finds that she generally needs to administer more than the standard dosing in order to have a satisfactory effect in reducing the visual disturbances, and the increased amount of brimonidine is having some side effects such as sedation. The woman visits her surgeon and tells her of the situation. The surgeon indicates that alpha-2-adrenergic receptor agonists such as brimonidine can sometimes be associated with side effects such as sedation. The surgeon believes that the problem may be that the woman's dark irises might be binding the brimonidine, since brimonidine is known to bind to melanin, and that the woman might be requiring the increased doses of the brimonidine due to this binding effect. The surgeon then prescribes one of the pharmaceutical compositions described herein to the woman and instructs her to administer the new composition to her eyes as needed as she had done with the brimonidine composition. The woman is then instructed to follow up with the surgeon after her next night drive. The woman does as instructed and reports back to the surgeon indicating that she needed much less drops of the composition than she did with the brimonidine composition in order to makes the visual glare, visual starbursts, and visual halos subside. The woman also reports that she has noticed that after starting administration of the composition her eyes appear whither than they used to be.

A 59-year-old man has been found as a viable candidate for LASIK surgery and chooses to undergo the procedure rather than have to continuing wearing glasses or contacts. The man is a long-distance truck driver and he works a long schedule in which he drives for 9 to 13 hours (sometimes more) at night and early morning and sleeps during the day. Because the man's driving route is located in a norther region of the United States, almost all of the man's 9 to 13 (or more) hours of night driving are in darkness, especially in the winter. The man undergoes the surgery, but is told that some LASIK patients can have visual disturbances such as visual glare, visual starbursts, and visual halos, especially at night. Given that the man's work schedule, he is given a prescription for a composition comprising brimonidine and told to administer the composition to his eyes as needed should he experience the nighttime visual disturbances. Shortly after the surgery, when the man returns to his night driving duties, he notices visual glare, visual starbursts, and visual halos coming from light sources such as headlights and taillights and illuminated highway signs. The man does as his surgeon ordered and begins administering the brimonidine composition to his eyes. He finds that even though the brimonidine composition reduces the visual glare, visual starbursts, and visual halos, he has to administer the composition 3 or 4 times during his long drive. He contacts his ophthalmologist and asks if there are any other medicines he can use to deal with the visual disturbances which might be longer acting. The ophthalmologist prescribes one of the pharmaceutical compositions described herein and the man is told to use that composition instead. The man happily finds out that he only needs to administer the composition only once (or sometimes twice) during his long drive. In addition, the man also notices that the eye redness he suffered from due to the low humidity of the climate in which he drives his truck has been reduced by using the composition.

### Example 14

### Night Vision Improvements with Eye Redness Reduction/Increased Whitening

A 62-year-old woman has noticed that she is having issues seeing the street names on street signs with good contrast when she is driving at night. The woman consults with her ophthalmologist who, upon hearing the patient's description and performing a visual acuity test under low light conditions, prescribes one of the pharmaceutical compositions described herein, which the patient administers to her eyes in accordance with the package instructions. The next time the patient is driving at night, the patient finds that she can see with much better contrast and is thus better able to read street signs. She also notices that the redness in her eyes associated with seasonal allergies has also been reduced by the composition.

A 45-year-old man works as a nighttime security guard and complains that he is having issues seeing objects with good contrast at night. Because this interferes with his job, he meets with an ophthalmologist who prescribes a composition comprising brimonidine and instructs the man to administer the composition to his eyes. However, he finds that he often needs to administer fairly large amounts of the composition to his eyes in order to obtain a satisfactory effect, and these larger amounts are beginning to cause side effects that are sometimes seem with brimonidine (in particular sedation). He consults with his ophthalmologist who believes that the problem may be that the brimonidine is being bound by the melanin in the man's irises, which are quite dark. The ophthalmologist switches the man to one of the pharmaceutical compositions described herein and instructs him to administer that composition instead of the brimonidine composition and report the results back to the ophthalmologist. The man does so, and when he reports back to the ophthalmologist a few days later, he indicates that he can obtain satisfactory night vision improvement with much less of the composition and consequently does not suffer from the side effects that he was previously experiencing. He also reports that his eyes have gotten whiter than they were before starting use of the composition.

### Example 15

### Treatments of Night Myopia with Eye Redness Reduction/Increased Whitening

A 56-year-old woman notices that, although she has no significant problems with distance vision during the day, during the night she seems to have a difficult time focusing of distant objects (e.g. street signs). She goes to see an ophthalmologist who performs some visual acuity testes under both normal lighting conditions and under low light conditions. The ophthalmologist confirms that the woman does not have any significant issues with distance vision under the normal lighting conditions, but that she does suffer from myopia under the low light conditions. She is prescribed one of the pharmaceutical compositions described herein, which she administers to her eyes in accordance with the package instructions. The patient the finds that her ability to focus on distant objects at night is now as good as her ability to do so during the day. In addition, she also notices that here eyes appear whiter now that she has started using the composition.

A 61-year-old man who works a full 9 to 10-hour work schedule almost exclusively at night has noticed that he has problems focusing on distant objects at night while his colleagues of similar age do not have that problem. He also notices that he does not, in turn, have the same problem focusing on distant objects during the day. He visits an ophthalmologist who diagnoses the man with night myopia and prescribes a composition comprising brimonidine and instructs him to administer it to his eyes as needed. The man finds that administering the brimonidine composition to his eyes gives him a notable improvement is his ability to focus on distant objects at night, but it has a fairly short duration of action such that he often needs to administer the composition to his eyes three or four times during his waking hours so as to maintain a satisfactory effect. He calls his ophthalmologist who then prescribes one of the pharmaceutical compositions described herein and instructs him to use that composition instead. The man does so and finds that he now only needs to administer the composition only once or twice during his waking hours. In addition, the man also notices that the redness in his eyes that is often caused by pollen release in the spring has been reduced after starting to use the composition.

Throughout this specification reference is made to publications such as US and foreign patent applications, journal articles, book chapters, and others. All such publications are expressly incorporated by reference in their entirety, including supplemental/supporting information sections published with the corresponding references, for all purposes unless otherwise indicated. To the extent that any recitations in the incorporated references conflict with any recitations herein, the recitations herein will control.

The foregoing descriptions details methods that can be employed to treat various ocular conditions, and represents the best mode contemplated. It should not be construed as limiting the overall scope hereof; rather, the ambit of the present disclosure is to be governed only by the lawful construction of the appended claims.

What is claimed is:

1. A method for reducing redness of an eye or increasing whiteness of an eye in an individual in need thereof, the method comprising administering to the individual a therapeutically effective amount of a compound of Formula I: 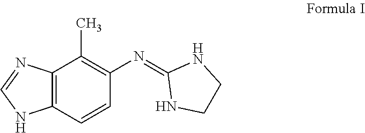 [SMILES: Cc1c(N=C2NCCN2)ccc2[nH]cnc12]
or a pharmaceutically acceptable salt thereof.

2. The method of claim 1, wherein the method is for reducing redness of the eye.

3. The method of claim 2, wherein the method is for reducing redness of the eye caused by minor eye irritations.

4. The method of claim 2, wherein the method is for reducing redness of the eye due to dilation of vessels of the conjunctiva, the episclera, the sclera, or a combination of two or more thereof.

5. The method of claim 2, wherein the method is for reducing chronic redness of the eye.

6. The method of claim 1, wherein the method is for increasing whiteness of the eye.

7. The method of claim 6, wherein the individual does not have an eye pathology.

8. The method of claim 1, wherein the method is for reducing redness of the eye caused by laser-assisted in situ keratomileusis eye surgery.

9. The method of claim 1, wherein the method reduces redness of the eye or increases whiteness of the eye within 5 seconds of administration.

10. The method of claim 1, wherein the method reduces redness of the eye or increases whiteness of the eye within 15 seconds of administration.

11. The method of claim 1, wherein the method reduces redness of the eye or increases whiteness of the eye within 30 seconds of administration.

12. The method of claim 1, wherein the method reduces redness of the eye or increases whiteness of the eye within 1 minute of administration.

13. The method of claim 1, wherein the method reduces redness of the eye or increases whiteness of the eye for at least 8 hours after administration.

14. The method of claim 1, wherein the method reduces redness of the eye or increases whiteness of the eye for at least 9 hours after administration.

15. The method of claim 1, wherein the method reduces redness of the eye or increases whiteness of the eye for at least 10 hours after administration.

16. The method of claim 1, wherein the method reduces redness of the eye or increases whiteness of the eye for at least 12 hours after administration.

17. The method of claim 1, wherein the method reduces redness of the eye or increases whiteness of the eye for at least 24 hours after administration.

18. The method of claim 1, wherein the compound of Formula I or the pharmaceutically acceptable salt thereof is administered to one eye of the individual or to both eyes of the individual.

19. The method of claim 1, comprising topically administering to the eye of the individual the therapeutically effective amount of the compound of Formula I or the pharmaceutically acceptable salt thereof.

20. The method of claim 1, wherein the compound of Formula I or the pharmaceutically acceptable salt thereof is administered to the individual as a pharmaceutical composition comprising the therapeutically effective amount of the compound of Formula I or the pharmaceutically acceptable salt thereof and a pharmaceutically acceptable excipient.

21. The method of claim 20, wherein the pharmaceutical composition comprises the compound of Formula I or the pharmaceutically acceptable salt thereof in an amount of about 0.001% (w/v) to about 0.02% (w/v).

22. The method of claim 20, wherein the pharmaceutical composition comprises the compound of Formula I or the pharmaceutically acceptable salt thereof in an amount of about 0.005% (w/v) to about 0.5% (w/v).

23. The method of claim 20, wherein the pharmaceutical composition comprises the compound of Formula I or the pharmaceutically acceptable salt thereof in an amount of about 0.01% (w/v).

24. The method of claim 20, wherein the pharmaceutical composition comprises the compound of Formula I or the pharmaceutically acceptable salt thereof in an amount of about 0.02% (w/v).

25. The method of claim 20, wherein the pharmaceutical composition comprises the compound of Formula I or the pharmaceutically acceptable salt thereof in an amount of about 0.03% (w/v).

26. The method of claim 20, wherein the pharmaceutical composition comprises the compound of Formula I or the pharmaceutically acceptable salt thereof in an amount of 0.1% (w/v).

27. The method of claim 20, wherein the pharmaceutical composition comprises the compound of Formula I or the pharmaceutically acceptable salt thereof in an amount of 0.3% (w/v).

28. The method of claim 20, wherein the pharmaceutical composition is a solution.

29. The method of claim 20, wherein the pharmaceutical composition is a dispersion.

30. The method of claim 20, comprising topically administering to the eye of the individual one drop of the pharmaceutical composition.

31. The method of claim 20, comprising topically administering to the eye of the individual two drops of the pharmaceutical composition.

32. The method of claim 20, wherein the pharmaceutical composition is an ocular implant, intracameral implant, intravitreal implant, sub-conjunctival implant, sub-Tenon's implant, punctum plug, canicular eluting implant, or ocular ring.

33. The method of claim 20, wherein the pharmaceutical composition comprises microspheres which comprise the compound of Formula (I) or the pharmaceutically acceptable salt thereof.

34. The method of claim 20, wherein the therapeutically effective amount of the compound of Formula (I) or the pharmaceutically acceptable salt thereof is an amount that is less than a therapeutically amount of brimonidine that is needed to reduce redness of the eye or to increase whiteness of the eye.
